SEQUENCE LISTING (41 sequences)

SEQ ID NO: 1 - protein, 10 aa, synthetic construct
GYEVHHQKLV

SEQ ID NO: 2 - protein, 28 aa, synthetic construct
CGKCGYEVHHQKLVPNVLKQHHVEYGCK

SEQ ID NO: 3 - protein, 28 aa, synthetic construct
CGKCGYEVHHQKLVPNGYEVHHQKLVCK

SEQ ID NO: 4 - protein, 25 aa, synthetic construct
KSSKSKKKFISEAIIHHLHSRHPGK

SEQ ID NO: 5 - protein, 12 aa, synthetic construct
QIYEVHHRGYTD

SEQ ID NO: 6 - protein, 12 aa, synthetic construct
NLHQYPTAMQAR

SEQ ID NO: 7 - protein, 12 aa, synthetic construct
SPPTYEIHHFSP

SEQ ID NO: 8 - protein, 12 aa, synthetic construct
YQIHHNTQKRGG

SEQ ID NO: 9 - protein, 12 aa, synthetic construct
DSVVPSYEVHHK

SEQ ID NO: 10 - protein, 12 aa, synthetic construct
TPNNTTSHRYPD

SEQ ID NO: 11 - protein, 12 aa, synthetic construct
GWYEIHHPSQRA

SEQ ID NO: 12 - protein, 12 aa, synthetic construct
SYELHHKPLHAQ

SEQ ID NO: 13 - protein, 12 aa, synthetic construct
GPHTYPPQIALT

SEQ ID NO: 14 - protein, 12 aa, synthetic construct
EMAPVASANLPP

SEQ ID NO: 15 - protein, 12 aa, synthetic construct
NMYEWNHKDPNM

SEQ ID NO: 16 - protein, 12 aa, synthetic construct
SPSHMYPSSLIQ

SEQ ID NO: 17 - protein, 12 aa, synthetic construct
QVYEVHHRGYTD

SEQ ID NO: 18 - DNA, 355 bp, synthetic construct
  source 1..355: mol_type = other DNA
gaggtgcagctgcaggagtctggggctgaactggtgaggcctggggtttcagtgaagctgtcctgcaagacttctgactacacattcaccagttattggttgcactggtttaagcagaggcctgaacaaggccttgagaggattggagagattgatcctagcaatggtgatgctgactacagtgagaagttcaagagcaaggccacactgactgtagacaaatcttccaccacagcctacatgcagctcgccagtctgacatctgaggactctgcggtctattactgtgcaagagaagcctacggccactactttgactactggggccaaggcaccactctcacagtctcctcag

SEQ ID NO: 19 - protein, 118 aa, synthetic construct
EVQLQESGAELVRPGVSVKLSCKTSDYTFTSYWLHWFKQRPEQGLERIGEIDPSNGDADYSEKFKSKATLTVDKSSTTAYMQLASLTSEDSAVYYCAREAYGHYFDYWGQGTTLTVSS

SEQ ID NO: 20 - protein, 8 aa, synthetic construct
DYTFTSYW

SEQ ID NO: 21 - protein, 8 aa, synthetic construct
IDPSNGDA

SEQ ID NO: 22 - protein, 11 aa, synthetic construct
AREAYGHYFDY

SEQ ID NO: 23 - DNA, 337 bp, synthetic construct
  source 1..337: mol_type = other DNA
gatgttgtgatgacccaaactccactctccctgcctgtcagtcttggagatcaagcctccatctcctgcagatccagtcagagccttgaacacagtaatggaaacacctatttacattggtacctgcagaagccaggccagtctccaaagctcctgatctacaaactttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaac

SEQ ID NO: 24 - protein, 112 aa, synthetic construct
DVVMTQTPLSLPVSLGDQASISCRSSQSLEHSNGNTYLHWYLQKPGQSPKLLIYKLSNRFSGVPDRFSGSGSGTDFTLKISRVEAEDLGVYFCSQSTHVPYTFGGGTKLEIK

SEQ ID NO: 25 - protein, 11 aa, synthetic construct
QSLEHSNGNTY

SEQ ID NO: 26 - skipped

SEQ ID NO: 27 - protein, 9 aa, synthetic construct
SQSTHVPYT

SEQ ID NO: 28 - DNA, 355 bp, synthetic construct
  source 1..355: mol_type = other DNA
caggtccaactgcaacagcctggggctgaactggtgaggcctggggtttcagtgaagctgtcctgcaagacttctgactacacattcaccagttattggttgcactggtttaagcagaggcctgaacaaggccttgagaggattggagagattgatcctagcaatggtgatgctgactacagtgagaagttcaagagcaaggccacactgactgtagacaaatcttccaccacagcctacatgcagctcgccagtctgacatctgaggactctgcggtctattactgtgcaagagaagcctacggccactactttgactactggggccaaggcaccactctcacagtctcctcag

SEQ ID NO: 29 - protein, 118 aa, synthetic construct
QVQLQQPGAELVRPGVSVKLSCKTSDYTFTSYWLHWFKQRPEQGLERIGEIDPSNGDADYSEKFKSKATLTVDKSSTTAYMQLASLTSEDSAVYYCAREAYGHYFDYWGQGTTLTVSS

SEQ ID NO: 30 - protein, 8 aa, synthetic construct
DYTFTSYW

SEQ ID NO: 31 - protein, 8 aa, synthetic construct
IDPSNGDA

SEQ ID NO: 32 - protein, 11 aa, synthetic construct
AREAYGHYFDY

SEQ ID NO: 33 - DNA, 337 bp, synthetic construct; identical to SEQ ID NO: 23
  source 1..337: mol_type = other DNA

SEQ ID NO: 34 - protein, 112 aa, synthetic construct; identical to SEQ ID NO: 24

SEQ ID NO: 35 - protein, 11 aa, synthetic construct
QSLEHSNGNTY

SEQ ID NO: 36 - skipped

SEQ ID NO: 37 - protein, 9 aa, synthetic construct
SQSTHVPYT

SEQ ID NO: 38 - protein, 8 aa, synthetic construct
DYKDDDDK

SEQ ID NO: 39 - protein, 42 aa, synthetic construct [sequence omitted]

SEQ ID NO: 40 - protein, 40 aa, synthetic construct
DAEFRHDSGYEVHHQKLVFFAEDVGSNKGAIIGLMVGGVV

SEQ ID NO: 41 - protein, 5 aa, synthetic construct
YEXHH

(19) United States
(12) Patent Application Publication
(10) Pub. No.: US 2024/0358805 A1
(43) Pub. Date: Oct. 31, 2024

(54) METHODS AND MATERIALS TO TREAT NEURODEGENERATIVE DISEASE

(71) Applicants: The Brigham and Women's Hospital, Inc., Boston, MA (US); Abyssinia Biologics, LLC, Durham, NH (US)

(72) Inventors: Dennis J. Selkoe, Jamaica Plain, MA (US); Lei Liu, Boston, MA (US); Robert Leslie Lawton, Gorham, MA (US)

(21) Appl. No.: 18/688,269

(22) PCT Filed: Aug. 30, 2022
(86) PCT No.: PCT/US2022/042073

§ 371 Date: Feb. 29, 2024

Related U.S. Application Data
(60) Provisional application No. 63/393,885, filed on Jul. 30, 2022.
(60) Provisional application No. 63/346,240, filed on May 26, 2022.
(60) Provisional application No. 63/238,329, filed on Aug. 30, 2021.
(60) Provisional application No. 63/238,252, filed on Aug. 30, 2021.

(51) Int. Cl. A61K 39/00; A61P 25/28; C07K 16/18; G01N 33/68

(52) U.S. Cl. CPC A61K 39/0007; A61P 25/28; C07K 16/18; G01N 33/6896; A61K 2039/6081; G01N 2800/2821

(57) ABSTRACT

Described herein are methods for making and using antibodies that bind specifically to soluble oligomers of amyloid beta protein (oAβ), and methods of use thereof in diagnosis and treatment of diseases related to formation of pathogenic oligomers of oAβ. Also provided are T helper cell (Th) carrier peptides that can be conjugated to peptide immunogens to stimulate targeted humoral immune responses to the desired antigen.

### CLAIM OF PRIORITY

This application claims the benefit of U.S. Provisional Application Ser. No. 63/238,252, filed on Aug. 30, 2021; 63/238,329, filed on Aug. 30, 2021; 63/346,240, filed on May 26, 2022; and 63/393,885, filed on Jul. 30, 2022. The entire contents of the foregoing are incorporated herein by reference.

### FEDERALLY SPONSORED RESEARCH OR DEVELOPMENT

This invention was made with Government support under Grant Nos. AG006173, AG015379, AG071865, and AG063046 awarded by the National Institutes of Health. The Government has certain rights in the invention.

### TECHNICAL FIELD

Described herein are compositions and methods for making and using antibodies that bind specifically to soluble oligomers of amyloid beta protein (oAβ), and methods of use thereof in diagnosis and treatment of diseases related to formation of pathogenic oligomers of oAβ. Also provided are T helper cell (Th) carrier peptides that can be conjugated to peptide immunogens to stimulate targeted humoral immune responses to the desired antigen.

### BACKGROUND

Alzheimer's disease (AD) is a slowly progressive neurodegenerative disorder at both the histopathological and clinical levels and is the cause of around 70% of all dementias. Early symptoms of episodic mild memory loss and minimal intermittent cognitive impairment lead gradually over 5-15 years to profound dementia and death. An estimated 6.2 million Americans and over 45 million people worldwide are currently living with AD, causing an enormous familial, societal, and economic burden. AD is the fifth-leading cause of death among senior Americans and in 2020, unpaid care for patients with dementia was valued at $256.7 billion (2021 Alzheimer's facts and figures). Prior to aducanumab's approval in June of 2021, there were only five drugs approved for treating AD—four cholinesterase inhibitors and one N-methyl-D-aspartate (NMDA) receptor antagonist—but these are only symptomatic treatments with minimal effect, none of these drugs treat the underlying cause of AD and do not slow cognitive decline (Cummings et al, 2016). Aducanumab, sold under the brand name Aduhelm, is the first disease-modifying therapy available to treat Alzheimer's disease. Aduhelm has high specificity for insoluble Aβ aggregates and has been shown to reduce brain Aβ plaque load in a time- and dose-dependent manner (Arndt et al, 2018; Sevigny et al, 2016). The approval adds important clinical support to the amyloid hypothesis and validates Aβ as a drug target, but despite Aduhelm's positive target engagement and ability to reduce plaque load, serious questions remain on the drug's effect on cognitive decline (Knopman et al, 2021). Aduhelm's approval is a boon for patients and families struggling with AD and highlights the urgent need to develop more effective and affordable diagnostic strategies and disease-modifying therapies for AD.

### SUMMARY

Provided herein are compositions and methods for making and using antibodies that bind specifically to soluble oligomers of amyloid beta protein (oAβ), as well as the antibodies themselves, and methods of use thereof in diagnosis and treatment of diseases related to formation of pathogenic oligomers of oAβ. Also provided are T helper cell (Th) carrier peptides that can be conjugated to peptide immunogens to stimulate targeted humoral immune responses to the desired antigen.

Thus provided herein synthetic peptide immunogen for stimulating development of antibodies, wherein the synthetic peptide immunogen is specific to conformational epitopes present in an aggregated form of the amyloid β protein (Aβ) comprising oligomers of amyloid beta protein (oAβ), but not the monomeric form. In some embodiments, the synthetic peptide immunogen comprises sequence GYEVHHQKLV (SEQ ID NO:1), or a sequence YEXHH (SEQ ID NO:41), wherein X is a hydrophobic amino acid (V, I, or L).

In some embodiments, the synthetic peptide immunogen comprises sequence CGKCGYEVHHQKLVPNVLKQHHVEYGCK (SEQ ID NO:2) or CGKCGYEVHHQKLVPNGYEVHHQKLVCK (SEQ ID NO:3). In some embodiments, the synthetic peptide immunogen comprises a sequence as shown in FIG. 13, i.e., SEQ ID NO:5-17. In some embodiments, the synthetic peptide immunogen comprises a sequence of peptide 1, 3, 5, 7, 8, 11, or 13, preferably peptide 5, 8, or 7 being the best.

In some embodiments, the synthetic peptide immunogen further comprises a carrier protein linked to N terminus, optionally wherein the carrier protein is linked by maleimide. In some embodiments, the synthetic peptide immunogen comprises the carrier protein comprises Keyhole Limpet Hemocyanin (KLH), bovine serum albumin (BSA), or ovalbumin (OVA).

In some embodiments, the synthetic peptide immunogen comprises the carrier protein comprises a peptide having the sequence KSSKSKKKFISEAIIHHLHSRHPGK (SEQ ID NO:4), optionally with (i) a Pam3Cys (N-α-Palmitoyl-S-2,3-bis(palmitoyloxy)-(2RS)-propyl-L-cysteine, Palmitoyl-Cys((RS)-2,3) attached to the second lysine (K4), wherein the carrier protein is conjugated to the N terminus via maleimide.

Also provided herein are synthetic peptides having the sequence KSSKSKKKFISEAIIHHLHSRHPGK (SEQ ID NO:4), optionally with (i) a Pam3Cys (N-α-Palmitoyl-S-2,3-bis(palmitoyloxy)-(2RS)-propyl-L-cysteine, Palmitoyl-Cys((RS)-2,3) attached to the second lysine (K4), and methods of use thereof for increasing an immune response to an antigen.

Further provided are compositions comprising the peptides described herein.

Additionally, provided herein are methods for detecting oAβ in a sample comprising one or more biological fluids, preferably a sample from a human subject. The methods comprise contacting the sample with capture antibodies bound to a surface, wherein the capture antibodies comprise 71A1 or 1G5 antibodies, under conditions sufficient for formation of capture antibody-oAβ complexes, optionally washing to remove unbound proteins; contacting the sample with a labeled detection antibody comprising a detectable label, wherein the detection antibody binds to the capture antibody-oAβ complexes, under conditions sufficient for the detection antibody to bind to the capture antibody-oAβ complexes; and detecting and optionally quantifying the detectable label, wherein the amount of detectable label is proportional to the amount of oAβ in the sample, thereby detecting oAβ in the sample.

In some embodiments, the one or more biological fluids comprise whole blood, plasma, serum, urine, saliva, breath, exosome or exosome-like microvesicles, lymph, cerebrospinal fluid, or sputum blood or plasma. Tissue can also be used. Preferably, the sample is whole blood, plasma or serum. In some embodiments, the sample is diluted; e.g., a sample comprising plasma is diluted 1:2 to 16 times, e.g., 1:4 to 1:8 times; a sample comprising CSF can be diluted 1:10 to 1:20 times, e.g., 1:12-1:18, e.g., 1:16 times.

In some embodiments, the surface is the surface of a plate or a bead. In some embodiments, the plate is a multiwell plate (e.g., a 96 well plate) or a test strip. In some embodiments, the bead is selected from magnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, or TEFLON® beads.

In some embodiments, the capture antibody is conjugated to a biotin moiety, the surface is coated with streptavidin, and the antibody is attached to the surface via biotin-streptavidin binding.

In some embodiments, the detection antibody binds to Aβ at a different epitope from the capture antibody.

In some embodiments, the capture antibody comprises an affinity tag, and the detection antibody binds to the affinity tag.

In some embodiments, the detectable label is a radioisotope, a chemiluminescent substance, a fluorescent substance, a metal complex, a bioluminescent substance such as a luciferase, or a nucleic acid.

In some embodiments, detecting and optionally quantifying the concentration of oAβ in the sample comprises using a radio immunoassay method (RIA) when the detection antibody is labeled with a radioisotope, a chemiluminescent immunoassay method (CIA) when the detection antibody is labeled with a chemiluminescent substance, a fluorescent immunoassay method (FIA) when the detection antibody is labeled with a fluorescent substance, an electro-chemiluminescent immunoassay method (ECLIA) when the detection antibody is labeled with a metal complex, a bioluminescent immunoassay method (BLIA) when the detection antibody is labeled with a bioluminescent substance such as a luciferase, an immuno-PCR comprising amplifying a nucleic acid that labeled an antibody by PCR and detecting the nucleic acid, when the detection antibody is labeled with a nucleic acid, a turbidimetric immunoassay method (TAI) detecting turbidity occurred by forming an immunocomplex, a latex agglutination turbidimetric assay (LA) comprising detecting latex aggregated by formation of complexes, or an immunochromatography assay utilizing a reaction on a cellulose membrane.

Additionally, provided herein are kits comprising: a surface, optionally a plate or beads; a capture antibody selected from 71A1 or 1G5, optionally linked to the surface; a detection antibody; and a standard comprising a known concentration of oAβ. In some embodiments, the surface is the surface of a plate or a bead. In some embodiments, the plate is a multiwell plate or a test strip.

In some embodiments, the bead is selected from magnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, or TEFLON® beads.

In some embodiments, the capture antibody is conjugated to a biotin moiety, the surface is coated with streptavidin, and the antibody is attached to the surface via biotin-streptavidin binding.

In some embodiments, the detection antibody binds to Aβ at a different epitope from the capture antibody.

In some embodiments, the capture antibody comprises an affinity tag, and the detection antibody binds to the affinity tag.

In some embodiments, the detectable label is a radioisotope, a chemiluminescent substance, a fluorescent substance, a metal complex, a bioluminescent substance such as a luciferase, or a nucleic acid.

Also provided herein are methods for treating a neurological disorder associated with accumulation of oAβ in a subject, the method comprising administering a therapeutically effective amount of 71A1 antibody, or an antigen-binding fragment thereof, or 1G5 antibody, or an antigen-binding fragment thereof to the subject. Also provided are the 71A1 antibody, or an antigen-binding fragment thereof, or 1G5 antibody, or an antigen-binding fragment thereof, e.g., for use in methods for treating a neurological disorder associated with accumulation of oAβ in a subject, and compositions comprising the 71A1 antibody, or an antigen-binding fragment thereof, or 1G5 antibody, or an antigen-binding fragment thereof. In some embodiments, the disorder is cerebral amyloid angiopathy (CAA) or Alzheimer's disease (AD). In some embodiments, the antibody is administered to the brain of the subject. In some embodiments, the antibody is a monoclonal antibody, a humanized antibody, or a chimeric antibody.

Provided herein are quantitative methods for assaying a target analyte, present in one or more biological fluids. The methods are comprised of: (i) obtaining a fluid sample from a human patient; (ii) capturing said target analyte, present in said fluid, with a first agent or probe (the capture probe) that recognizes and isolates said target analyte from said fluid; and (iii) quantifying the concentration of said analyte in said biological fluid using a second agent or probe (the detector probe), conjugated with a detector signal, which recognizes a different epitope on said target analyte, and wherein said target analyte is quantified by said detector signal. In some embodiments, the target analyte is a soluble neurotoxic oligomeric antigen (oAβ) derived from the amyloid β-protein (Aβ) In some embodiments, oAβ is comprised of Aβ peptide isoforms. In some embodiments, the Aβ peptide isoforms are Aβ peptides naturally secreted in biological fluids. In some embodiments, said naturally secreted Aβ peptides include Aβ 37, As38, As39. Aβ40, Aβ42, As43 and any N-terminal variants thereof. In some embodiments, the capture probe and the detector probe include monoclonal antibodies, derivatives of monoclonal antibodies, modifications of monoclonal antibodies, or polyclonal antibodies. In some embodiments, said capture monoclonal antibody is oAβ-selective and recognizes and binds soluble oligomers comprised of any of the Aβ peptides described herein. In some embodiments, the capture monoclonal antibody is 71A1 or 1G5. 9. In some embodiments, the capture probe or monoclonal antibody is conjugated with a complementary binding molecule. In some embodiments, the complementary binding molecule includes, but is not limited to, biotin. In some embodiments, the capture probe is bound to a substrate or surface. In some embodiments, the substrate or surface is coated with streptavidin. In some embodiments, the substrate or surface for the capture probe includes beads, particles, microspheres, nanotubes, polymers, plates, disks, or dipsticks. In some embodiments, the type of bead may be characterized as magnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, or TEFLON beads. In some embodiments, the detector probe or monoclonal antibody recognizes an N-terminal epitope of Aβ. In some embodiments, the detector probe or monoclonal antibody includes, but is not limited to, 3D6. In some embodiments, the detector probe or monoclonal antibody is conjugated with, but not limited to, an enzymatic label, a dye, a fluorescent label or a metal label. In some embodiments, the fluorescent label is a bright, far-red-fluorescent dye with excitation ideally suited for the 594 nm or 633 nm laser lines. In some embodiments, the fluorescent label is measured using a confocal microscope lens and a photo detector.

Also provided herein are non-invasive methods to screen a human patient for the existence and course of a neurological disease or condition, characterized by the deposition and/or production of soluble neurotoxic Aβ oligomers, comprised of: (i) quantifying the amount of soluble neurotoxic Aβ oligomeric antigen in a bodily fluid from said human patient using a method as described herein; (ii) comparing the quantity of soluble neurotoxic Aβ oligomers in said human patient sample with the quantity of soluble neurotoxic Aβ oligomers in fluid samples isolated from a normal human reference population wherein; (iii) a change in the quantity of the soluble neurotoxic Aβ oligomers present in the fluid sample of said human patient, relative to the quantity of the soluble oligomer present in the fluid samples of said normal human reference population is a biomarker or diagnostic for the presence and/or course of said neurological disease or condition. In some embodiments, the non-invasive method screen is a biomarker for the effectiveness of a therapeutic intervention for said neurological disease or condition.

Further provided herein are kits for quantifying the amount of soluble neurotoxic Aβ oligomeric antigens in a sample obtained from a human patient comprised of; (i) capture and detector reagents as described herein and (ii) instructions for use of said reagents to quantify soluble neurotoxic Aβ oligomeric antigens in said human patient sample.

Additionally, provided herein are methods to prevent, slow the progress of, or treat a disease characterized, partially or wholly, by the pathological extracellular deposition of amyloid in a patient comprised of the administration to said patient of one or more therapeutically effective doses of an agent that inhibits neurotoxic oligomers of amyloid beta protein (oAβ). In some embodiments, the agent is a monoclonal antibody. In some embodiments, the monoclonal antibody is 71A1 or 1G5. In some embodiments, the agent is administered by means of, but not limited to, the following routes: oral, sublingual, parenteral, intraperitoneal, intramuscular, intravenous, topical, intraocular, intranasal, intracerebral, intraventricular, and intracisternal. In some embodiments, the disease is a neurodegenerative disease, e.g., Alzheimer's Disease.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of an exemplary peptide immunogen, also referred to herein as NETL-1. In this example, the peptide is shown conjugated to a maleimide carrier.

FIG. 2 is a standard, indirect enzyme linked immunosorbent assay (ELISA) with the purified monoclonal antibodies assayed for binding to Aβ40, NETL 1 or NETL 2 peptides conjugated to malemide-activated (SH-reactive) micro titer plates.

FIGS. 3A-D. 1G5 and 71Aβ pull down Aβ from human brain soaking extract. (A) Schematic illustration of preparation of human brain soaking extracts. (B) immunoblots of immunoprecipitation with protein G by different antibodies from human brain soaking extract, detected by 2 antibodies against middle region and N-terminal of Aβ. 7.5 μL of the extract was used for the input lane and IP was from 800 μL of extract. (C) Aβ x-40 and x-42 measured by ELISA of immunoprecipitation or post-immunoprecipitation supernatant (all denatured in 8M GnCl) by the specified antibodies from human brain soaking extracts, n=3, means±SD.

FIGS. 4A-D.1G5 and 71A1 recognize Aβ in human brain in situ. (A) Immunohistochemistry using 1C22, 1G5, and 71A1 on human brain cryo-sections; scale bar=200 μm. (B) Immunohistochemistry using 1C22 or 71A1 on human brain cryo-sections, with double labeling by Aβ monomer antibody D54D2; bar=200 μm for left two panels and 100 μm for right panel. (C) LTP induction after treatment with aCSF (n=6), human brain soaking extract (n=5), 2.12 ug/mL 71A1 (n=4), or human brain soaking extract premixed with 2.12 ug/mL 71A1 (n=4), means±SD. (D) LTP induction after treatment with aCSF (n=6) or 71A1 affinity purified oAβ (n=4), mean±SD.

FIGS. 4E-F. Statistical test of electrophysiological recordings on hippocampal slices. (E) Multiple t-test analysis of (FIG. 4C) human brain soaking extracts treatment vs. the same extract premixed with 2.12 ug/mL of 71A1 treatment: q value distribution throughout the 60 min recording. (F) multiple t-test analysis of (FIG. 4D) between aCSF treatment vs. 71A1-purified oAβ treatment; q value distribution throughout the 60 min recording.

FIGS. 5A-C. 1G5 and 71A1 recognize Aβ from human CSF. (A) Aβ x-40 and x-42 measured by ELISA of immunoprecipitates or post-immunoprecipitation supernatants (all denatured by 8M GnCl) by different antibodies from 3 human CSFs, with technical replicates=3, means±SD. (B) Aβ x-40 and x-42 measured by ELISA of immunoprecipitatees or post-immunoprecipitation supernatants (all denatured by 8M GnCl) by 71A1 from 19 individual human CSFs, technical replicates=3, means f SD. (C) correlation between Aβ x-40 and x-42 measured by ELISA of immunoprecipitates (all denatured by 8M GnCl) by 71A1 and the ADmark Aβ 1-42 measurements from the same CSFs. Pearson correlation is used.

FIGS. 6A-I. Development of 71A1/3D6 immunoassay specific for oAβ. (A) Schematic illustration of the SMCxPRO beads-based immunoassay. (B-C) 1G5/3D6 and 71A1/3D6 assay performance on ADDLs as calibrator; (D) average CV and recovery of ADDLs signals using 1G5/3D6 and 71A1/3D6 assays. (E) 1C22/3D6 and 71A1/3D6 assay performance on ADDLs as calibrator. (F) CBB staining of PAGE analysis of Aβ1-40 S26 dimer without (−) or with (+) reduction by DTT. (G-H) 1C22/3D6 and 71A1/3D6 assay performance on Aβ1-40 S26 dimer without (native) or with reduction by DTT. (I) 71A1/3D6 assay performance on human brain soaking extract: left panel: raw value of 71A1/3D6 signals from serial diluted brain extract calibrated by ADDLs; middle panel: calculated value adjusted by dilution factors; right panel; recovery of each dilution normalized to 1:1K; n=3, mean±SD.

FIGS. 7A-D. 71A1/3D6 immunoassay recognizes high molecular weight oAβ from human brain. (A-B) Chromatogram (UV280 nm absorbance) of molecular weight calibrators and human brain soluble extract fractioned by Superdex 200 increase SEC column. (C) oAβ measured by 71A1/3D6 immunoassay from SEC fractions natively; n=3, mean±SD. (D) monomeric Aβ x-42 measured by MSD ELISA from SEC fractions natively (red bars) or denatured (blue bars), n=2, mean±SD.

FIGS. 8A-F. 71A1/3D6 immunoreactive oAβ in CSF correlates with t-Tau and p-Tau levels. (A) 71A1/3D6 assay performance on human CSF: left panel: raw values of 71A1/3D6 signals from serially diluted CSF (against ADDLs calibrator); middle panel: calculated value adjusted by dilution factors; right panel: recovery of each dilution normalized to 1:2; n=3, means SD. (B-D) Correlations between 71A1/3D6 reactive oAβ levels in CSF and ADmark measurements of Aβ 1-42, t-Tau and p-Tau; n=29. Pearson correlation is used. (E) Chromatogram (UV280 nm absorbance) of human CSF fractioned on a Superdex 200 Increase SEC column. (F) oAβ measured by the 71A1/3D6 immunoassay in SEC fractions natively; n=3, mean±SD.

FIGS. 9A-G. 71A1/3D6 immunoassay accurately quantifies oAβ from human plasma. (A) 71A1/3D6 assay performance on human plasma: left panel: raw value of 71A1/3D6 signals from serially diluted plasma against ADDLs calibrator; middle panel: calculated values adjusted by dilution factors; right panel: recovery of each dilution normalized to 1:4; n=3, means±SD. (B) spike-in recovery test by spiking human brain homogenate (Brain H), human brain soaking extract (Brain S) or human CSF into 8-fold diluted individual plasma; n=3, means±SD. (C) 71A1/3D6 assay of oAβ from 8-fold diluted individual plasmas immunodepleted by different antibodies, compared to the input plasma; n=4. (D) 71A1/3D6 assay of oAβ from human brain soaking extract, CSF and plasma denatured by 8M GnCl (or not); n=3, means±SD. (E) 71A1/3D6 assay of oAβ from 73 human plasmas (Mayo Aging cohort), n=4. (F) Raw values of 71A1/3D6 signals from serially diluted plasma (against ADDLs calibrator); n=3, mean±S.; (G) Recovery of each dilution normalized to the average of all dilutions; n=3, mean±SD.

FIGS. 10A-C. A. Quantification of oAβ in APP NLGF/NLGF mouse brain soaking extracts demonstrates age dependent accumulation of 71A1 positive oAβ in mouse brain. B. Measurements of Long-term potentiation (LTP) on 6 month old APP NLGF/NLGF mouse and age matched wild-type mouse hippocampus demonstrates LTP impairment in 6 month old APP NLGF/NLGF mouse. C. Soaking extracts from 6 month old APP NLGF/NLGF mouse was used to treat wild-type mice hippocampus followed by LTP measurements. 6 month old APP NLGF/NLGF mouse brain extracts could impaired the LTP of the treated wild-type mouse hippocampus. And mixture of 71A1 in the treatment will rescue the impairment caused by 6 month old APP NLGF/NLGF mouse brain extracts.

FIGS. 11A-B. A. Y-maze performance of 71A1 or control antibody treated mice, which is demonstrated by both spontaneous alteration and arm entries. 71A1 treatment significantly improved performance of male mice but not female compared to control antibody treatment. B. 71A1 treatment decreased oAβ amount in brain of male mice (trend without statistical significance).

FIGS. 12A-B. A. Schematic of LTP measurement of wild-type mouse hippocampus treated with brain extracts from antibody trial APP NLGF/NLGF mice. B. Wild-type mouse hippocampus was treated with brain extracts from 71A1 treated (n=3) and control antibody treated (n=2) male mice, to demonstrate 71A1 treatment reduced the synaptotoxicity of APP NLGF male mouse brain, reflected by failure to inhibit LTP of wild-type mice hippocampus.

FIG. 13. 12-amino acid sequences that are synthetic peptide mimetics, which bind to Mab 71A1, isolated using New England BioLab's Ph.D.-12™ Phage Display Peptide Library Kit. SEQ ID NOs. 5-17 appear in order.

FIGS. 14A-B. ELISA with purified monoclonal antibody 71A1 assayed for binding to peptide mimetics (A) 1-7 and (B) 8-13, as shown in FIG. 13, conjugated to maleimide-activated micro titer plates.

### DETAILED DESCRIPTION

Biochemical and morphological studies suggest that clinical impairment in AD involves early synaptic dysfunction (Tu et al, 2014; Anderton et al., 1998; Cummings et al., 1998), followed by more severe neuronal changes that include increased synaptic loss, widespread neuritic dystrophy, neurofibrillary tangles and frank neuronal death (Serrano-Pozo et al, 2011; Tonnies & Trushina, 2017; Terry, 1963; Gomez-Isla et al., 1996; Sze et al., 1997; Anderton et al., 1998). The mechanism underlying the initiation of this progressive pathophysiology is thought to involve the age-related dysregulation and accumulation of the amyloid β-protein (Aβ) and the subsequent hyperphosphorylation and aggregation of the microtubule-associated protein tau, both of which can be observed in fibrillar-rich neuritic plaques and neurofibrillary tangles, respectively, upon autopsy (Braak & Braak, 1996; Perl, 2010; Esiri et al., 1997). The observation of these end-stage lesions in postmortem brain tissue and the appearance of amyloid plaques before neurofibrillary tangles has led to an assumption that accumulation of fibrils per se underlies the progression of AD (Serrano-Pozo et al, 2011). This impression has been supported in part by studies of primary neuronal cultures, in which progressive neurodegeneration can be induced by highly aggregated, fibrillar Aβ but not by equivalent concentrations of Aβ monomers (Mattson et al., 1993b; Pike et al., 1993; Lorenzo et al., 1994). Furthermore, several studies indicate that Aβ dysregulation occurs before and likely induces tau hyperphosphorylation, eventually leading to clinical manifestations of disease (Zheng et al 2002; Wu et al, 2018; Bloom 2014; Viola and Klein, 2015).

A hallmark of Alzheimer's disease (AD) is the formation of fibril-rich plaques that are composed of amyloid b-protein (Aβ) and that were thought to disrupt normal communication between brain cells, eventually leading to neuronal cell death. The accumulation of amyloid plaques was once thought solely responsible for the nerve cell toxicity found in Alzheimer's disease, but small, soluble aggregates of beta-amyloid may be more toxic (Hong et al, 2018; Cline et al, 2018).

Aβ is a peptide fragment of a larger protein called amyloid precursor protein or APP. A portion of APP is expressed on the surface of neurons and subsequently this portion of it is cleaved to produce a 40 or 42 amino acid fragment called Aβ. Oligomeric Aβ assemblies and larger intermediates, referred to as protofibrils (PFs), are formed during the process of fibril generation. In vitro studies have shown that soluble oligomeric species of Aβ and protofibrils can alter neurophysiology and can be neurotoxic, strongly suggesting that these assemblies may play a role in the pathophysiology of AD (Cleary et al, 2005; Hong et al, 2018; Shankar et al, 2008; O'Malley et al, 2016). The development of antibodies that could specifically recognize oligomeric forms of Aβ would be invaluable in understanding the central role they play in the initiation of brain dysfunction and injury. Specifically, these antibodies would be useful in identifying when these assemblies arise and how they may correlate with the memory impairments and cognitive decline in transgenic mice and AD patients. Additionally, if protofibrils or unique, small aggregates of Aβ form in humans, these novel antibodies can be valuable in identifying these assemblies in blood and/or tissues, helping to diagnose early stages of AD.

Aβ is thought to start accumulating in vivo as low molecular weight species (LMW Aβ) consisting principally of monomers that are constitutively secreted from brain cells (Haass et al., 1992; Shoji et al., 1992). These may, under certain circumstances, progress to oligomers and ultimately to mature 7-10 nm wide amyloid fibrils, as observed in mature plaques. Aβ oligomers (dimers, trimers, tetramers and possibly larger assemblies) have been identified in the conditioned media of certain cell lines overexpressing APP that constitutively secrete Aβ (Podlisny et al., 1995; Xia et al., 1997; Podlisny et al., 1998), and in cerebrospinal fluid in AD patients (Pitschke et al., 1998; Walsh et al., 2000). Recent data from several labs also suggest that oligomeric species formed during Aβ fibrillogenesis may contribute to neuronal injury (Hong et al, 2018; Li et al, 2018). For example, an Aβ complex sedimenting more slowly than amyloid fibrils was shown to have a neurotoxic effect on cultured PC12 cells, as quantified by the MTT conversion assay (Oda et al., 1995). In addition, β-1-antichymotrypsin is capable of blocking fibril formation without decreasing Aβ-induced neurotoxicity (Aksenova et al., 1996). A soluble Aβ oligomeric species isolated from AD cerebral cortex that kills neurons in mixed brain cultures has also been described (Giulian et al., 1996; Roher et al., 1996). Furthermore, a recently described soluble form of synthetic Aβ referred to as Aβ derived diffusible ligands (ADDLs) are neurotoxic in vitro (Lambert et al., 1998). In support of this prefibrillar concept, transgenic mice over-expressing human APP show altered behavior and/or electrophysiological changes prior to any substantial Aβ deposits or neuronal pathology being observed (Holcomb et al., 1998; Chapman et al., 1999; Hsia et al., 1999). These data clearly suggest that prefibrillar forms of Aβ may cause synaptic dysfunction and could thus contribute to early memory loss and mild cognitive changes in the initial stages of AD. Interestingly, a recent human study found that the best correlation between mental status and synaptic loss in AD patients was the amount of soluble Aβ. However, the biophysical characteristics of the soluble Aβ species were not determined (Lue et al., 1999).

As described above, fibril formation is believed to proceed via a transition of LMW Aβ to metastable intermediate species that go on to form fibrils (Harper et al., 1997a; Teplow, 1998). Two laboratories have identified such intermediates in the formation of synthetic Aβ fibrils, and they are referred to as protofibrils (PFs) (Harper et al., 1997a; Harper et al., 1997b; Walsh et al., 1997). Labs have investigated the electrophysiology and neurotoxicity of both low molecular weight Aβ (LMW) (i.e. monomers/dimers) and PFs and their relationships to fibrillar Aβ (Hartley et al., 1999). It was found that both of these earlier species reproducibly induce toxicity in cultured primary cortical neurons over a period of days. This toxicity was not associated with detectable appearance of amyloid fibrils, as measured by Congo red binding and immuno-electron microscopy (Hartley et al., 1999). Consistent with PFs having intrinsic biological activity, they actively and reproducibly increased excitatory postsynaptic currents (EPSCs), excitatory postsynaptic potentials (EPSPs), action potentials (APs), and membrane depolarizations (MDs) (Hartley et al., 1999). Importantly, it was found that PFs have such effects at doses as low as 50-100 nM, whereas monomeric Aβ prepared and applied simultaneously at the same concentrations elicits no electrophysiological response. These effects of PFs were entirely reversible within 65 min after application (see Ye et al., 2000).

Elucidating the biological activity of PFs should help to determine the role of early Aβ intermediates in the mechanism of neuronal dysfunction in AD, with attendant therapeutic implications. Interestingly, the aggregation of other proteins may play an important role in Parkinson's, Prion diseases, polyglutamine expansion disorder like Huntington's disease, and frontotemporal dementia (Kim et al., 1999; Lansbury et al., 2000). Thus, understanding the details of early Aβ aggregation and consequent neuronal dysfunction may serve as a prototype for understanding other brain diseases involving protein aggregation. Therefore, reagents, such as protofibril specific antibodies, that are critical for identifying the pathological species would be invaluable for determining their role in these neurodegenerative diseases.

Studies of amyloid protofibrils and fibrils show a characteristic β-sheet structure with polypeptide chains running roughly perpendicular to the long axis of the fibril and interchain hydrogen bonds parallel to the axis. It is the amyloid fibrils that form the ‘senile plaques’ in Alzheimer's disease. How fibrils form and the exact nature of the early events that led to fibril formation is poorly understood. β-sheet intermediates of Aβ peptides, often referred to as protofibrils, form during the process of fibrillogenesis. An ability to immunologically detect these structures early in the onset of disease might be useful in determining their role in the pathogenesis of AD. Moreover, if these early assemblies are responsible, at least in part, for the initiation of AD, then they could possibly be used as a diagnostic marker and may contribute to the development of therapeutic agents that target these structures. Antibodies that detect protofibrils would be highly valuable in this endeavor and would be useful for monitoring therapeutic efficacy. Multiple studies have suggested that passive immunization of animal models of AD with an anti-Aβ antibody can reduce Aβ burden and can reverse behavioral deficits (Morgan, 2011). This would indicate that passive immunization with an anti-Aβ antibody directed to an early pathologic species would be of significant value for early intervention in AD.

### Peptide Immunogens

Described herein are synthetic peptide immunogens, e.g., peptides that are optionally 6-50 amino acids long, e.g., at least 6, 7, 8, 9, 10, 11, or 12 amino acids, up to 15, 20, 25, 30, 35, 40, 45, or 5, and the structure and sequence of a region within the Aβ peptide that can be used to stimulate the development of antibodies that bind to Aβ aggregates and not to the normally occurring monomeric Aβ amino acid peptide.

In some embodiments, the synthetic peptide immunogen comprises the Aβ peptide sequence GYEVHHQKLV (SEQ ID NO:1).

In some embodiments, the synthetic peptide immunogen comprises the peptide sequence:

(SEQ ID NO: 2)
CGKCGYEVHHQKLVPNVLKQHHVEYGCK.

The peptide immunogens are useful, e.g., for generating isolated and purified antibodies including, but not limited to, IgG subclass 1 monoclonal antibodies 71A1 and 1G5.

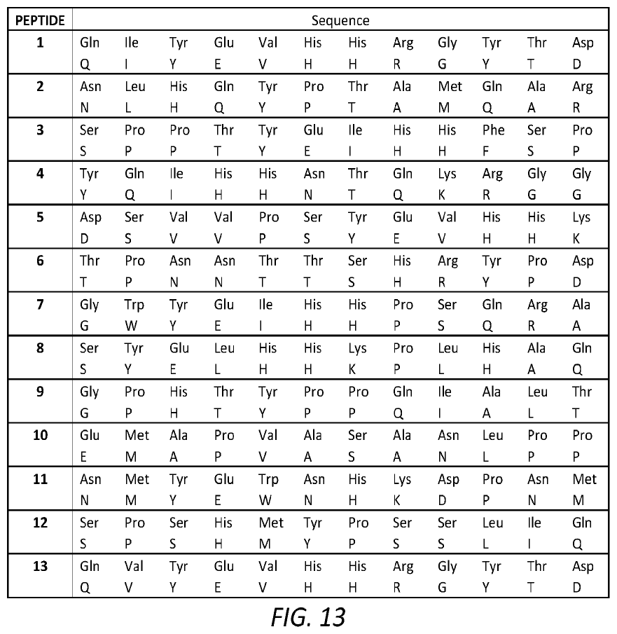

Additionally provided herein are peptide immunogens comprising peptide mimetic sequences that bind to the 71 A1 variable region. These peptide mimetics are linear peptide sequences that mimic the part of the 3-dimensional structure of the Abeta oligomer that is specific for mab 71 A1 or 1g5, and thus mimic the native antigenic structure with a linear sequence that also binds to the antibodies (See FIG. 13).

These peptide mimetics were affinity selected using monoclonal antibody 71A1 and a commercially available phage display library kit (see Example 6). Thus also provided are peptides comprising a sequence as shown in FIG. 13, e.g., one of SEQ ID NOs:5-17.

TABLE A
PEPTIDE MIMETICS

| PEPTIDE NUMBER | SEQUENCE | SEQ ID NO: |
|---|---|---|
| 1 | QIYEVHHRGYTD | 5 |
| 2 | NLHQYPTAMQAR | 6 |
| 3 | SPPTYEIHHFSP | 7 |
| 4 | YQIHHNTQKRGG | 8 |
| 5 | DSVVPSYEVHHK | 9 |
| 6 | TPNNTTSHRYPD | 10 |
| 7 | GWYEIHHPSQRA | 11 |
| 8 | SYELHHKPLHAQ | 12 |
| 9 | GPHTYPPQIALT | 13 |
| 10 | EMAPVASANLPP | 14 |
| 11 | NMYEWNHKDPNM | 15 |
| 12 | SPSHMYPSSLIQ | 16 |
| 13 | QVYEVHHRGYTD | 17 |

In some embodiments, the peptide immunogen comprises a sequence YEXHH (SEQ II) NO 41), wherein X is a hydrophobic amino acid (V, I, or L). In some embodiments, the peptide immunogen comprises a sequence GYEVHHQKLV (SEQ ID NO 1), or a sequence that is at least 80, 85, 90, 95, or 990% identical to that sequence, e.g., comprises a mutation at 1, 2, or 3 amino acids.

The peptides and peptide immunogens of this invention can be made by chemical synthesis methods that are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the alpha-NH₂ protected by either t-Boc or F-moc chemistry using side chain protected amino acids on, for example, an Applied Biosystems Peptide Synthesizer Model 430A or 431. Preparation of peptide constructs comprising combinatorial library peptides for Th epitopes can be accomplished by providing a mixture of alternative amino acids for coupling at a given variable position. After complete assembly of the desired peptide immunogen, the resin is treated according to standard procedures to cleave the peptide from the resin and deblock the functional groups on the amino acid side chains. The free peptide is purified by HPLC and characterized biochemically, for example, by amino acid analysis or by sequencing. Purification and characterization methods for peptides are well known to one of ordinary skill in the art.

The peptides and peptide immunogens can be used, e.g., to elicit an immune response in an animal, e.g., to generate antibodies that bind to Aβ, e.g., Aβ aggregates (oAβ). Methods for making antibodies are known in the art and described herein. Also provided herein are methods for using the peptides and peptide immunogens described herein, and compositions comprising the peptides and peptide immunogens described herein. In some embodiments, the compositions comprise an adjuvant; in some embodiments, the adjuvant is covalently linked to the peptide. In some embodiments, the peptide is covalently linked to a carrier comprising Tepi-2 (SEQ ID NO:4), or to Keyhole Limpet Hemocyanin (KLH), bovine serum albumin (BSA), or ovalbumin (OVA).

### Antibodies 71A1 and 1G5

As described above, the present inventors succeeded in obtaining antibodies that bind specifically to Aβ oligomers but not to Aβ monomers. That is, the present disclosure provides antibodies that bind to Aβ oligomers but not to Aβ monomers.

Antibodies useful in the present methods and compositions include 71A1 and 1G5, and variants and derivatives thereof.

Antibody 71A1
Heavy Chain:
DNA Sequence
(SEQ ID NO: 18)
GAGGTGCAGCTGCAGGAGTCTGGGGCTGAACTGGTGAGGCCTGGGGTTTC
AGTGAAGCTGTCCTGCAAGACTTCTGACTACACATTCACCAGTTATTGGTT
GCACTGGTTTAAGCAGAGGCCTGAACAAGGCCTTGAGAGGATTGGAGAGA
TTGATCCTAGCAATGGTGATGCTGACTACAGTGAGAAGTTCAAGAGCAAG
GCCACACTGACTGTAGACAAATCTTCCACCACAGCCTACATGCAGCTCGCC
AGTCTGACATCTGAGGACTCTGCGGTCTATTACTGTGCAAGAGAAGCCTAC
GGCCACTACTTTGACTACTGGGGCCAAGGCACCACTCTCACAGTCTCCTCA
G
Predicted Protein Sequence (complementarity determining regions (CDRs)
are in bold)
(SEQ ID NO: 19)
EVQLQESGAELVRPGVSVKLSCKTSDYTFTSYWLHWFKQRPEQGLERIGEIDP
SNGDADYSEKFKSKATLTVDKSSTTAYMQLASLTSEDSAVYYCAREAYGHY
FDYWGQGTTLTVSS
HC CDR1:
(SEQ ID NO: 20)
DYTFTSYW
HC CDR2:
(SEQ ID NO: 21)
IDPSNGDA
HC CDR3:
(SEQ ID NO: 22)
AREAYGHYFDY
Light Chain:
DNA Sequence
(SEQ ID NO: 23)
GATGTTGTGATGACCCAAACTCCACTCTCCCTGCCTGTCAGTCTTGGAGAT
CAAGCCTCCATCTCCTGCAGATCCAGTCAGAGCCTTGAACACAGTAATGG
AAACACCTATTTACATTGGTACCTGCAGAAGCCAGGCCAGTCTCCAAAGCT
CCTGATCTACAAACTTTCCAACCGATTTTCTGGGGTCCCAGACAGGTTCAG
TGGCAGTGGATCAGGGACAGATTTCACACTCAAGATCAGCAGAGTGGAGG
CTGAGGATCTGGGAGTTTATTTCTGCTCTCAAAGTACACATGTTCCGTACA
CGTTCGGAGGGGGGACCAAGCTGGAAATAAAAC
Predicted Protein Sequence (complementarity determining regions (CDRs)
are in bold)
(SEQ ID NO: 24)
DVVMTQTPLSLPVSLGDQASISCRSSQSLEHSNGNTYLHWYLQKPGQSPKLLI
YKLSNRFSGVPDRFSGSGSGTDFTLKISRVEAEDLGVYFCSQSTHVPYTFGGG
TKLEIK
LC CDR1:
(SEQ ID NO: 25)
QSLEHSNGNTY
LC CDR2:
(SEQ ID NO: 26)
KLS
LC CDR3:
(SEQ ID NO: 27)
SQSTHVPYT
Antibody 1G5
Heavy Chain:
DNA Sequence
(SEQ ID NO: 28)
CAGGTCCAACTGCAACAGCCTGGGGCTGAACTGGTGAGGCCTGGGGTTTC
AGTGAAGCTGTCCTGCAAGACTTCTGACTACACATTCACCAGTTATTGGTT
GCACTGGTTTAAGCAGAGGCCTGAACAAGGCCTTGAGAGGATTGGAGAGA
TTGATCCTAGCAATGGTGATGCTGACTACAGTGAGAAGTTCAAGAGCAAG
GCCACACTGACTGTAGACAAATCTTCCACCACAGCCTACATGCAGCTCGCC
AGTCTGACATCTGAGGACTCTGCGGTCTATTACTGTGCAAGAGAAGCCTAC
GGCCACTACTTTGACTACTGGGGCCAAGGCACCACTCTCACAGTCTCCTCA
G
Predicted Protein Sequence (complementarity determining regions (CDRs)
are in bold)
(SEQ ID NO: 29)
QVQLQQPGAELVRPGVSVKLSCKTSDYTFTSYWLHWFKQRPEQGLERIGEID
PSNGDADYSEKFKSKATLTVDKSSTTAYMQLASLTSEDSAVYYCAREAYGH
YFDYWGQGTTLTVSS
HC CDR1:
(SEQ ID NO: 30)
DYTFTSYW
HC CDR2:
(SEQ ID NO: 31)
IDPSNGDA
HC CDR3:
(SEQ ID NO: 32)
AREAYGHYFDY
Light Chain:
DNA Sequence
(SEQ ID NO: 33)
GATGTTGTGATGACCCAAACTCCACTCTCCCTGCCTGTCAGTCTTGGAGAT
CAAGCCTCCATCTCCTGCAGATCCAGTCAGAGCCTTGAACACAGTAATGG
AAACACCTATTTACATTGGTACCTGCAGAAGCCAGGCCAGTCTCCAAAGC
TCCTGATCTACAAACTTTCCAACCGATTTTCTGGGGTCCCAGACAGGTTCA
GTGGCAGTGGATCAGGGACAGATTTCACACTCAAGATCAGCAGAGTGGA
GGCTGAGGATCTGGGAGTTTATTTCTGCTCTCAAAGTACACATGTTCCGTA
CACGTTCGGAGGGGGGACCAAGCTGGAAATAAAAC
Predicted Protein Sequence (complementarity determining regions (CDRs)
are in bold)
(SEQ ID NO: 34)
DVVMTQTPLSLPVSLGDQASISCRSSQSLEHSNGNTYLHWYLQKPGQSPKLLI
YKLSNRFSGVPDRFSGSGSGTDFTLKISRVEAEDLGVYFCSQSTHVPYTFGGG
TKLEIK
LC CDR1:
(SEQ ID NO: 35)
QSLEHSNGNTY
LC CDR2:
(SEQ ID NO: 36)
KLS
LC CDR3:
(SEQ ID NO: 37)
SQSTHVPYT

### Antibodies

Antibodies of the present disclosure can include any type of antibodies such as unmodified (native) monoclonal antibodies, non-human animal antibodies, humanized antibodies, chimeric antibodies, human antibodies, minibodies, bi-specific antibodies (e.g., that bind oAβ and a receptor involved in RMT, such as the transferrin receptor (TfR), insulin receptor (IR), or the low-density lipoprotein receptor-related protein-1 (LRP-1)), amino acid sequence-modified antibodies, modified antibodies conjugated to other molecules (for example, polymers such as polyethylene glycol), and sugar chain-modified antibodies, as well as antigen-binding fragments thereof.

The antibodies used in the present methods and compositions are preferably isolated or purified. The terms “isolated” and “purified” used for substances described herein indicate that the substances do not substantially include at least one other substance that may be contained in the natural source. Therefore, “isolated antibodies” and “purified antibodies” refer to antibodies that do not substantially include cell materials such as hydrocarbons, lipids, or other contaminant proteins from the cell or tissue source from which the antibodies (proteins) are derived. When the antibodies are chemically synthesized, the terms refer to antibodies that do not substantially include chemical precursor substances or other chemical substances. In a preferred embodiment, the antibodies provided herein are isolated or purified.

A “monoclonal antibody” as used herein is intended to refer to one of a preparation of antibody molecules containing antibodies which share a common heavy chain and common light chain amino acid sequence, in contrast with an antibody from a “polyclonal” antibody preparation which contains a mixture of different antibodies. Monoclonal antibodies can be generated by several novel technologies like phage, bacteria, yeast or ribosomal display, as well as classical methods exemplified by hybridoma-derived antibodies (e.g., an antibody secreted by a hybridoma prepared by hybridoma technology, such as the standard Kohler and Milstein hybridoma methodology ((1975) Nature 256:495-497). Thus, a non-hybridoma-derived antibody is still referred to as a monoclonal antibody although it may have been derived by non-classical methodologies. In addition to the above-mentioned specificity, monoclonal antibodies have the advantage that they can be synthesized from a hybridoma culture that is not contaminated with other immunoglobulins. Therefore, “monoclonal” indicates the characteristics of antibodies that can be obtained from a substantially homogeneous antibody population. This term does not indicate the requirement for any specific method for antibody production.

An “isolated antibody”, as used herein, is intended to refer to an antibody that is substantially free of other antibodies having different antigenic specificities (e.g., an isolated antibody that specifically binds to a globulomer is substantially free of antibodies that specifically bind antigens other than a globulomer). An isolated antibody that specifically binds a globulomer may, however, have cross-reactivity to other antigens. Moreover, an isolated antibody can be substantially free of other cellular material and/or chemicals.

The term “antigen-binding fragment” or “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1969) Nature 341:544-546), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. j., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

Still further, an antibody or antigen-binding fragment thereof can be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

The term “chimeric antibody” refers to antibodies that comprise heavy and light chain variable region sequences from one species and constant region sequences from another species, such as antibodies having murine heavy and light chain variable regions linked to human constant regions.

The term “CDR-grafted antibody” refers to antibodies that comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies that comprise heavy and light chain variable region sequences from a nonhuman species (e.g., a mouse) but in which at least a portion of the VH and/or VL sequence has been altered to be more “human-like”, i.e., more similar to human germline variable sequences. One type of humanized antibody is a CDR-grafted antibody in which human CDR sequences are introduced into nonhuman VH and VL sequences to replace the corresponding nonhuman CDR sequences. In particular, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′) 2, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In other embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG 1, IgG2, IgG3 and IgG4. The humanized antibody may comprise sequences from more than one class or isotype, and particular constant domains can be selected to optimize desired effector functions using techniques well-known in the art.

The framework and CDR regions of a humanized antibody need not correspond precisely to the parental sequences, e.g., the donor antibody CDR or the consensus framework can be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. Further, as used herein, the term “consensus immunoglobulin sequence” refers to the sequence formed from the most frequently occurring amino acids (or nucleotides) in a family of related immunoglobulin sequences (see e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. (See also Kabat et al. Sequences of Proteins of Immunological Interest, Fifth Edition, U.S. Department of Health and Human Services, NIH Publication No. 91-3242, 1991). The human antibodies provided herein, however, may include amino acid residues not encoded by human germline immunoglobulin sequences (e.g., mutations introduced by random or site-specific mutagenesis in vitro or by somatic mutation in vivo). (See also Harlow and Lane, Antibodies: A Laboratory Manual, New York: Cold Spring Harbor Press, 1990).

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Recombinant human antibodies provided herein have variable regions, and may also include constant regions, derived from human germline immunoglobulin sequences. (See Kabat et al. (1991) supra.) In certain embodiments, however, such recombinant human antibodies are subjected to in vitro mutagenesis (or, when an animal transgenic for human Ig sequences is used, in vivo somatic mutagenesis), and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis or backmutation or both.

The term “backmutation” refers to a process in which some or all of the somatically mutated amino acids of a human antibody are replaced with the corresponding germline residues from a homologous germline antibody sequence. The heavy and light chain sequences of a human antibody described herein are aligned separately with the germline sequences in the VBASE database to identify the sequences with the highest homology. VBASE is a comprehensive directory of all human germline variable region sequences compiled from published sequences, including current releases of GenBank and EMBL data libraries. The database has been developed at the MRC Centre for Protein Engineering (Cambridge, UK) as a depository of the sequenced human antibody genes (website: mrc-cpe.cam.ac.uk/vbase-intro.php?menu=901). Differences in the antibodies described herein are returned to the germline sequence by mutating defined nucleotide positions encoding such different amino acids. The role of each amino acid thus identified as a candidate for backmutation should be investigated for a direct or indirect role in antigen binding, and any amino acid found after mutation to affect any desirable characteristic of the human antibody should not be included in the final human antibody. To minimize the number of amino acids subject to backmutation, those amino acid positions found to be different from the closest germline sequence, but identical to the corresponding amino acid in a second germline sequence, can remain, provided that the second germline sequence is identical and co-linear to the sequence of the human antibody for at least 10, preferably 12, amino acids on both sides of the amino acid in question. Backmutation may occur at any stage of antibody optimization.

A “labeled binding protein” is a protein wherein an antibody or antibody portion is derivatized or linked to another functional molecule (e.g., another peptide or protein). For example, a labeled binding protein can be derived by functionally linking an antibody or antibody portion (by chemical coupling, genetic fusion, noncovalent association or otherwise) to one or more other molecular entities, such as another antibody (e.g., a bispecific antibody or a diabody), detectable agent, a cytotoxic agent, a pharmaceutical agent, and/or a protein or peptide that can mediate associate of the antibody or antibody portion with another molecule (such as a streptavidin core region or a polyhistidine tag).

As used herein, a “glycosylated binding protein” comprises a protein wherein the antibody or antigen-binding fragment thereof comprises one or more carbohydrate residues. Nascent in vivo protein production may undergo further processing, known as post-translational modification. In particular, sugar (glycosyl) residues can be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R, Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993) 30:1361-1367), or result in increased affinity for the antigen (Wallick, S. C., et al., Exp. Med. (1988) 168:1099-1109; Wright, A., et al., EMBO J. (1991) 10:2717 2723). Further, glycosylation site mutants can be made in which the O- or N-linked glycosylation site of the binding protein has been mutated. One skilled in the art can generate such mutants using standard well-known technologies. Glycosylation site mutants that retain the biological activity but have increased or decreased binding activity are also contemplated.

Further, the glycosylation of the antibody or antigen-binding fragment can be modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such a glycosylation may increase the affinity of the antibody for antigen. Such an approach is described in further detail in International Application Publication No. WO 03/016466A2, and U.S. Pat. Nos. 5,714,350 and 6,350,861, each of which is incorporated herein by reference in its entirety.

Additionally or alternatively, a modified antibody can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. (See, for example, Shields, R. L. et al. (2002) J. Biol. Chem. 277:26733-26740; Umana et al. (1999) Nat. Biotech. 17:176-1, as well as, European Patent No: EP 1,176,195; International Application Publication Number WO 03/035835 and WO 99/5434280, each of which is incorporated herein by reference in its entirety.)

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. Preferably the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway can be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that host of a cell can be achieved by genetically modifying the host cell to express heterologous glycosylation enzymes. Using techniques known in the art a practitioner may generate antibodies or antigen-binding fragments thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. Patent Application Publication Nos. 20040018590 and 20020137134 and International Application Publication No. WO 05/100584 A2).

Further, it will be appreciated by one skilled in the art that a protein of interest can be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. Preferably, the protein having a particularly selected novel glycosylation pattern exhibits improved or altered biological properties.

The term “activity” includes activities such as the binding specificity/affinity of an antibody for an antigen.

For purposes of the present disclosure, a “fragment” of a sequence is defined as a contiguous sequence of approximately at least 6, preferably at least about 8, more preferably at least about 10 nucleotides, and even more preferably at least about 15 nucleotides corresponding to a region of the specified nucleotide sequence.

The term “identity” refers to the relatedness of two sequences on a nucleotide-by-nucleotide or amino acid-by-amino acid basis over a particular comparison window or segment. Thus, identity is defined as the degree of sameness, correspondence or equivalence between the same strands (either sense or antisense) of two DNA segments (or two amino acid sequences). “Percentage of sequence identity” is calculated by comparing two optimally aligned sequences over a particular region, determining the number of positions at which the identical base or amino acid occurs in both sequences in order to yield the number of matched positions, dividing the number of such positions by the total number of positions in the segment being compared and multiplying the result by 100. Optimal alignment of sequences can be conducted by the algorithm of Smith & Waterman, Appl. Math. 2:482 (1981), by the algorithm of Needleman & Wunsch, J. Mol. Biol. 48:443 (1970), by the method of Pearson & Lipman, Proc. Natl. Acad. Sci. (USA) 85:2444 (1988) and by computer programs which implement the relevant algorithms (e.g., Clustal Macaw Pileup (Higgins et al., CABIOS. 5L151-153 (1989)), FASTDB (Intelligenetics), BLAST (National Center for Biomedical Information; Altschul et al., Nucleic Acids Research 25:3389-3402 (1997)), PILEUP (Genetics Computer Group, Madison, Wis.) or GAP, BESTFIT, FASTA and TFASTA (Wisconsin Genetics Software Package Release 7.0, Genetics Computer Group, Madison, Wis.). (See U.S. Pat. No. 5,912,120.)

For purposes of the present disclosure, “complementarity” is defined as the degree of relatedness between two DNA segments. It is determined by measuring the ability of the sense strand of one DNA segment to hybridize with the anti-sense strand of the other DNA segment, under appropriate conditions, to form a double helix. A “complement” is defined as a sequence that pairs to a given sequence based upon the canonic base-pairing rules. For example, a sequence A-G-T in one nucleotide strand is “complementary” to T-C-A in the other strand.

“Similarity” between two amino acid sequences is defined as the presence of a series of identical as well as conserved amino acid residues in both sequences. The higher the degree of similarity between two amino acid sequences, the higher the correspondence, sameness or equivalence of the two sequences. (“Identity between two amino acid sequences is defined as the presence of a series of exactly alike or invariant amino acid residues in both sequences.) The definitions of “complementarity”, “identity” and “similarity” are well known to those of ordinary skill in the art.

“Encoded by” refers to a nucleic acid sequence which codes for a polypeptide sequence, wherein the polypeptide sequence or a portion thereof contains an amino acid sequence of at least 3 amino acids, more preferably at least 8 amino acids, and even more preferably at least 15 amino acids from a polypeptide encoded by the nucleic acid sequence.

Additionally, a nucleic acid molecule is “hybridizable” to another nucleic acid molecule when a single-stranded form of the nucleic acid molecule can anneal to the other nucleic acid molecule under the appropriate conditions of temperature and ionic strength (see Sambrook et al., “Molecular Cloning: A Laboratory Manual, Second Edition (1989), Cold Spring Harbor Laboratory Press, Cold Spring Harbor, N.Y.)). The conditions of temperature and ionic strength determine the “stringency” of the hybridization.

The term “hybridization” as used herein is generally used to mean hybridization of nucleic acids at appropriate conditions of stringency as would be readily evident to those skilled in the art depending upon the nature of the probe sequence and target sequences. Conditions of hybridization and washing are well known in the art, and the adjustment of conditions depending upon the desired stringency by varying incubation time, temperature and/or ionic strength of the solution are readily accomplished. See, for example, Sambrook, J. et al., Molecular Cloning: A Laboratory Manual, 2nd edition, Cold spring harbor Press, Cold Spring harbor, N.Y., 1989, as noted above and incorporated herein by reference. (See also Short Protocols in Molecular Biology, ed. Ausubel et al. and Tijssen, Techniques in Biochemistry and Molecular Biology-Hybridization with Nucleic Acid Probes, “Overview of principles of hybridization and the strategy of nucleic acid assays” (1993), both incorporated herein by reference.) Specifically, the choice of conditions is dictated by the length of the sequences being hybridized, in particular, the length of the probe sequence, the relative G-C content of the nucleic acids and the amount of mismatches to be permitted. Low stringency conditions are preferred when partial hybridization between strands that have lesser degrees of complementarity is desired. When perfect or near perfect complementarity is desired, high stringency conditions are preferred. For typical high stringency conditions, the hybridization solution contains 6×S.S.C., 0.01 M EDTA, 1×Denhardt's solution and 0.5% SDS. Hybridization is carried out at about 68 degrees Celsius for about 3 to 4 hours for fragments of cloned DNA and for about 12 to about 16 hours for total eukaryotic DNA. For moderate stringencies, one may utilize filter pre-hybridizing and hybridizing with a solution of 3×sodium chloride, sodium citrate (SSC), 50% formamide (0.1 M of this buffer at pH 7.5) and 5×Denhardts solution. One may then pre-hybridize at 37 degrees Celsius for 4 hours, followed by hybridization at 37 degrees Celsius with an amount of labeled probe equal to 3,000,000 cpm total for 16 hours, followed by a wash in 2×SSC and 0.1% SDS solution, a wash of 4 times for 1 minute each at room temperature and 4 times at 60 degrees Celsius for 30 minutes each. Subsequent to drying, one exposes to film. For lower stringencies, the temperature of hybridization is reduced to about 12 degrees Celsius below the melting temperature (Tm) of the duplex. The Tm is known to be a function of the G-C content and duplex length as well as the ionic strength of the solution.

“Hybridization” requires that two nucleic acids contain complementary sequences. However, depending on the stringency of the hybridization, mismatches between bases may occur. As noted above, the appropriate stringency for hybridizing nucleic acids depends on the length of the nucleic acids and the degree of complementation. Such variables are well known in the art. More specifically, the greater the degree of similarity or homology between two nucleotide sequences, the greater the value of Tm for hybrids of nucleic acids having those sequences. For hybrids of greater than 100 nucleotides in length, equations for calculating Tm have been derived (see Sambrook et al., supra). For hybridization with shorter nucleic acids, the position of mismatches becomes more important, and the length of the oligonucleotide determines its specificity (see Sambrook et al., supra).

As used herein, an “isolated nucleic acid fragment or sequence” is a polymer of RNA or DNA that is single- or double-stranded, optionally containing synthetic, non-natural or altered nucleotide bases. An isolated nucleic acid fragment in the form of a polymer of DNA can be comprised of one or more segments of cDNA, genomic DNA or synthetic DNA. (A “fragment” of a specified polynucleotide refers to a polynucleotide sequence which comprises a contiguous sequence of approximately at least about 6 nucleotides, preferably at least about 8 nucleotides, more preferably at least about 10 nucleotides, and even more preferably at least about 15 nucleotides, and most preferable at least about 25 nucleotides identical or complementary to a region of the specified nucleotide sequence.) Nucleotides (usually found in their 5′ -monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “fragment or subfragment that is functionally equivalent” and “functionally equivalent fragment or subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric constructs to produce the desired phenotype in a transformed plant. Chimeric constructs can be designed for use in co-suppression or antisense by linking a nucleic acid fragment or subfragment thereof, whether or not it encodes an active enzyme, in the appropriate orientation, relative to a plant promoter sequence.

The terms “homology”, “homologous”, “substantially similar” and “corresponding substantially” are used interchangeably herein. They refer to nucleic acid fragments wherein changes in one or more nucleotide bases does not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

“Gene” refers to a nucleic acid fragment that expresses a specific protein, including regulatory sequences preceding (5′ non-coding sequences) and following (3′ non-coding sequences) the coding sequence.

“Native gene” refers to a gene as found in nature with its own regulatory sequences. In contrast, “chimeric construct” refers to a combination of nucleic acid fragments that are not normally found together in nature. Accordingly, a chimeric construct may comprise regulatory sequences and coding sequences that are derived from different sources, or regulatory sequences and coding sequences derived from the same source, but arranged in a manner different than that normally found in nature. (The term “isolated” means that the sequence is removed from its natural environment.)

A “foreign” gene refers to a gene not normally found in the host organism, but that is introduced into the host organism by gene transfer. Foreign genes can comprise native genes inserted into a non-native organism, or chimeric constructs. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” or “regulatory gene sequence” refers to a DNA sequence capable of controlling the expression of a coding sequence or functional RNA. The sequence consists of proximal and more distal upstream elements, the latter elements often referred to as enhancers. Accordingly, an “enhancer” is a DNA sequence which can stimulate promoter or regulatory gene sequence activity and can be an innate element of the promoter or a heterologous element inserted to enhance the level or tissue-specificity of a promoter. Promoter sequences can also be located within the transcribed portions of genes, and/or downstream of the transcribed sequences. Promoters can be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most host cell types at most times are commonly referred to as “constitutive promoters”. New promoters of various types useful in plant cells are constantly being discovered; numerous examples can be found in the compilation by Okamuro and Goldberg, Biochemistry of Plants 15:1-82 (1989). It is further recognized that since in most cases the exact boundaries of regulatory sequences have not been completely defined, DNA fragments of some variation may have identical promoter activity.

An “intron” is an intervening sequence in a gene that does not encode a portion of the protein sequence. Thus, such sequences are transcribed into RNA but are then excised and are not translated. The term is also used for the excised RNA sequences. An “exon” is a portion of the gene sequence that is transcribed and is found in the mature messenger RNA derived from the gene, but is not necessarily a part of the sequence that encodes the final gene product.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell 1:671-680 (1989).

“RNA transcript” refers to the product resulting from RNA polymerase-catalyzed transcription of a DNA sequence. When the RNA transcript is a perfect complementary copy of the DNA sequence, it is referred to as the primary transcript or it can be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target gene (U.S. Pat. No. 5,107,065). The complementarity of an antisense RNA can be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “endogenous RNA” refers to any RNA which is encoded by any nucleic acid sequence present in the genome of the host prior to transformation with the recombinant construct provided herein, whether naturally-occurring or non-naturally occurring, i.e., introduced by recombinant means, mutagenesis, etc.

The term “non-naturally occurring” means artificial, not consistent with what is normally found in nature.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

The term “expression”, as used herein, refers to the production of a functional end-product. Expression of a gene involves transcription of the gene and translation of the mRNA into a precursor or mature protein. “Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020).

“Mature” protein refers to a post-translationally processed polypeptide; i.e., one from which any pre- or pro-peptides present in the primary translation product have been removed. “Precursor” protein refers to the primary product of translation of mRNA; i.e., with pre- and pro-peptides still present. Pre- and pro-peptides can be but are not limited to intracellular localization signals.

“Stable transformation” refers to the transfer of a nucleic acid fragment into a genome of a host organism, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The term “transformation” as used herein refers to both stable transformation and transient transformation.

Standard recombinant DNA and molecular cloning techniques used herein are well known in the art and are described more fully in Sambrook, J., Fritsch, E. F. and Maniatis, T., Molecular Cloning: A Laboratory Manual; Cold Spring Harbor Laboratory Press: Cold Spring Harbor, 1989 (hereinafter “Sambrook”).

The term “recombinant” refers to an artificial combination of two otherwise separated segments of sequence, e.g., by chemical synthesis or by the manipulation of isolated segments of nucleic acids by genetic engineering techniques.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

Polymerase chain reaction (“PCR”) is a powerful technique used to amplify DNA millions of fold, by repeated replication of a template, in a short period of time. (Mullis et al., Cold Spring Harbor Symp. Quant. Biol. 51:263-273 (1986); Erlich et al., European Patent Application No. 50,424; European Patent Application No. 84,796; European Patent Application No. 258,017; European Patent Application No. 237,362; Mullis, European Patent Application No. 201,184; Mullis et al., U.S. Pat. No. 4,683,202; Erlich, U.S. Pat. No. 4,582,788; and Saiki et al., U.S. Pat. No. 4,683,194). The process utilizes sets of specific in vitro synthesized oligonucleotides to prime DNA synthesis. The design of the primers is dependent upon the sequences of DNA that are to be analyzed. The technique is carried out through many cycles (usually 20- 50) of melting the template at high temperature, allowing the primers to anneal to complementary sequences within the template and then replicating the template with DNA polymerase.

The products of PCR reactions are analyzed by separation in agarose gels followed by ethidium bromide staining and visualization with UV transillumination. Alternatively, radioactive dNTPs can be added to the PCR in order to incorporate label into the products. In this case the products of PCR are visualized by exposure of the gel to x-ray film. The added advantage of radiolabeling PCR products is that the levels of individual amplification products can be quantitated.

The terms “recombinant construct”, “expression construct” and “recombinant expression construct” are used interchangeably herein. These terms refer to a functional unit of genetic material that can be inserted into the genome of a cell using standard methodology well known to one skilled in the art. Such construct can be itself or can be used in conjunction with a vector. If a vector is used then the choice of vector is dependent upon the method that will be used to transform host plants as is well known to those skilled in the art. For example, a plasmid can be used. The skilled artisan is well aware of the genetic elements that must be present on the vector in order to successfully transform, select and propagate host cells comprising any of the isolated nucleic acid fragments described herein. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening can be accomplished by Southern analysis of DNA, Northern analysis of mRNA expression, Western analysis of protein expression, or phenotypic analysis.

The terms “isolated” and “purified” used for substances (antibodies and such) provided herein indicate that the substances do not substantially include at least one other substance that can be contained in the natural source. Therefore, “isolated antibodies” and “purified antibodies” refer to antibodies that do not substantially include cell materials such as hydrocarbons, lipids, or other contaminant proteins from the cell or tissue source from which the antibodies (proteins) are derived. When the antibodies are chemically synthesized, the terms refer to antibodies that do not substantially include chemical precursor substances or other chemical substances. In a preferred embodiment, the antibodies of the present invention are isolated or purified.

### Methods of Making Antibodies

The invention also provides a method for making monoclonal antibodies from non-human, non-mouse animals by immunizing non-human transgenic animals that comprise human immunoglobulin loci with a peptide immunogen as described herein. One may produce such animals using methods known in the art. In a preferred embodiment, the non-human animals can be rats, sheep, pigs, goats, cattle or horses. Antibody-producing immortalized hybridomas can be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using an antigen (for example, a globulomer) or a portion thereof, or a cell expressing the antigen of interest. In a preferred embodiment, the initial screening is performed using an enzyme-linked immunoassay (ELISA) or a radioimmunoassay (RIA), preferably an ELISA. An example of ELISA screening is provided in International Application Publication No. WO 00/37504, herein incorporated by reference.

The antibody-producing hybridomas are selected, cloned and further screened for desirable characteristics, including robust hybridoma growth, high antibody production and desirable antibody characteristics, as discussed further below. Hybridomas can be cultured and expanded in vivo in syngeneic animals, in animals that lack an immune system, e.g., nude mice, or in cell culture in vitro. Methods of selecting, cloning and expanding hybridomas are well known to those of ordinary skill in the art. Preferably, the immunized animal is a non-human animal that expresses human immunoglobulin genes and the splenic B cells are fused to a myeloma derived from the same species as the non-human animal.

In one aspect, the invention provides hybridomas that produce monoclonal antibodies to be used in the treatment, diagnosis and prevention of Alzheimer's Disease. In a preferred embodiment, the hybridomas are mouse hybridomas. In another preferred embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an antibody against a globulomer.

Recombinant antibodies can be generated from single, isolated lymphocytes using a procedure referred to in the art as the selected lymphocyte antibody method (SLAM), as described in U.S. Pat. No. 5,627,052, International Application Publication No. WO 92/02551 and Babcock, J. S. et al. (1996) Proc. Natl. Acad. Sci. USA 93: 7843-7848. In this method, single cells secreting antibodies of interest (e.g., lymphocytes derived from the immunized animal) are screened using an antigen-specific hemolytic plaque assay, wherein the antigen (e.g., globulomer), or a fragment thereof, is coupled to sheep red blood cells using a linker, such as biotin, and used to identify single cells that secrete antibodies with specificity for the antigen. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to IL-18. The amplified immunoglobulin sequences further can be manipulated in vitro, such as by in vitro affinity maturation methods such as those described in International Application Publication No. WO 97/29131 and International Application Publication. No. WO 00/56772.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphory, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “immunize” refers herein to the process of presenting an antigen to an immune repertoire whether that repertoire exists in a natural genetically unaltered organism, or a transgenic organism modified to display an artificial human immune repertoire. Similarly, an “immunogenic preparation” is a formulation of antigen that contains adjuvants or other additives that would enhance the immunogenicity of the antigen. An example of this would be co-injection of a purified peptide or peptide immunogen with Freund's complete adjuvant into a mouse; in some embodiments, the adjuvant is covalently linked to the peptide. “Hyperimmunization”, as defined herein, is the act of serial, multiple presentations of an antigen in an immunogenic preparation to a host animal with the intention of developing a strong immune response.

It should also be noted that the subject invention not only includes the full length antibodies described above but also portions or fragments thereof, for example, the Fab portion thereof. Additionally, the subject invention encompasses any antibody having the same properties of the present antibodies in terms of, for example, binding specificity, structure, etc.

“Antibodies” refers to glycoproteins that have the same structural characteristics. Antibodies show binding specificity towards specific antigens. Herein, “antigens” refers to proteins that have the ability to bind to the corresponding antibodies, and induce antigen-antibody reactions in vivo.

A proteins, which are the major constituents of amyloids, are peptides consisting of 40 to 42 amino acids, and are known to be produced from precursor proteins called amyloid precursor proteins (APPs) by the action of proteases. Besides amyloid fibrils collected in ultracentrifuged sediment fractions, the amyloid molecules produced from APPs include oligomeric non-fibrous assemblies in addition to soluble monomers. “Aβ oligomers” refers to non-fibrous assemblies, which can include, for example, Aβ40 (Aβ1-40) oligomers and Aβ42 (Aβ1-42) oligomers, or combinations thereof. For example, “Aβ42 oligomers” as described herein are molecules showing a molecular weight of 45 to 160 kDa in SDS-PAGE, and 22.5 to 1,035 kDa in Blue Native PAGE. Using molecular sieves, the molecules are collected mainly in the >100 kDa retention solution. When observed under an atomic force microscope, the molecules show mixed morphologies of granular, bead-shaped, and ring-shaped molecules having a height of 1.5 to 3.1 nm. By the gel filtration method, the molecules can be eluted in the void volume fraction 8 with a molecular weight of 680 kDa or more, and in fraction 15 with a molecular weight of 17 to 44 kDa.

There is no limitation on the origin and form of the antibodies as described herein as long as they bind to Aβ oligomers but not to Aβ monomers.

Monoclonal antibodies can be produced by using known techniques. For example, they can be produced by the hybridoma method first described by Kohler and Milstein (Nature 256: 495-7, 1975), or by the recombinant DNA method (Cabilly et al., Proc. Natl. Acad. Sci. USA 81:3273-7, 1984), but the methods are not limited thereto. For example, when using the hybridoma method, an Aβ oligomer (for example, the Aβ tetramer described in the Examples) is used as a sensitizing antigen, and immunization is carried out according to a conventional immunization method. The obtained immune cells are fused with known parent cells by a conventional cell fusion method, and monoclonal antibody-producing cells can be screened and isolated using a conventional screening method.

Monoclonal antibodies can be produced as follows. Balb-c mice are immunized with a peptide immunogen as described herein, optionally emulsified using complete Freund's adjuvant by injecting the antigen into their foot pad. Subsequently, booster immunizations are carried, e.g., out six times. Hybridomas are then produced from the inguinal lymph node, e.g., by fusion with Sp2/O-Ag14 cells using Polyethylene Glycol 1500.

The animals immunized with sensitizing antigens are not particularly limited, but are preferably selected considering the compatibility with parent cells used for cell fusion. Generally, rodents, lagomorphs, or primates are used. Rodents include, for example, mice, rats, and hamsters. Lagomorphs include, for example, rabbits. Primates include, for example, Catarrhini (old-World) monkeys such as Macaca fascicularis, Macaca mulatta, hamadryas, and chimpanzees.

Animals are immunized with sensitizing antigens according to known methods. For example, as a standard method, immunization is performed by intraperitoneal or subcutaneous injection of a sensitizing antigen into mammals.

An example of the parent cells fused with the aforementioned immunocytes is the Sp2/O-Ag14 cell, which will be described below in the Examples. However, various other known cell lines can be used.

Cell fusion between the aforementioned immunocyte and a myeloma cell can be carried out basically according to known methods including the method by Kohler and Milstein (Kohler and Milstein C., Methods Enzymol. (1981) 73, 3-46).

Hybridomas obtained in this Manner are selected by culturing them in a conventional selection culture medium such as a HAT culture medium, which contains hypoxanthine, aminopterin, and thymidine. Culturing in the above-mentioned HAT culture medium is generally continued for several days to several weeks for an adequate time for killing cells other than the desired hybridomas (non-fused cells). Next, a conventional limiting dilution method is performed for screening and singly-cloning of a hybridoma that produces the desired antibody.

Thereafter, the obtained hybridoma is transplanted into the abdominal cavity of a mouse, and ascitic fluid containing the desired monoclonal antibodies is extracted. For example, the antibodies can be purified from the ascitic fluid by conventional protein separation and/or purification methods such as a selected combination of column chromatography including, but not limited to, affinity chromatography, filtration, ultrafiltration, salt precipitation, dialysis, SDS polyacrylamide gel electrophoresis, and isoelectric focusing (Antibodies: A Laboratory manual, Harlow and David, Lane (edit.), Cold Spring Harbor Laboratory, 1988).

Protein A columns and Protein G columns can be used for affinity columns. Examples of the Protein A columns used include Hyper D, POROS, and Sepharose F.F. (Pharmacia).

Chromatography (excluding affinity chromatography) includes ion exchange chromatography, hydrophobic chromatography, gel filtration, reverse-phase chromatography, and adsorption chromatography (“Strategies for Protein Purification and Characterization: A Laboratory Course Manual”, Daniel R Marshak et al., Cold Spring Harbor Laboratory Press, 1996). When chromatography is carried out, liquid-phase chromatography methods such as HPLC and FPLC can be used.

Monoclonal antibody-producing hybridomas prepared in this manner can be subcultured in a conventional culture medium, and they can be stored for a long time in liquid nitrogen.

Any mammal can be immunized using an immunogen for antibody production. However, when preparing monoclonal antibodies by producing hybridomas, the compatibility with parent cells used in cell fusion for hybridoma production is preferably considered.

Generally, rodents, lagomorphs, or primates are used for the immunization. Rodents include, for example, mice, rats, and hamsters. Lagomorphs include, for example, rabbits. Primates include, for example, Catarrhini (old-world) monkeys such as Macaca fascicularis, Macaca mulatta, hamadryas, and chimpanzees.

The use of transgenic animals that have a human antibody gene repertoire is known in the art (Ishida et al., Cloning and Stem Cells 4: 91-102, 2002). As with other animals, to obtain human monoclonal antibodies, the transgenic animals are immunized, then antibody-producing cells are collected from the animals and fused with myeloma cells to produce hybridomas, and anti-protein human antibodies can be prepared from these hybridomas (see International Publication Nos. WO92/03918, WO94/02602, WO94/25585, WO96/33735, and WO96/34096).

Alternatively, lymphocytes immortalized with oncogenes can be used for monoclonal antibody production. For example, human lymphocytes infected with EB virus or such is immunized in vitro with immunogens. Next, the immunized lymphocytes are fused with human-derived myeloma cells (U266, etc.) capable of unlimited division, and thus hybridomas that produce the desired human antibodies are obtained (Japanese Patent Application Kokai Publication No. (JP-A) S63-17688 (unexamined, published Japanese patent application)).

Once monoclonal antibodies are obtained by any of the aforementioned methods, the antibodies may also be prepared using genetic engineering methods (see, for example, Borrebaeck C A K and Larrick J W, Therapeutic Monoclonal Antibodies, MacMillan Publishers, U K, 1990). For example, recombinant antibodies can be prepared by cloning DNAs that encode the desired antibodies from antigen-producing cells such as hybridomas or immunized lymphocytes that produce the antibodies, then inserting the cloned DNAs into appropriate vectors, and transecting the vectors into suitable host cells. Such recombinant antibodies are also included in the present invention.

### Methods, Compositions, and Kits for Diagnosis

Despite the mounting evidence that soluble oAβ plays a central role in early AD pathogenesis, there exists a major unmet need for a sensitive and specific method to detect and quantify oAβ in non-invasively, e.g., in human plasma. Monoclonal antibodies 1G5 and 71A1, which were generated by using a synthetic Aβ1-40 cyclic peptide as immunogen, have been extensively characterized and used to develop oligomer-specific immunoassays as described herein. As shown herein, 1) 1G5 and 71A1 recognize soluble oAβ from human brain, CSF and plasma without reactivity to Aβ monomers; 2) 71A1 neutralizes the synaptotoxicity of oAβ-rich AD brain extracts in electrophysiological assays; and 3) an ultra-sensitive immunoassay using 71A1 as capture paired with 3D6 as detector achieves a lower LLoQ of 0.6 pg/mL and reliably quantifies oAβ in human brain, CSF and plasma with low coefficients of variation (CVs). The availability of a sensitive and specific immunoassay that quantifies endogenous human oAβ in blood should enable future studies of the dynamic process of Aβ oligomerization and disassembly in brain and biofluids and establish correlations between oAβ and other known AD biomarkers including Aβ monomers [32-34], Tau fragments [35, 36] and pTau [37-43] in the blood. Moreover, these findings support the potential therapeutic benefit of neutralizing 71A1-immunoreactive oAβ. Together, these advances deepen our understanding of the pathobiological role of oAβ in AD and pave the way to monitoring this key pathogenic form of amyloid b-protein in Alzheimer's disease and age-related β-amyloidosis.

Since the detection of synthetic and natural Aβ oligomers modified the amyloid hypothesis some two decades ago [1, 2], there have been continuous efforts to generate better tools to visualize and distinguish oAβ from Aβ monomers and fibrils, the structure of which could overlap in part with smaller oligomeric species. Monoclonal antibodies have been developed in an attempt to recognize and even neutralize soluble oAβs, which are believed to be the more synaptotoxic forms of Aβ relative to insoluble Aβ plaques in the brain [3, 4, 14]. Among them, monoclonal antibody 1C22 [26] has been shown to be highly specific to oAβ in both the brain and CSF [30, 53]. We previously quantified 1C22-positive oligomers in CSF from 104 AD subjects participating in the ABBY and BLAZE phase 2 trials of the anti-Aβ antibody Crenezumab from Roche and found that oligomer Aβ levels measured by the 1C22/3D6 assay were significantly decreased in a high proportion of Crenezumab-treated patients, whereas no systematic change occurred in the placebo group [31]. These data encouraged us to engage further in the testing of novel antibodies to oAβ with potentially greater avidity and specificity. Through the design of a cyclized Aβ peptide immunogen to potentially mimic a dimeric conformation, positive clones 1G5 and 71A1 demonstrated high binding capability and specificity to the immunogen and to synthetic Aβ oligomers. In this study, we have validated these two novel antibodies with regard to their capacity to a) specifically recognize oAβ; b) quantify oAβ in human brain extracts, CSF and plasma; and c) neutralize the synaptotoxicity of diffusible Aβ oligomers derived from AD cerebral cortex.

We initially examined the ability of 1G5 and 71A1 to bind Aβ assemblies in AD brain soaking extracts, which have been validated to contain highly diffusible and synaptotoxic oAβ species [11]. We found that both 1G5 and 71A1 successfully pulled down Aβ as examined by IP-immunoblots probed with both mid-region and N-terminal (Asp-1) specific antibodies, and by IP-ELISA using ELISAs detecting Aβ x-40 and x-42. Interestingly, compared to our oligomer-specific benchmark antibody 1C22 [30], both 1G5 and 71A1 showed much lower binding capacity toward soluble Aβ species in the human brain soaking extracts. Similarly, when we compared these three antibodies by immunohistochemistry, 1G5 and 71A1 recognized plaques in unfixed cryostat sections, as confirmed through double labeling with a standard Aβ antibody D54D2. All three oligomer-preferring antibodies stained Aβ deposits well in native cryo-sections, but the staining intensities were substantially diminished by fixation of the sections with 4% PFA, suggesting that these antibodies recognize conformational epitopes sensitive to fixative crosslinking. 71A1 and 1G5 recognized a smaller portion of Aβ deposits in the brain than did 1C22. Nonetheless, 71A1 showed a consistent ability to protect against the inhibition of hippocampal LTP induced by AD brain soaking extracts, corroborating the previous finding that the small portion of brain Aβ (˜12%) obtained by simple diffusion out of cortical pieces over just 30 minutes confers much of the recoverable synaptotoxicity [11]. Moreover, oAβ affinity-purified with 71A1 from AD soaking extracts confers potent synaptotoxicity, and in turn, 71A1 can neutralize the toxicity of AD brain-derived soluble oAβ, demonstrating the attractive potentially therapeutic properties of this new antibody.

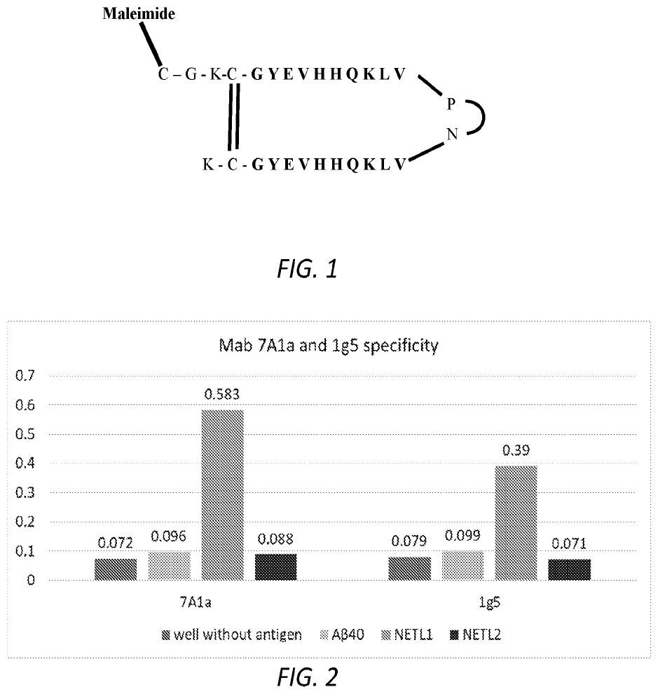

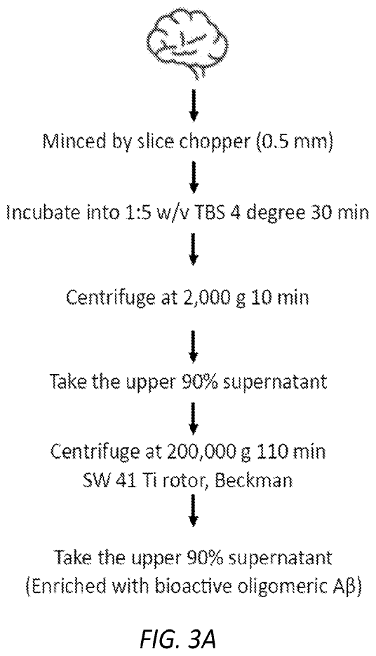

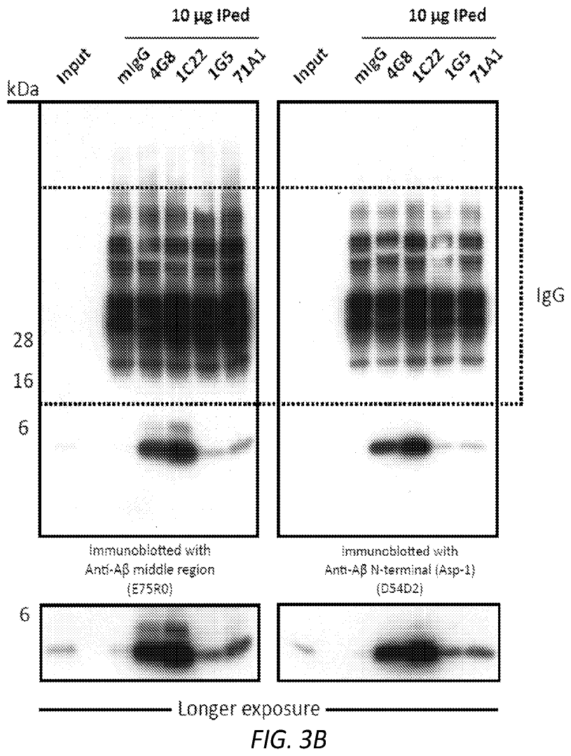

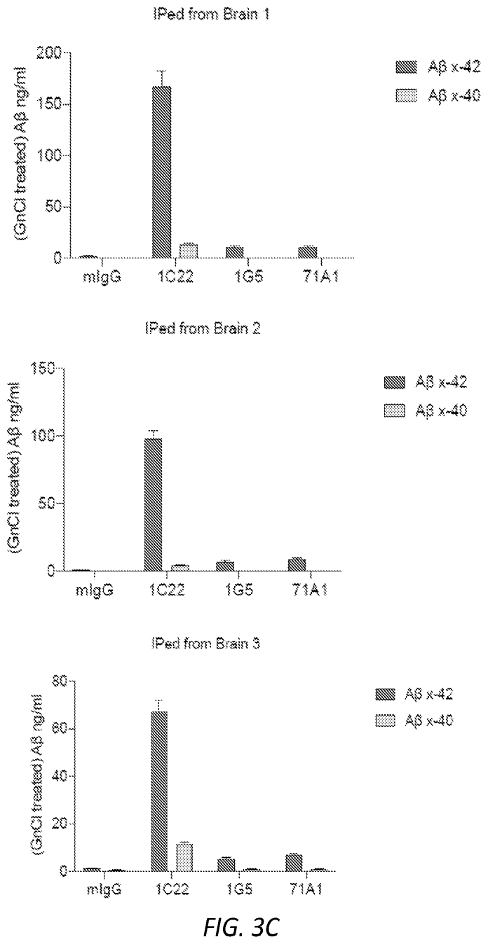

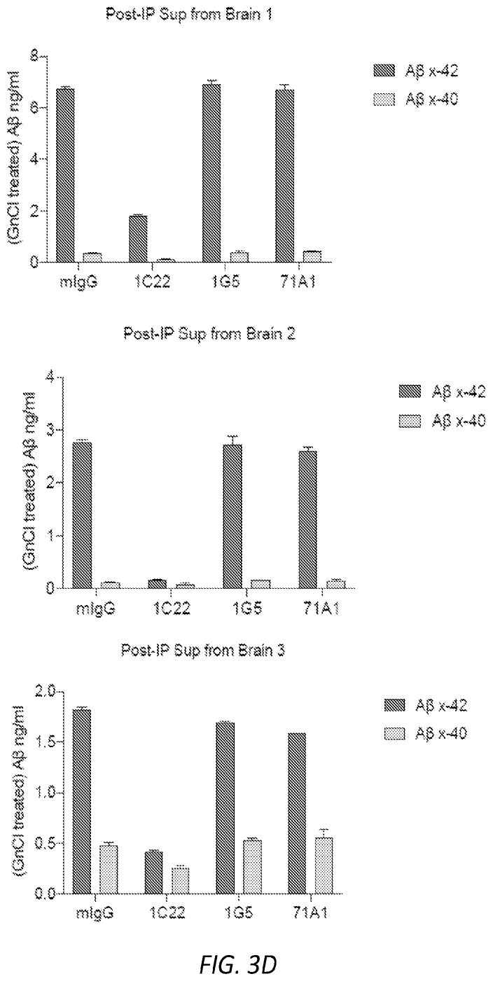

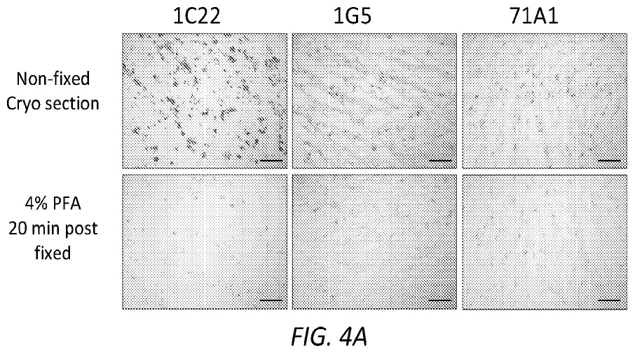

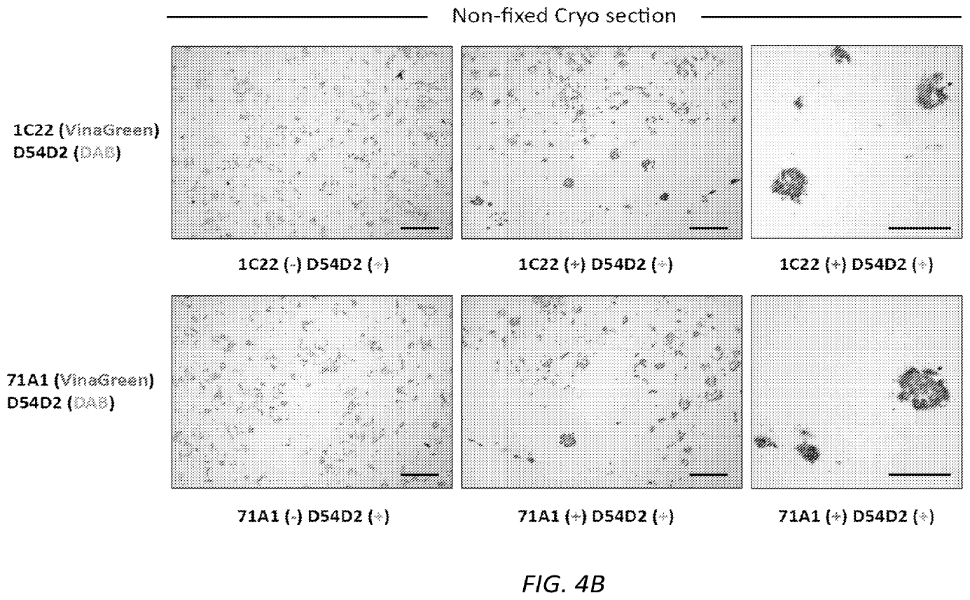

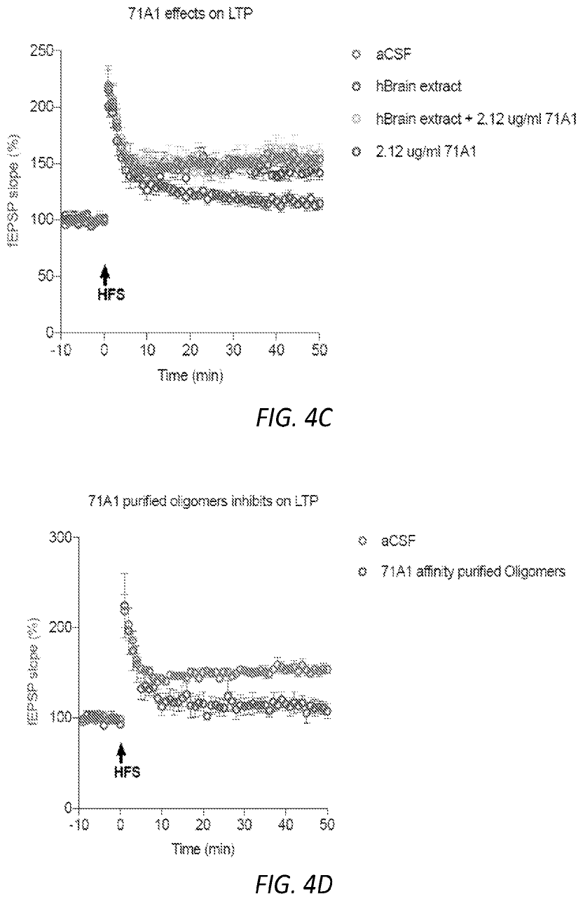

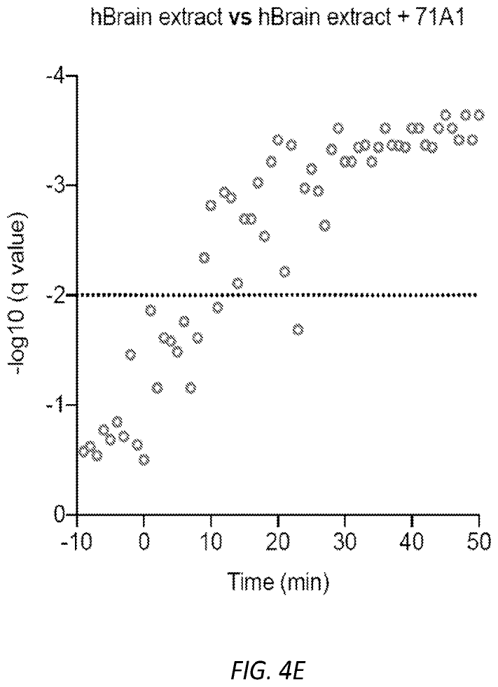

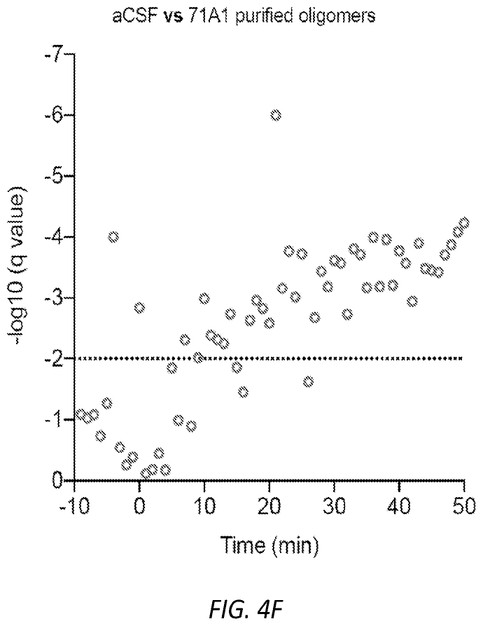

In contrast to these data on soluble Aβ from brain (FIG. 1C), our IP-ELISA showed that the rank order of Aβ binding capability in CSF was reversed: 71A1>1G5>>1C22 (FIG. 3A). This difference between the brain soaking extracts and CSF is striking and can be interpreted to suggest a difference in soluble oAβ assemblies between brain and CSF. Furthermore, all three of these oAβ-preferring antibodies immunoprecipitated Aβ40 and Aβ42 equally, suggesting oAβ in CSF contains diverse Aβ monomer variants. To more accurately quantify low levels of oAβ from biofluids such as plasma beyond immunoprecipitation, we developed an ultra-sensitive sandwich immunoassay on the Erenna SMCxPRO platform (Millipore) using 71A1 as capture antibody and 3D6 (to the Aβ Asp-1 N-terminus) as detector antibody, achieving a LLoQ of 0.6 pg/mL and high reproducibility as demonstrated by low intra-assay % CVs (<10%) and excellent (˜100%) recovery ratios (FIG. 4D). The specificity of the 71A1/3D6 immunoassay toward oAβ was demonstrated by 71A1's strong preference for synthetic ADDLs [1] and S26C Aβ1-40 dimers [45] over their monomeric components (FIG. 4).

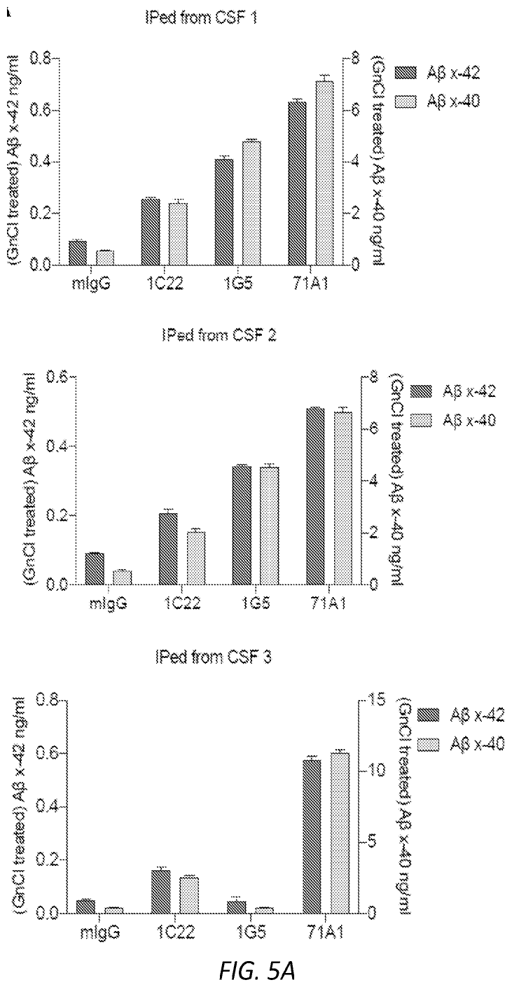

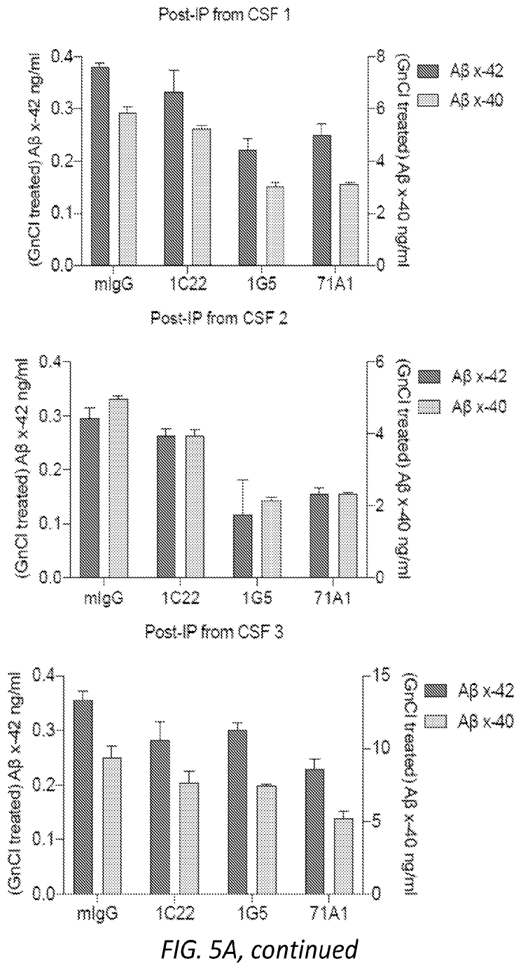

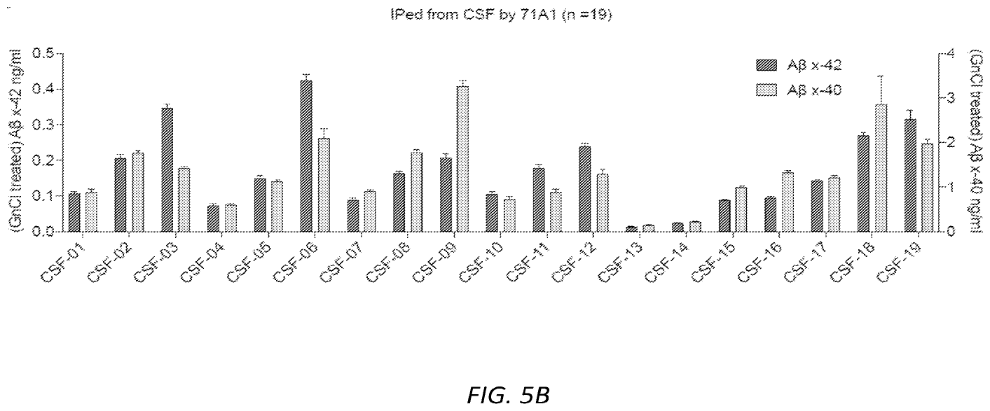

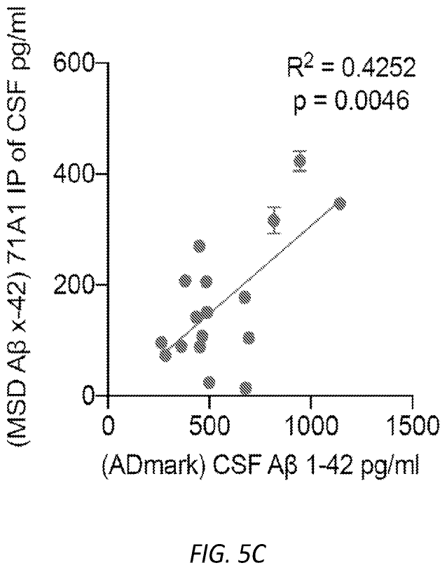

After thus validating the stability and relative oligomer specificity of the 71A1/3D6 immunoassay on oAβ prepared from synthetic Aβ peptides, we performed the assay on native SEC fractions of AD brain soaking extracts and observed that the assay recognized the high MW Aβ oligomers without recognizing Aβ monomers (FIG. 5C, D), similar to the oligomer-specific 1C22/3D6 assay previously reported [53]. After confirming the oligomer-specificity of the 71A1/3D6 assay, we proceeded to quantify oAβ levels in human CSFs and plasmas. Importantly, we found that the 71A1-immunoreactive oAβ levels in CSF (n=29) were significantly correlated with total tau and pT181 tau in the same samples, as measured by the CLIA-approved ADmark assay. However, we did not observe a correlation between 71A1/3D6 signals and Aβ 1-42 monomer levels.

We then proceeded to characterize the 71A1/3D6 immunoassay on human plasma, the major goal of this study. To demonstrate the validity of quantifying oAβ in plasma without the effects of plasma matrix interference, we investigated: 1) dilution recovery; 2) spike-and-recovery with 3 different natural sources of Aβ (human brain soluble homogenate, human brain soaking extract and CSF); 3) immunodepletion by 71A1, 1C22, and negative-control antibody 4-64 (raised against HIV glycoprotein 120); and 4) brain homogenate, CSF, and plasma treated with GnCl (a potent chaotropic salt that rapidly disassembles oAβ into monomers). The 71A1/3D6 immunoassay passed all these tests, showing 1) optimal recovery in both plasma dilution and spike-in experiments; 2) immunodepletable signals by 71A1 but not by 1C22 or control antibody; and 3) markedly reduced or abolished 71A1 signals after GnCl treatment of all 3 sample types. Finally, we conducted the 71A1/3D6 assay on a cohort of plasmas from 73 cognitively normal human subjects (demographic data in Table 1) and obtained an average (+/−SD) concentration of 71A1-positive oligomers in plasma of 43.34±29.09 pg/mL.

Our study provides several salient findings. First, we report the design and detailed characterization of new antibodies which recognize conformational epitopes in natural human oligomeric Aβ. Second, we show that 1G5 and 71A1 have higher binding capability to oAβ in biofluids than a reference oligomer-preferring antibody, 1C22. Third, 71A1 neutralizes the synaptic toxicity of highly diffusible oligomers in AD brain soaking extracts. And fourth, the novel 71A1/3D6 sandwich ELISA sensitively and reliably quantifies oAβ in human plasma. To our knowledge, this is the first report of quantifying oAβ in human plasma using an oligomer-selective conformational antibody. This assay will now become a unique tool to probe the biology of endogenous oAβ, including its structural properties, its dynamics in human plasma and CSF, and its functional cytotoxicity. We found three reports of oAβ plasma immunoassays [55-57], all of which involve using identical capture and detector antibodies, thus requiring at least two identical exposed epitopes on the surface for quantification. One such assay using 82E1 as both capture and detector antibody is commercially available from Immuno-Biological Laboratories (IBL). Those assays would recognize a broad range of Aβs from dimer to protofibril and even fibril, as long as the N-terminal epitopes are available and exposed. Also, all the reported assays lack detailed characterizations, such as dilution linearity, spike-in recovery and immunodepletion to test the assay specificity. The exeplary 71A1/3D6 assay described herein exhibits improved specificity by using a novel oligomer-specific antibody that does not detect monomers as capture and improved sensitivity via the SMCxPRO system.

Now that it has been established and technically validated, the new assay can be used by us and others to systematically examine large, well-defined cross-sectional and longitudinal human cohorts and correlate plasma oAβ levels with other established and emerging AD biomarkers, such as Aβ monomer assays that detect all six C-terminal variants from Aβ37 to Aβ 43 [32], the NT-1 tau assay to detect N-terminal tau fragments which correlate with multiple AD phenotypes [35, 36], and highly promising recent assays that detect tau phosphorylated at Thr181, Thr217 and Thr231 (see Introduction). Such detailed correlative analyses on multiple cohorts should reveal further insights into the involvement of oAβ dynamics in the early pathogenesis and course of AD, including to monitor the effects of emerging anti-amyloid treatments in CSF and plasma. The creation, analytical validation and initial application of this sensitive and specific assay for endogenous oAβ in human plasma provides a long-sought method to detect and follow the species that extensive evidence from many laboratories suggests is the key bioactive from of Aβ in AD and thus a major target for disease-modifying therapeutics.

Thus, provided herein are methods for assaying oAβ present in a sample comprising one or more biological fluids, e.g., as described herein, e.g., from a human subject, or an animal model, e.g., a humanized APP mouse, or a veterinary subject, e.g., a cat or a dog. The methods can be used for quantitatively assaying oAβ in the sample. The methods can be used on a fluid sample from a human patient, e.g., a sample comprising blood or plasma. The sample can be obtained using methods known in the art. Samples can include, e.g., tissue, whole blood, plasma, serum, urine, saliva, breath, exosome or exosome-like microvesicles, lymph, cerebrospinal fluid, or sputum.

In some embodiments, a sandwich immunoassay is used, in which capture antibodies bound to a surface (e.g., a surface such as the surface of a plate or a bead) and a labeled detection antibody. The methods can include capturing oAβ present in the sample with a first agent or probe (the capture antibody) that recognizes and binds to oAβ in the sample. Preferably, the capture antibody is a 71A1 or 1G5 antibody or antigen-binding fragment thereof as described herein. The capture antibody is preferably attached to a surface, e.g., the surface of a plate, such as a multiwell plate, the surface of a bead, e.g., beads, particles, microspheres, nanotubes, polymers, plates, disks, or dipsticks. Beads can include magnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, or TEFLON® beads. In some embodiments, the capture antibody is conjugated to a biotin moiety, and the surface is coated with streptavidin, and the antibody is attached to the surface via biotin-streptavidin binding.

The sample is contacted with a second, detection antibody that binds to the oAβ-capture antibody complexes. For example, the detection antibody can bind to oAβ but recognize a different epitope on said target analyte and doesn't compete for binding to oAβ with the capture (e.g., 71A1 or 1G5) antibody. Exemplary detection antibodies that bind to oAβ include 3D6 (see, e.g., Feinberg et al. Alzheimers Res Ther. 2014; 6:31) or 82E1; 71A1 or 1G5 could also be used as detectors in a sandwich assay. Alternatively, the capture antibody can comprise an affinity tag and the detection antibody can bind to the affinity tag. Examples of affinity tags include Hemagglutinin (HA), HIS tag, FLAG tag (DYKDDDDK, SEQ ID NO:38), Glutathione S Transferase (GST), green fluorescent protein (GFP), S-tag, Strep-tag, VSVG, V5, myelin basic protein (MBP), c-myc, and combinations thereof (see, e.g., Kimple et al., Curr Protoc Protein Sci. 2013; 73: Unit-9.9); detection antibodies that bind to tags are known and commercially available, e.g., from BioRad, ThermoFisher, and other sources. Preferably the detection antibody is labeled, e.g., with a radioisotope, a chemiluminescent substance, a fluorescent substance, a metal complex, a bioluminescent substance such as a luciferase, or a nucleic acid.

The methods further include detecting and optionally quantifying the concentration of oAβ in the sample, e.g., using a radio immunoassay method (RIA) when the detection antibody is labeled with a radioisotope, a chemiluminescent immunoassay method (CIA) when the detection antibody is labeled with a chemiluminescent substance, a fluorescent immunoassay method (FIA) when the detection antibody is labeled with a fluorescent substance, an electro-chemiluminescent immunoassay method (ECLIA) when the detection antibody is labeled with a metal complex, a bioluminescent immunoassay method (BLIA) when the detection antibody is labeled with a bioluminescent substance such as a luciferase, an immuno-PCR comprising amplifying a nucleic acid that labeled an antibody by PCR and detecting the nucleic acid, when the detection antibody is labeled with a nucleic acid, a turbidimetric immunoassay method (TAI) detecting turbidity occurred by forming an immunocomplex, a latex agglutination turbidimetric method (LA) detecting a latex aggregated by forming an immunocomplex, or an immunochromatography assay utilizing a reaction on a cellulose membrane. In some embodiments, the method is a fluorescence based ELISA platform such as SMCxPRO or Erenna (MilliporeSigma).

Detection can be facilitated by coupling the antibody, or antigen-binding fragment, variant, or derivative thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions; see, e.g., U.S. Pat. No. 4,741,900 for metal ions which can be conjugated to antibodies for use as diagnostics. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc. In some embodiments the fluorescent label is a bright, far-red-fluorescent dye with excitation ideally suited for the 594 nm or 633 nm laser lines. In some embodiments, the label is or comprises a fluorescent protein. A number of reporter proteins are known in the art, and include green fluorescent protein (GFP), variant of green fluorescent protein (GFP10), enhanced GFP (eGFP), TurboGFP, GFPS65T, TagGFP2, mUKGEmerald GFP, Superfolder GFP, GFPuv, destabilised EGFP (dEGFP), Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, destabilised ECFP (dECFP), CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSSmOrange, CyOFP1, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP657, TagRFP675, mKate2, HcRed, t-HcRed, HcRed-Tandem, mPlum, mNeptune, NirFP, Kindling, far red fluorescent protein, yellow fluorescent protein (YFP), eYFP, destabilised EYFP (dEYFP), TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKOK, mKO2, mGrapel, mGrape2, zsYellow, eqFP611, Sirius, Sandercyanin, shBFP-N158S/L173I, near infrared proteins, iFP1.4, iRFP713, iRFP670, iRFP682, iRFP702, iRFP720, iFP2.0, mIFP, TDsmURFP, miRFP670, Brilliant Violet (BV) 421, BV 605, BV 510, BV 711, BV786, PerCP, PerCP/Cy5.5, DsRed, DsRed2, mRFPl, pocilloporin, Renilla GFP, Monster GFP, paGFP, or a Phycobiliprotein, or a biologically active variant or fragment of any one thereof. Other proteins such as luciferase can also be used.

An antibody, or antigen-binding fragment, variant, or derivative thereof also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an antibody, or antigen-binding fragment, variant, or derivative thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2 (1978), 1-7); Voller et al., J. Clin. Pathol. 31 (1978), 507-520; Butler, Meth. Enzymol. 73 (1981), 482-523; Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibody, or antigen-binding fragment, variant, or derivative thereof, it is possible to detect the antibody through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

An antibody, or antigen-binding fragment, variant, or derivative thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to an antibody, or antigen-binding fragment, variant, or derivative thereof are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62 (1982), 119-158.

Included herein are methods for diagnosing a subject as having amyloidosis, e.g., CAA or Alzheimer's disease (AD). The methods include obtaining a sample from a subject, and evaluating the presence and/or level of oAβ in the sample, and comparing the presence and/or level with one or more references, e.g., a control reference that represents a normal level of oAβ, e.g., a level in an unaffected subject, and/or a disease reference that represents a level of the proteins associated with an amyloidosis (e.g., CAA or AD), e.g., a level in a subject having AD or an amyloidosis. In some embodiments, once a subject is identified as having amyloidosis, e.g., AD or CAA, the methods can further include administering a treatment to the subject, e.g., a treatment as known in the art or as described herein. Treatments can include cholinesterase inhibitors, e.g., donepezil, rivastigmine, galantamine; NMDA antagonists, e.g., memantine; or monoclonal antibodies, e.g., aducanumab or an antibody as described herein. Alternatively or in addition, the methods can further include recommending or performing on the subject further diagnostic testing, e.g., mental status and neuropsychological testing, or imaging, e.g., using magnetic resonance imaging (MRI), or Computerized tomography (CT), or positron emission tomography (PET) scanning, e.g., Fluorodeoxyglucose (FDG) PET imaging, amyloid PET imaging, or Tau PET imaging.

As used herein the term “sample”, when referring to the material to be tested for the presence of a biological marker using the method of the invention, includes inter alia tissue, whole blood, plasma, serum, urine, saliva, breath, exosome or exosome-like microvesicles (U.S. Pat. No. 8,901,284), lymph, cerebrospinal fluid, or sputum. The type of sample used may vary depending upon the identity of the biological marker to be tested and the clinical situation in which the method is used.

The presence and/or level of oAβ can be evaluated using methods known in the art, e.g., using and quantitative immunoassay methods for proteins, including but not limited to, Western blot; enzyme linked immunosorbent assay (ELISA), e.g., sandwich ELISA; biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein a surface, e.g., beads or the wells of a mictrotiter plate, is coated with a capture antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, the presence and/or level of oAβ is comparable to the presence and/or level of the protein(s) in the disease reference, and the subject has one or more symptoms associated with amyloidosis, e.g., AD or CAA, then the subject has or can be diagnosed with amyloidosis, e.g., AD or CAA. In some embodiments, the subject has no overt signs or symptoms of amyloidosis, e.g., AD or CAA, but the presence and/or level of one or more of the proteins evaluated is comparable to the presence and/or level of the protein(s) in the disease reference, then the subject has an increased risk (above the level of risk of the general population) of developing amyloidosis, e.g., AD or CAA. In some embodiments, once it has been determined that a person has amyloidosis, e.g., AD or CAA, or has an increased risk of developing amyloidosis, e.g., AD or CAA, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of oAβ, e.g., a control reference level that represents a normal level of oAβ, e.g., a level in an unaffected subject or a subject who is not at risk of developing a disease described herein, and/or a disease reference that represents a level of the proteins associated with conditions associated with amyloidosis, e.g., AD or CAA, e.g., a level in a subject having amyloidosis, e.g., AD or CAA.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g., amyloidosis, e.g., AD or CAA).

A disease reference subject is one who has (or has an increased risk of developing) one or more of amyloidosis, e.g., AD or CAA. An increased risk is defined as a risk above the risk of subjects in the general population.

Thus, in some cases the level of oAβ in a subject being less than or equal to a reference level of oAβ is indicative of a clinical status (e.g., indicative of the absence of a disorder as described herein, e.g., amyloidosis, e.g., AD or CAA). In other cases the level of oAβ in a subject being greater than or equal to the reference level of oAβ is indicative of the presence of amyloidosis, e.g., AD or CAA, or increased risk of the disease. In some embodiments, the amount by which the level in the subject is the less than the reference level is sufficient to distinguish a subject from a control subject, and optionally is a statistically significantly less than the level in a control subject. In cases where the level of oAβ in a subject being equal to the reference level of oAβ, the “being equal” refers to being approximately equal (e.g., not statistically different).

The predetermined value can depend upon the particular population of subjects (e.g., human subjects) selected. For example, an apparently healthy population will have a different ‘normal’ range of levels of oAβ than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Also provided herein are kits for quantifying the amount of oAβ in a sample. The kits can include capture and detector antibodies, as described herein, optionally a positive control comprising, and optionally instructions for use of said reagents to quantify oAβ in the sample.

### Methods and Compositions for Treatment

Amyloidosis is a clinical disorder caused by extracellular and/or intracellular deposition of pathogenic amyloids in a variety of tissues. Signs and symptoms of amyloidosis may include: severe fatigue and weakness; shortness of breath; numbness, tingling, or pain in the hands or feet; swelling of the ankles and legs; diarrhea, possibly with blood, or constipation; an enlarged tongue, which sometimes looks rippled around its edge; or skin changes, such as thickening or easy bruising, and purplish patches around the eyes. Accumulation of amyloid-β peptide (Aβ) in the brain both in the form of plaques in the cerebral cortex and in blood vessel is associated with cerebral amyloid angiopathy (CAA) and Alzheimer's disease (AD).

There is strong evidence that soluble oligomers of amyloid beta protein (oAβ) help initiate the pathogenic cascade of Alzheimer's disease (AD), which suggests therapeutic strategies targeting oAβ over monomeric or fibrillar Aβ. The antibodies described herein, 71A1, is about 100-fold more sensitive for oAβ than synthetic monomers. The material that 71A1 specifically immunoprecipitates from AD soluble brain extracts impairs synaptic function as much as does the full extract. As shown herein, pre-incubating brain extracts with 71A1 neutralizes its synaptotoxicity. The unique activities of 71A1 against disease-relevant oAβ make it therapeutically useful for treating amyloidosis, e.g., AD or CAA.

Thus, provided herein are methods for treating disorders associated with accumulation of oAβ, e.g., amyloidosis, e.g., AD or CAA, in a subject. The methods include administering a therapeutically effective amount of 71A1 antibody, or an antigen-binding fragment thereof, to the subject, e.g., to the brain of the subject. As used in this context, to “treat” means to ameliorate at least one symptom of amyloidosis, e.g., AD or CAA. Often, amyloidosis results in impaired function in the affected organ; thus, a treatment can result in a reduction in oAβ and a return or approach to normal function. For example, AD and CAA are associated with progressive cognitive decline. Administration of a therapeutically effective amount of a compound described herein for the treatment of AD or CAA will result in decreased cognitive impairment, decreased rate of progressive cognitive decline, or cessation of progressive cognitive decline.

### Pharmaceutical Compositions and Methods of Administration

The methods described herein include the use of pharmaceutical compositions comprising or consisting of an antibody described herein, e.g., mAb 71A1 or 1G5, or antigen-binding fragments thereof, as an active ingredient. In some embodiments the antibody is a monoclonal antibody, non-human animal antibody, humanized antibody, chimeric antibody, human antibody, minibody, bispecific antibody, amino acid sequence-modified antibodies, modified antibody conjugated to other molecules (for example, polymers such as polyethylene glycol), or sugar chain-modified antibody that includes the HC and LC CDRs of mAb 71A1 or 1G5, or the entire HCs and LCs of mAb 71A1 or 1G5.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous delivery; intracerebroventricular, intracerebral, or intrathecal injection; or injection into CSF.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, the therapeutic compounds are prepared with carriers that will protect the therapeutic compounds against rapid elimination from the body, such as a controlled release formulation, including implants and microencapsulated delivery systems; the compounds can also be delivered, e.g., using a pump, e.g., a surgically implanted reservoir pump. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Nanoparticles (e.g., liposomes, polymeric nanoparticles, dendrimers, clathrin nanoparticles, or metallic nanoparticles), engineered bi-specific antibodies (e.g., that also bind to transferrin receptor (TfR), insulin receptor (IR), or the low-density lipoprotein receptor-related protein-1 (LRP-1), see, e.g., Faresjö et al., Fluids Barriers CNS. 2021; 18:26; Bajracharya et al., Pharmaceutics. 2021 December; 13(12): 2014), focused ultrasound in combination with microbubbles, and extracellular vesicles with blood-brain barrier (BBB)—crossing properties can be used to enhance delivery across the BBB. See, e.g., Bajracharya et al., Pharmaceutics. 2021 December; 13(12): 2014. Nanoparticles comprising liposomes can also be used as pharmaceutically acceptable carriers. These can be prepared according to methods known to those skilled in the art, for example, as described in U.S. Pat. No. 4,522,811Alternatively, viral vectors (such as AAV) encoding the antibodies can be delivered, e.g., comprising nucleic acids (preferably codon-optimized for use in humans) encoding a therapeutic antibody as described herein.

The pharmaceutical compositions can be included in a container, pack, or dispenser together with instructions for administration.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Example 1. Peptide Immunogens

The human Amyloid b-protein 42 (Aβ42) amino acid sequence and the region within the sequence (aa 9-18) used to generate immunogenic peptides is as follows:

(SEQ ID NO: 39)

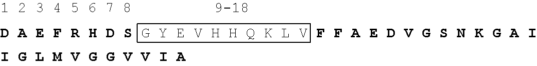

This sequence was chosen because of the cluster of amino acids that contain complex side chains. The thought was that when the amino acids aligned in dimer, trimer, and even more complex structures, they might re-associate into unique orientations that create distinctive, three dimensional structures similar to those that may form early in Aβ aggregation. These structures may be “neo-epitopes” for antibody binding that are not present in the unaggregated, monomer sequence. The main thrust of the immunogen design was to create a molecule, a stable dimer ideally but also a stable trimer or even stable 4 peptide structure that would restrict as much as possible the focus of the immune response to conformational neo-antibody epitopes created when Aβ initially aggregates that are subsequently lost when the larger aggregates form.

A synthetic peptide version of this region was crafted for use as an immunogen in Balb/c mice, with the sequence

(SEQ ID NO: 2)
CGKCGYEVHHQKLVPNVLKQHHVEYGCK.

A schematic illustration of the cyclized peptide is shown in FIG. 1. Both Mab 71A1 and 1G5 were isolated using this immunogen conjugated to a proprietary T cell epitope peptide.

This immunogen, also referred to herein as NETL-1, was derived from the 10 amino acid stretch within Aβ sequence depicted above. This region was from a sequence thought possibly involved in the first conversion from an alpha helical to a β-sheet conformation, similar to the β-sheet conversion that is needed to form Aβ protofibril aggregation. The 10 amino acid stretch was repeated backwards after the addition of a proline and an asparagine to form a kink when the peptide is allowed to cyclize before cleavage from the resin. The N terminal cysteine was employed to conjugate the fully synthesized and resin-cleaved peptide to sulfhydryl-reactive ELISA plates and to a carrier protein such as maleimide-activated KLH (Keyhole Limpet Hemocyanin) or to a proprietary maleimide-peptide carrier designated Tepi2. Maleimide is sulfhydryl-reactive, and cysteines have free sulfhydryl groups available for conjugation to other sulfhydryl groups or maleimide. An additional Lysine at the N terminus of the peptide was added to increase water solubility. Control peptides for screening included both full length Aβ40

(DAEFRHDSGYEVHHQKLVFFAEDVGSNKGAIIGLMVGGVV, SEQ ID NO: 40)

with a C-terminal cysteine as well as NETL-2 peptide, the same Aβ 10 amino acid peptide sequence used in NETL1 but without the repeat in reverse and with an additional N terminal lysine and cysteine.

Tepi2 is a sequence that when presented in the context of class II MHC antigens on antigen presenting cells (APC) activates helper T cells to initiate the antibody response to the Abeta sequence structure. An exemplary Tepi2 is as follows:

```
(SEQ ID NO: 4)
                        PamC3
                          |
Maleimide-KSSKSKKKFISEAIIHHLHSRHPGK
```

It is a peptide sequence (SEQ ID NO:4) with a Pam3Cys (N-α-Palmitoyl-S-2,3-bis(palmitoyloxy)-(2RS)-propyl-L-cysteine, Palmitoyl-Cys((RS)-2,3) attached to the second lysine (K4). Pam3Cys is a lipopeptide adjuvant known to stimulate innate immune responses. Innate immune responses are those that utilize germline-encoded pattern recognition receptors (PRRs) that bind to components, similar to Pam3Cys, that are found on many microbes and parasites. Induction of the innate immune system results in the production of many inflammatory cytokines and can trigger immediate host defensive responses to prime antigen-specific adaptive immune responses such as the induction of specific antibody production. (Janeway C A Jr, Medzhitov R. Innate immune recognition. Annu Rev Immunol (2002) 20:197-216).

The purpose of attaching Tepi2 to the peptide immunogens rather than continue with the large protein carrier was twofold. First, large protein carriers themselves induce antibody responses and many of the activated B cells are responses to unwanted and irrelevant epitopes found on these proteins. Attaching the Tepi-2 carrier to subsequent boosts therefore limited continued activation of primed B cells specific to the desired Abeta peptide immunogen. Secondly, antibody responses to the additional amino acid composition of Tepi2 could never be measured in our hands by ELISA with Tepi2 coated plates.

### Example 2. Biochemistry and Immunology

NETL-1 contained three cysteines with available sulfhydryls for conjugation and for post-synthetic modifications prior to cleavage from the resin used to produce the peptide (Anaspec, Inc). Peptides were synthesized using FMOC chemistry in an automated peptide synthesizer such as the Applied Biosystems 432A. The peptides have side chain protecting groups (Trt—tert-butyloxycarbonyl or Boc) that are necessary to guarantee the integrity of the amino acid through the synthetic process. These groups must be removed when the finished product is cleaved from the resin that is used to synthesize the peptide. In NETL-1 there are two inserted cysteines on both c-termini of the peptide (C4 and C27). These cysteines were used to cyclize the peptide on resin and before final cleavage from the resin. To accomplish this task the cysteines were protected with an acetamidomethyl (ACM) group. Using a Thallium III procedure to remove the ACM groups, these cysteines were preferentially cleaved without affecting the Boc protecting groups that are removed under different conditions used to cleave the peptide from the resin. Removal of the ACM protecting group is believed to generate free sulfhydryls that should preferentially react with one another to form a loop on the resin. It is also possible that some dimers between the peptides will also form. The scheme, of course, to fashion stable dimer structures using these synthetic acrobatics that create potential βeta sheet structures, precludes neither bonding preference. Additional amino acids after the C-terminal valine in the first Abeta 9-18 sequence or on either the N or C-terminus of the immunogen may accomplish the same goal to form stable small Abeta structures formed early in aggregation. The isolated 71A1 and 1G5 antibodies responsive to tertiary structures and not the native, contiguous, and “linear’ sequence was then teased out of a responding repertoire toward many different configurations. After final cleavage from the resin the likely result is a stably associated dimmer with a free sulfhydryl available at the N terminal of the peptide for conjugation.

There are additional amino acid protective groups that can be used to create stable dimerization within a peptide sequence. For example, the Monomethoxytrityl (MMT) protecting group is a versatile amino protecting group that is easier to remove because it is more sensitive to strong acids, bases and nucleophiles than is the ACM protecting group. The 10 amino acid stretch used in NETL-1 can also be repeated in tandem separated by a proline and an asparagine as depicted below:

(SEQ ID NO: 3)
C G K C G Y E V H H Q K L V P N G Y E V H H Q K L V C K

The first cysteine (C1) could be protected by the TRT group used with all the amino acids during peptide synthesis and cleavage from the resin. Cysteine No. 2 (C4) could be protected by, for example, an ACT group and cysteine No. 3 (C27) with an MMT group. The MMT group would be removed first to allow for disulfide bonding between adjacent peptides through C4 disulfide bridging and the ACT group subsequently removed to allow for an additional C27 disulfide bridge between the same adjacent peptides. When the peptides are cleaved from the resin, stable peptide structures with adjacent repeating dimers could be produced:

(both are SEQ ID NO: 3)
C G K C G Y E V H H Q K L V P N G Y E V H H Q K L V C K
| |
C G K C G Y E V H H Q K L V P N G Y E V H H Q K L V C K

Other protecting groups and strategies can be used in this and in the NETL-1 peptide synthetic process with the same goal in mind of creating stable dimer, trimer or 4 peptide aggregates with unique three dimensional structures not available on the monomer Aβ40 or Aβ42 peptide.

Normal immune responses involve activation of both B and T cell subsets of lymphocytes. A response initiated against a complex protein requires the intake and digestion followed by cell-surface presentation of peptide fragments derived from this protein. These fragments are presented to the intracellular environment in association with cell surface-expressed molecules called MHC molecules on the B cells and specialized cells called APC or Antigen Presenting Cells. T cells have specialized receptors that recognize these peptide fragments in associated with the MHC. This recognition initiates intercellular events inside the T cell that lead to the production and elaboration of cytokines. These cytokines stimulate B cell maturation and ultimately to the development antibody-secreting B cells.

A large body of work exists that seeks to understand and elucidate the process and mechanism of T cell activation. The sequences within proteins that are involved in T cell activation are called T cell epitopes and the identification of these sequences and characteristics among them an area of interest for immunologists. Normally, if antibody responses to a small peptide are sought, the peptide is conjugated to a large carrier molecule such as KLH or BSA. This conjugation process allows B cells and APC to internalize, process and present T cell epitope peptides that are derived from the large carrier protein. The problem with these carriers, however, is that they also have a large number of B cell epitopes and more often than not the antibody responses to these regions swamp the response to the peptide that is conjugated to them. The result is a decrease in specific titer to the peptide of interest over time. This phenomenon does not occur with the use of a T cell epitope because the only B cell epitopes available for recognition come from the peptide or molecule conjugated to them.

The T cell epitopes used to stimulate Mab 71A1 and 1G5 are sequences that we have used to initiate mouse antibody responses to small molecules and peptides, in particular. These sequences, for example, conjugated to small peptide immunogens that are themselves derived from a protein of interest, will generate antibody responses in the mice against the peptide of interest. No antibody responses directed against the T cell epitopes are observed. In addition, the inventors have undertaken studies in the past that show that these sequences alone will lead to T cell proliferation. In this sense, these T cell epitopes will, in all likelihood, associate with the MHC on B cells and APC to present a complex that is recognized by T cells. This recognition completes the initiation requirement that will lead to antibody responses against the molecule that is conjugated to these T cell epitopes.

NETL-1 peptide was conjugated to the T cell epitope peptide at a 1:1 molar ratio in 0.1M sodium phosphate buffer pH 6.5 containing 5 mM EDTA to reduce any disulfide bonding between the NETL-1 peptides. Both Control Aβ42-Cysteine and NETL-2 were treated similarly.

The complete cyclization and cleavage protocol is as follows. First, determine the mass of peptide resin that will be cyclized, and determine the mass of thallium III required for cyclization: (X mM NETL12×2)×543.2 mg/mM=Y mg thallium III. Swell the resin in a glass vial using DMF. Transfer required volume thallium III to peptide resin (same quantity as added to swell peptide-resin). Allow to react with stirring for 2 hr at 0° C. Transfer reaction mixture to an appropriate size glass sintered filter flask for filtration. Wash the peptide-resin mixture three times with DMF, and then wash the peptide-resin mixture three times with DCM.

Cover sintered flask and dry the mixture overnight. Transfer the dried peptide-resin to a glass vial with cover and determine yield.

The following day, weigh out the cyclized peptide-resin into an appropriately-sized glass vial containing a magnetic flea or stir bar may.

Prepare a cleavage cocktail comprising thioanisole, deionized water, ethanedithiol, and trifluoroacetic acid and chill on ice. Slowly add the entire volume of chilled cocktail to vial containing peptide-resin and stir on ice for fifteen minutes then at room temperature for 165 minutes.

During cleavage reaction, chill MTBE on ice. For each 100-150 mg peptide-resin, use approximately 35 ml of cold MTBE. Run the peptide-resin-cocktail mixture over a Quik-Sep column into the cold MTBE. Wash with TFA. Centrifuge to pellet the cleaved peptide, then wash/resuspend the peptide pellet in cold MTBE; repeat centrifuge and wash three times

Centrifuge to pellet peptide as per previous spin, and dry briefly. Dissolve pellet in minimal volume of glacial acetic acid. Freeze and lyophilize once or twice.

### Example 3. Immunization and Antibody Screening

Balb/c Mice were given a primary and secondary immunization with KLH-conjugated NETL-1 peptide. Subsequent boosts were undertaken at three-week intervals with T cell epitope-conjugated peptide. This approach allowed KLH to initially establish a strong immune response to the peptides. Before antibody responses against KLH began to swamp responses to the peptide the T cell epitope allows for further boosting against the structures of interest in the absence of further induction of irrelevant antibody responses that result from B cell epitopes contained within KLH. The T cell epitope induces minimal to no B cell responses but does induce a strong T cell proliferative response important for B cell induction and maturation.

The rationale behind the NETL-1 immunogen was that a more focused epitope formed by a cyclized peptide may restrict the response to a dimer structure derived from the chosen 10aa sequence with minimal or no induction of responses to the monomer 10aa peptide sequence found within full length Aβ40. Both responses were observed. We can account for this, in part, by the fact that there may always be a minimal epitope available for recognition that is cross-reactive with the monomer sequence no matter how small the peptide structure. Several monoclonal antibodies specific for both NETL-1 and NETL-2 were isolated. Two isolates, 7a and 1g stood out that bound to NETL-1 but not to NETL-2 or Aβ40. These isolates were sub-cloned for further characterization. Two isolated subclones, Mab 71A1 and 1G5 were chosen, grown in tissue culture and purified for confirmation of specificity and for future studies. Both 71A1 and 1G5 monoclonal antibodies bound to the cyclized peptide, NETL-1 and not to control wells, Aβ40 monomer peptide or control peptide NETL-2 (FIG. 2). FIG. 2 is a standard, indirect enzyme linked immunosorbent assay (ELISA) with the purified monoclonal antibodies assayed for binding to Aβ40, NETL 1 or NETL 2 peptides conjugated to malemide-activated (SH-reactive) micro titer plates. After one hour incubation, the wells were washed and then 100 ul of a secondary, Horseradish peroxidase (HRPO)-conjugated goat anti-mouse antibody was added and the plates, incubated for an additional hour before addition of TMB (3,3′,5,5′-Tetramethylbenzidine) substrate. Absorbance was measured in a spectrophotometer at 650 nm.

### Example 4. Ultra-Sensitive Immunoassays to Detect and Quantify Soluble as Oligomers in Human Plasma

This example describes a sensitive, high throughput and inexpensive method to quantify synaptotoxic oAβ in human plasma for analyzing large cohorts of aged and AD subjects to assess the dynamics of this key pathogenic species and response to therapy.

### Methods

The following methods were used in Example 4.

### Generation of Aβ Oligomer Specific Antibodies 71A1 and 1G5

Monoclonal antibodies 71A1 (a subclone of parent clone 7A1a) and 1G5 were raised against a synthetic conformational peptide immunogen designed to potentially mimic the three-dimensional structure formed upon dimerization of the monomeric Aβ. The peptide immunogen was synthesized containing amino acid residues 9-18 of Aβ₁₋₄₀ and post-synthetically modified to cyclize and allow folding into a stable dimer formation (patent pending). The rationale behind the selection of residues 9-18 of the Aβ peptide was that the amino acids in this region would likely re-associate into a unique three-dimensional structure only present in aggregated forms of Aβ. Balb/c mice were immunized with this conformational peptide immunogen using a maleimide-activated keyhole-limpet-hemocyanin (KLH) carrier complex for primary and secondary immunizations and a proprietary T helper 2 cell epitope for subsequent boosts prior to the splenectomy procedures. Fusions were performed using immunized mouse splenocytes and mouse myeloma F0 cells as the fusion partner. After primary screening with the immunogen and subcloning, two monoclonal candidates, 71A1 and 1G5, in particular, showed high specificity to cyclized peptide and no specificity to linear peptide. Initial characterization of these antibodies had demonstrated high specificity for synthetic and endogenous oligomeric forms of Aβ and no binding to monomeric Aβ [44].

### Reagents

Synthetic β-amyloid peptides 1-40 and 1-42 were purchased from Anaspec. Aβ 1-40 in which serine 26 was substituted with cysteine (Aβ S26C) were purchased from the Keck Biotechnology Center (Yale University, New Haven, CT). Amyloid-β derived diffusible ligands (ADDLs) [1] and S26C-dimer were prepared as per previous reports [45].

### Mice

Both male and female C57BL/6J mice were used. All procedures involving mice were in accordance with the animal welfare guidelines of Harvard Medical School and Brigham and Women's hospital.

### Preparation of Soluble Brain “Soaking” Extracts

Extraction of soluble protein from postmortem brain tissue using the “soaking” method was performed as described previously[11]. Briefly, cortical grey matter was dissected from freshly thawed coronal slices, then chopped at 0.5-mm-wide intervals on a McIlwain tissue chopper. Chopped tissue bits were weighed and added at 1:5 w:v to extraction buffer (25 mM Tris, 150 mM NaCl, 5 μg/ml leupeptin, 5 μg/ml aprotinin, 2 μg/ml pepstatin, 120 μg/ml Pefabloc, 5 mM EDTA, 5 mM NaF, pH 7.2). Tissue bits were soaked by nutating at 4° C. in 50-ml Eppendorf protein LoBind tubes for 30 min. Tissue bits were removed by spinning for 10 min at 4° C. at 2,000 g in a Fiberlite F14-14×50cy rotor in a Sorvall Lynx 6000 tabletop centrifuge (Thermo Fisher). The upper ˜90% of supernatant was removed and then spun in an SW41Ti rotor in an Optima L90K ultracentrifuge (Beckman Coulter) at 40,000 rpm for 110 min at 4° C. The upper ˜90% of supernatant was retained as the “soaking extract” for further studies. Brain tris-buffer saline (TBS) soaking extracts were aliquoted into 1.5-ml Eppendorf Protein LoBind tubes and stored at −80° C.

### Human Biofluids and Brain Tissue

Human CSF and plasma were obtained from two clinical cohorts, one from the BWH Division of Cognitive and Behavioral Neurology, and one from the Mayo Clinic Study of Aging. Table 1 shows demographic characteristics, and further details of both cohorts. The BWH patients referred for diagnostic lumbar puncture were consented for donation of a plasma sample and extra CSF and access to their medical records under BWH IRB approval. Blood was collected into EDTA tubes, centrifuged at 1500 g for 15 min, and the plasma aliquoted and frozen at −80° C., all within 3 h of collection. CSF was drawn directly into polypropylene tubes (Sarstedt). A portion of CSF was sent to Athena Diagnostics for the ADmark Panel (consisting of Aβ₁₋₄₂, T-tau, and P-tau) and the remainder frozen immediately on dry ice, then thawed and aliquoted at a later date. Clinical diagnostic information was obtained through chart review by a board-certified behavioral neurologist prior to the ADmark or research ELISA results being known.

The Mayo Clinic Study of Aging (MCSA) is a population-based, prospective study of residents living in Olmsted County, MN. Details of study design and participant recruitment are published [46, 47]. In 2004, the Rochester Epidemiology Project (REP) medical records linkage system was used to enumerate Olmsted County residents between the ages of 70 and 89, as described [48]. In 2012, the MCSA was extended to include those aged 50 and older. The present analyses included 73 Mayo participants in whom we measured plasma Aβ oligomers, as described herein. Subject enrollment, sample collection and sharing of samples across sites was approved by the Mayo Clinic and the Olmsted Medical Center. The institutional review boards of the Mayo Clinic and the Olmsted Medical Center approved study protocols and written informed consent was obtained from all participants.

Human brain tissue was obtained at BWH or MGH from deceased donors with probable AD undergoing diagnostic autopsy. One hemisphere was fixed for diagnostic purposes and one hemisphere was sliced coronally and frozen at −80° C. All human subjects research received prior approval by the Mass General Brigham Institutional Review Board, and informed consent was obtained for all human subjects.

TABLE 1
Patient demographics in current study

| | | | | Most clinically |
|---|---|---|---|---|
| | Age | Sex | | suspected |
| | (mean +/− | (% | Functional category | neuropathology |
| Cohort | SD) | male) | (N, %) † | (N, %) |
| BWH | 65.9 +/− 8.3 | 38.9 | Unimpaired (2, 5.6); | AD (23, 63.9); LBD |
| (CSF) | | | Subjective cognitive | (2, 5.6); FTLD |
| | | | decline (1, 2.8); MCI | (2, 5.6); |
| | | | (19, 52.8); Dementia | None (6, 16.7); |
| | | | (14, 38.9); Unknown | Unknown (3, 8.3) |
| | | | (2, 5.6) | |
| Mayo | 74.4 +/− 8.9 | 53.4 | Cognitively normal | Normal (73, 100) |
| Clinic | | | (73, 100) | |
| (Plasma) | | | | |

†Totals exceed 100% because some subjects were on the cusp of the MCI or dementia category and were counted twice, once per category.

### Electrophysiological Extracellular Field Recordings

Experiments were performed as previously described [13]. Briefly, mice (1-3 mo) were deeply anesthetized with halothane and decapitated. Transverse acute hippocampal slices (350 μm) were cut in ice-cold oxygenated sucrose-enhanced artificial cerebrospinal fluid (aCSF) containing 206 mM sucrose, 2 mM KCl, 2 mM MgSO₄, 1.25 mM NaH₂PO₄, 1 mM CaCl₂), 1 mM MgCl₂, 26 mM NaHCO₃, 10 mM D-glucose, pH 7.4. After dissection, slices were incubated in aCSF that contained the following (in mM): 124 NaCl, 2 KCl, 2 MgSO₄, 1.25 NaH₂PO₄, 2.5 CaCl₂), 26 NaHCO₃, 10 D-glucose saturated with 95% 02 and 5% C02 (pH 7.4), in which they were allowed to recover for at least 90 min before recording. Recordings were performed in the same solution at room temperature in a chamber submerged in aCSF. To record field EPSPs (fEPSPs) in the CA1 region of the hippocampus, standard procedures were used. Test stimuli were applied at low frequency (0.05 Hz) at a stimulus intensity that elicited a fEPSP amplitude that was 40-50% of maximum, and the test responses were recorded for 10 min before the experiment was begun to ensure stability of the response. Once a stable test response was attained, for experimental treatments, 0.5 mL AD brain TBS soaking extracts were added to the 9.5 mL aCSF perfusate, and a baseline was recorded for an additional 30 min. For the anti-Aβ antibody experiments, the 71A1 antibody was added to an aliquot of AD brain extract and incubated with mixing for 30 min, then the mixture was added to the brain slice perfusion buffer. To induce LTP, two consecutive trains (1 s) of stimuli at 100 Hz separated by 20 s were applied to the slices. Traces were obtained by pClamp 11 and analyzed using Clampfit 11. Data analysis was as follows. The fEPSP magnitude was measured using the initial fEPSP slope, and 3 consecutive slopes (1 min) were averaged and normalized to the mean value recorded 10 min before the conditioning stimulus. Data are presented as means f SEMs. Significant differences were determined using a one-way ANOVA test with post hoc Tukey's test.

### Electrophoresis and WB

Samples were loaded onto 4-12% or 12% Bis-Tris gels (SurePAGE, Genscript) using MES-SDS running buffer (Invitrogen), transferred to nitrocellulose membranes, and probed for various proteins using standard WB. The resultant blots were detected by ECL and signals were captured by film.

### Immunoprecipitation

800 μL human brain TBS soaking extracts or CSF samples were incubated with 10 μg antibodies for 1 hr at 4° C. The immunoprecipitates (IPs) were incubated with Protein G Magnetic Beads (Bio-Rad) at 4° C. overnight and then washed three times in TBS. Post-IP solutions were also saved for analysis. The immunoprecipitated proteins were then eluted by 8M guanidine hydrochloride (GnCL) (Thermo-Fisher).

### Size-Exclusion Chromatography (SEC)

Brain TBS soaking extracts or CSF (350 μL total volume) were injected onto a Superdex 200 increase and run on a fast protein liquid chromatography (FPLC) system (AKTA; GE Healthcare) in TBS, pH 7.4. 500 μL fractions were collected for downstream experiments. Columns were calibrated with Gel Filtration Standards (Bio-Rad), which range from 1,350 to 670,000 Da.

### Affinity Purification Using 71A1

Brain TBS soaking extracts (2 mL) were mixed with pre-conjugated High-Capacity Streptavidin Agarose resin (Pierce) with 50 ug biotinylated 71A1 for 2 hr at room temperature (RT) under nutation. Beads were extensively washed with 10 column volumes of SMCxPRO wash buffer and eluted with 0.2 M Glycine (pH 3) followed by neutralization with 1 M Tris-HCl (pH 8.5). Affinity purified material was desalted with PD MidiTrap G-25 into PBS, pH 7.4, before application on hippocampal slices.

### MSD ELISA

Human brain extracts, CSF, immunoprecipitated samples and SEC fractions were each diluted with 1% BSA in wash buffer (TBS supplemented with 0.05% Tween). For our home-made Meso Scale Discovery assay (MSD) electrochemiluminescence platform, each well of an uncoated 96-well multi-array plate (Meso Scale Discovery, #L15XA-3) was coated with 30 μL of a PBS solution containing capture antibody (3 μg/mL 266, a monoclonal antibody recognizes mid-region of Aβ [murine analog of Solanezumab], for all human Aβ ELISAs) and incubated at RT overnight followed by blocking with 5% BSA in wash buffer for 1 h at RT with shaking at >300 rpm. A detection antibody solution was prepared with biotinylated detection antibody, 100 ng/mL Streptavidin Sulfo-TAG (Meso Scale Discovery, #R32AD-5), and 1% BSA diluted in wash buffer. Following the blocking step, 50 μL/well of the sample, followed by 25 μL/well of detection antibody solution were incubated for 2 h at room temperature with shaking at >300 rpm, washing wells with 150 μL wash buffer between incubations. The plate was read and analyzed according to the manufacturer's protocol. The antibodies used to detect specific antigens were for hAβ (1-40 specific), 139-5, (rabbit recombinant, Biolegend); and for hAβ (1-42 specific), D3E10 (rabbit recombinant, Cell Signaling Technology).

### SMCxPRO Immunoassay

The SMCxPRO (platform Sigma Millipore) is based on single-molecule-counting technology and typically allows a 20- to 100-fold increase in sensitivity compared with traditional detection systems. Biotinylated capture mAbs (1C22, 1G5 and 71A1) were conjugated to streptavidin magnetic particles (MPs) (Dynabeads MyOne, Thermo Fisher Scientific) at a ratio of 12.5 μg biotinylated antibody per milligram of MPs using a kit from Sigma Millipore. MPs with bound capture mAb were diluted to 50 μg/mL in the Aβ Oligomer Assay Buffer (Tris buffer; 50 mM Tris, 150 mM NaCl, pH 7.6), with 1% Triton X-100, 0.0005% (w/v) d-des-thio-biotin and 0.1% bovine serum albumin), and 50 μL of this suspension was added to 150 μL of sample, standard or blank and incubated at 600 rpm on a shaking incubator at 25° C. for 2 h. MPs were isolated using a magnet and unbound material was removed by washing with 1×SMC wash buffer using a HydroFlex plate washer (Tecan Group AG, Männedorf, Switzerland). Fluorescently labeled (Alexa-647 dye) detection antibody 3D6 (20 μL, 200 ng/mL) was added to each well. MPs bearing the antibody-oligomer Aβ sandwich were then incubated with agitation using a Jitterbug shaker (Boekel, Feasterville, PA, USA) for 1 h at 25° C. Unbound detection reagent was removed by washing 4 times with the wash buffer. The wash buffer was removed by aspiration, and fluorescently labeled 3D6 detection antibody was released by shaking in an Elution Buffer B (11.5 μL/well) for 10 min at 25° C. 11 μL of the eluates were then transferred to the wells of a clean 96-well plate containing the Neutralization Buffer D (11 L/well). The neutralized sample (20 L/well) was then transferred to a black 384-well-read plate (Aurora) and read by the SMCxPRO instrument. When fluorescently labeled antibodies are excited by a 642 nm laser and pass through the interrogation space, they emit light that is measured using a confocal microscope lens and a photon detector. The output from the detector is a train of pulses, with each pulse representing one photon that was detected. The lower limit of reliable quantification (LLoQ) was defined as the lowest back interpolated standard that provides a signal two-fold the background with a percentage of recovery calculated between 80% and 100% and coefficient of variance (CV)≤20%.

### Immunohistochemistry

Fresh or thawed (from frozen stock) human brain blocks were embedded in Tissue-Tek® O.C.T. Compound and frozen at −80° C. overnight. Before sectioning at 20-30 μm thickness using a cryostat (Leica), frozen blocks were changed to −20° C. for 2 h to soften tissue for sectioning. Cryo-sections were then directly mounted on MAS-GP™ Adhesion Microscope Slides (Matsunami) and stored at 4° C. before staining. For 3,30-diaminobenzidine (DAB) staining, cryo-sections were equilibrated in PBS containing 0.3% Triton-X100 (PBST) for 30 min followed by blocking endogenous peroxidase activity and antibody non-specific binding, for 1 h respectively. Primary antibodies were diluted in PBST and incubated with sections at 4° C. overnight. After 3 washes with PBST, sections were then incubated with biotinylated secondary antibodies for 1 h. The immunoreactive products were visualized by incubating with 1) DAB containing nickel ammonium sulfate as an enhancing reagent or 2) Vina Green chrome (Biocare Medical). Stained sections were observed using an Axioskop 2 (Zeiss).

### Quantification and Statistical Analysis

All statistical analysis was performed using GraphPad Prism 9 software. Statistical details of experiments are described in the text and/or figure legends.

### Example 4.1 Immunogen Design and Monoclonal Antibody Generation of 71A1 and 1G5

Monoclonal antibodies 71A1 (a subclone of parent clone 7A1a) and 1G5 were raised against a synthetic conformational peptide immunogen designed to mimic the potential three-dimensional structure formed upon the dimerization of the monomeric amyloid β-protein (Aβ). The peptide immunogen was synthesized to contain amino acid residues 9-18 of Aβ₁₋₄₀ and post-synthetically modified to cyclize and allow folding into a stable dimer-like formation (patent pending). The rationale behind the selection of residues 9-18 of the Aβ peptide was that the amino acids in this region would likely re-associate into a unique three-dimensional structure only present in aggregated forms of Aβ.

Balb/c mice were immunized with this cyclized conformational peptide using a maleimide-activated keyhole-limpet-hemocyanin (KLH) carrier complex for primary and secondary immunizations and a proprietary T helper 2 cell epitope for subsequent boosts prior to the splenectomy procedures. Fusions were performed using immunized mouse splenocytes and mouse myeloma F0 cells as the fusion partner. After primary screening with the immunogen and subcloning, two monoclonal antibody candidates in particular (71A1 and 1G5) showed high specificity to cyclized peptide and no specificity to linear synthetic peptide. Initial characterization of these antibodies had demonstrated high specificity for synthetic and endogenous oligomeric forms of Aβ and no binding to monomeric Aβ [44].

### Example 4.2 71A1 and 1G5 Recognize Aβ in Human Brain Biochemically and Histologically

We tested these two novel antibodies, 1G5 and 71A1, on natural sources of human Aβ. First, we used a recently developed method [11] called “soaking extraction” to obtain highly diffusible Aβ species from minced brain bits of neuropathologically typical AD cortex incubated for just 30 minutes in Tris-buffered saline (TBS) without homogenization. These diffusible aqueous extracts have been shown to retain most of the synaptotoxic activity of AD cortical samples; subsequent homogenization of the brain bits post-soaking yields much more Aβ, but this has little synaptotoxic activity [11]. The soaking procedure is illustrated in FIG. 3A. We used 1G5 and 71A1 to conduct immunoaffinity pulldown from AD brain soaking extracts, alongside positive control antibodies 4G8 (principally anti-monomer) and 1C22 (anti-oligomer) and as a negative control normal murine IgG. Compared to 4G8 and 1C22, both 1G5 and 71A1 pulled down far less Aβ from the AD brain soaking extracts but consistently more than murine IgG which pulled down no Aβ, as judged by immunoblots probed with 2 rabbit monoclonal recombinant antibodies (mAb) raised against different Aβ epitopes (FIG. 3B). Because in denaturing SDS-PAGE, soluble oAβ are disassembled into monomers and some covalently bonded dimers [49], we observed immunoblot signals around and below ˜6-7 kDa (FIG. 3B, lower panel (longer exposure)). To confirm this finding, we then used our highly specific home-brew Aβ x-40 and x-42 monomer immunoassays [50, 51] to quantify the Aβ contents pulled down from three individual AD brains by 1G5, 71A1, 1C22 (positive control) and murine IgG (negative control). As shown in FIG. 3C (Aβ pulled down with protein G beads, then denatured by guanidine hydrochloride (GnCl)) and FIG. 3D (Aβ remaining in the post-IP supernatant, then denatured by GnCl), both 1G5 and 71A1 pulled down small but consistent amounts of Aβ from AD brain soaking extracts, always more than the negative-control murine IgG but far less than did 1C22, a result consistent with the immunoblot data (FIG. 3B). Also, to demonstrate the consistency of the pulldowns and immunodepletion by the 4 antibodies, we calculated the Aβ mass (in ng, measured against an ADDL standard) obtained in the IPs and post-IPs by the 4 antibodies from 3 AD brains.

After thus establishing that 1G5 and 71A1 can bind Aβ from highly soluble human brain extracts, we asked whether 1G5 and 71A1 can label Aβ plaques in the human brain using immunohistochemistry. When we first tested 1C22 for its use in immunohistochemistry, we found that it could not stain typical PFA-fixed AD brain sections but could readily stain unfixed cryo-sections (FIG. 4A). Moreover, 4% PFA treatment of the latter sections for 20 minutes diminished most of the 1C22 immunoreactivity (FIG. 4A). This phenomenon is common among antibodies that recognize a conformational epitope. For 1G5 and 71A1, we found that 1) both antibodies labeled extracellular amyloid plaques (i.e., deposited Aβ) in unfixed cryosections with similar patterns; and 2) 20-minute 4% PFA treatment substantially diminished the labeling (FIG. 4A).

To establish further that these antibodies labeled parenchymal Aβ, we utilized double immunostaining: sequentially labeling cryo-sections with D54D2 (to the Aβ N-terminal region, representing total Aβ visualized by DAB) and then either 1C22 or 71A1 (visualized by Vina Green). In FIG. 4B, the left panels show that D54D2 alone labeled amyloid plaques; the middle panels show amyloid plaques double-labeled by D54D2 for total Aβ (DAB) and 1C22 or 71A1 (vina Green); and the right panel shows the double-labeled plaques at higher magnification. These findings confirm that 71A1 and 1G5 recognize Aβ brain deposits in situ. We also tested 1C22, 1G5 and 71A1 on a control brain devoid of plaques to show their specificity towards Aβ, and all 3 antibodies failed to detect any signals (data not shown).

As the immunoreactivity of 1G5 and 71A1 were similar to the previously demonstrated oligomer-preferring 1C22 [52, 53], we next asked if the two new antibodies could protect against Aβ-induced synaptic toxicity of soluble AD brain extracts. Using electrophysiology of wild-type (wt) mouse brain hippocampal slices [54], we found that 71A1 added to the slice perfusate at 2.12 μg/mL fully prevented the inhibition of hippocampal LTP by AD soaking extract while having no effect by itself on LTP (FIG. 4C). Specifically, fEPSP slope in artificial cerebrospinal fluid (aCSF) vehicle alone was 152.6±5.3% (N=6); in aCSF+AD extract: 115.7±4.9% (N=5); in aCSF+71A1 alone: 149±8.3% (N=4); and in aCSF+AD extract pre-mixed with 71A1: 154.9±9.8% (N=4). This effect was closely similar to the oAβ-neutralizing benefits of mAbs 3D6 and 82E1 we previously reported [13]. Furthermore, we used 71A1 to affinity purify oAβ from the same AD brain soaking extract and tested the synaptotoxicity of the purified oAβ on wt mouse brain hippocampal slices. The affinity-purified oAβ also significantly suppressed hippocampal LTP (FIG. 4D). Statistical analyses of the differences 1) between AD brain extract alone and AD brain extract pre-mixed with 71A1 confirmed that 71A1 significantly rescued the LTP deficits caused by the extract (FIG. 4E), and 2) between 71A1-purified oAβ and aCSF control showed that 71A1-purified oAβ significantly inhibited LTP, similar to the input soaking extract (FIG. 4F). Collectively, the data indicate that the 71A1-reactive Aβ species, although a minor population of oAβ in AD brain as shown by immunoprecipitation with protein G (FIGS. 3B-D), confers synaptotoxicity that can be neutralized by 71A1.

### Example 4.3 71A1 and 1G5 Bind Aβ Species Preferably in CSF Over Brain, the Converse of 1C22

Next, we asked to what extent 1G5 and 71A1 could recognize Aβ from another natural source, human CSF. Using a similar experimental setup as that employed affinity pulldown from brain soaking extracts (FIG. 3C, D), we used 1G5, 71A1, 1C22 and murine normal IgG (control) to pull down from three individual CSF samples from AD patients collected at the Memory Disorders Clinic at Brigham and Women's Hospital. 1G5 and 71A1 each immunoprecipitated surprisingly high amounts of Aβ, as measured by Aβ x-40 and x-42 monomer immunoassays after GnCL denaturation (FIG. 5A, upper panel), with corresponding decreases in the post-IP supernatants (FIG. 5A, lower panel). Moreover, in contrast to the soluble AD brain extracts studied above, both 1G5 and 71A1 pulled down higher relative amounts of Aβ x-42 from CSF than did 1C22, suggesting a possible difference in the native Aβ populations between human brain and CSF. In all three CSF samples, we also found that 71A1 showed a greater Aβ pull-down efficiency than 1G5 (FIG. 5A). Therefore, we decided to use 71A1 to perform IP-ELISAs on 19 different CSFs from the same BWH clinic (see Table 1 for patient demographics and diagnoses). 71A1 immunoprecipitated Aβ from all of the CSF samples (FIG. 5B). Next, we asked if this IP-ELISA data correlated with the commercial ADmark Aβ 1-42 levels obtained in the same CSFs by our clinic. The Aβ x-42 signal (post-GnCl denaturation) from our 71A1 immunoprecipitations (IPs) correlated significantly with ADmark CSF Aβ 1-42 levels across our 19 CSFs (R²=0.43, p=0.0046) (FIG. 5C), supporting the specificity and reliability of our 71A1 affinity purification. Here we also calculated the Aβ mass in ng from IPs and post-IPs by 4 antibodies from the 3 CSFs in FIG. 5A. We found that IP+post IP were not perfectly matched for the 4 different antibodies, unlike the brain soaking extracts, although they shared the same trend across the 3 individual CSF samples, a finding which precluded our using IP-ELISA further to quantify the oAβ accurately from biofluids samples.

The above result highlights the technical limitations of the Aβ IP-ELISA approach for quantifying the relative levels of 71A1-reactive Aβ species in large numbers of brain, CSF and potentially plasma samples. Therefore, we proceeded to develop sandwich immunoassays to more accurately quantify apparent Aβ oligomers in biofluids. In this context, we had previously quantified 1C22-reactive Aβ oligomers in human CSF, but 1C22 pulled down small amounts of CSF Aβ oligomers (FIG. 5A), forcing us to use CSF without dilution (neat) to get sufficient levels from all samples, even with the high-sensitivity Erenna Immunoassay System (Millipore). Taking advantage of the unique property (above) of 71A1 having a higher binding capability towards Aβ oligomers in CSF than in brain extracts (in direct contrast to 1C22), we designed a sandwich immunoassay using 71A1 as capture to better quantify oAβ levels in biofluids such as CSF and ultimately plasma.

### Example 4.4 the 71A1/3D6 Aβ Say Sensitively and Specifically Quantifies Aβ Oligomers

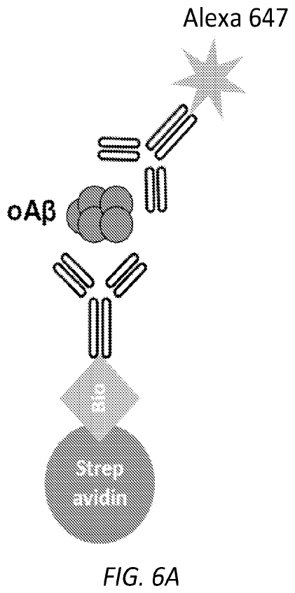

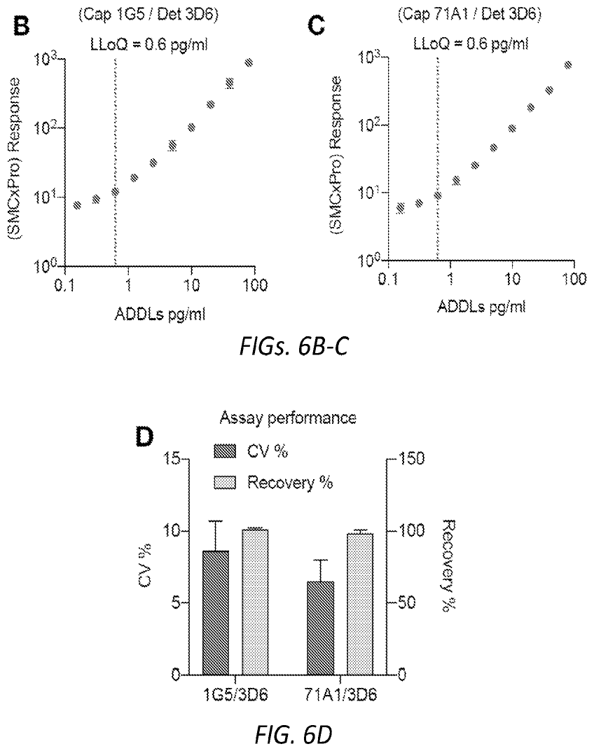

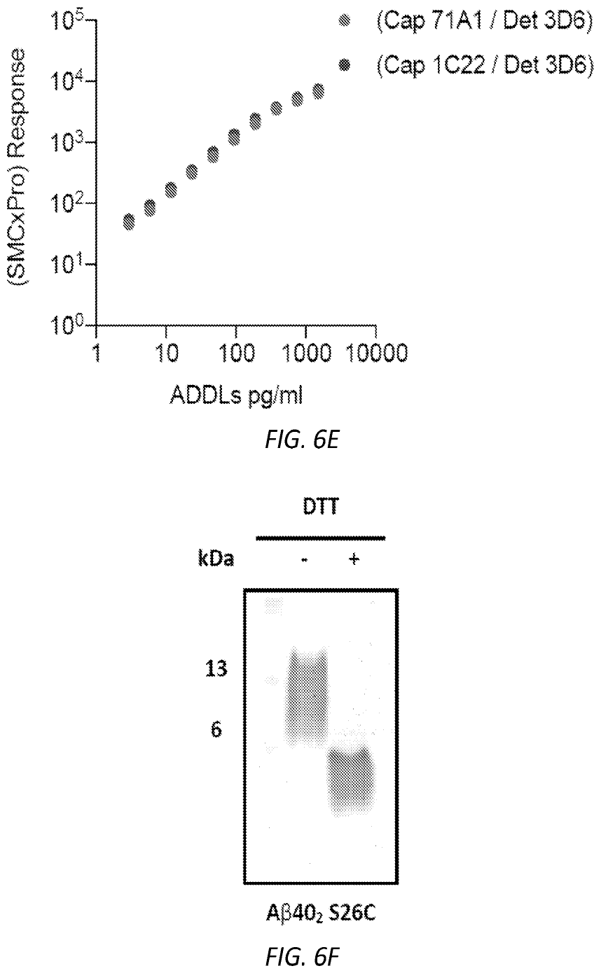

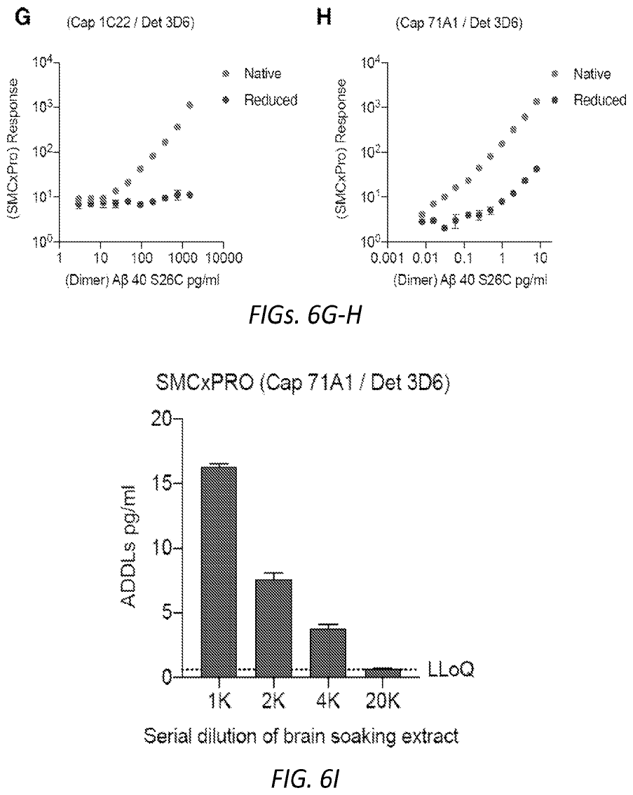

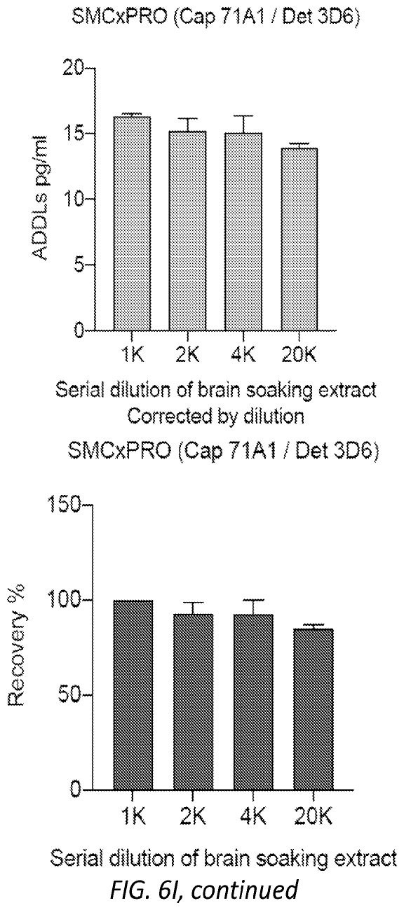

We developed sensitive immunoassays to quantify 1G5- and 71A1-reactive Aβ species for detection in human CSF and plasma. Similar to our established 1C22/3D6 oAβ sandwich ELISA on the Erenna (Millipore) platform [30, 31], we used biotinylated 1G5 or 71A1 as the capture antibody conjugated to magnetic streptavidin-coated beads, and 3D6 (labeled with Alexa-647 dye) as the detector antibody on the bead-based immunoassay platform SMCxPRO (an upgraded version of Erenna from Millipore) (FIG. 6A). As shown in FIGS. 6B-D, when we used a conventional calibrator of synthetic ADDLs [1], both 71A1/3D6 and 1G5/3D6 assays gave 1) a LLoQ of 0.6 pg/mL, the same as our previously developed 1C22/3D6 oligomer assay [30]; 2) recovery calculated between 80 & 120%; and 3) intra-assay CV well below 20% over the ADDL concentration range from 0.6 to 80 μg/mL, which readily covers the entire range of detectable analyte concentrations at appropriate dilution factors used in CSF and plasma (see below). As observed in our IP-ELISA assay in which 71A1 outperformed 1G5 in terms of pull-down efficacy and consistency (FIG. 5A), we focused on validating the 71A1/3D6 assay for its specificity towards oligomeric Aβ found in human biofluids.

To assess whether 71A1 preferentially recognizes oAβ over monomeric Aβ, we utilized a synthetic S26C Aβ40 dimer [45] covalently linked via a disulfide bond, which can be reversed by a reducing agent such as DTT into its monomeric form. Before we compared the new 71A1/3D6 and earlier 1C22/3D6 assays on this Aβ40 dimer, we first demonstrated that the two assays have the same sensitivity towards ADDLs (FIG. 6E). Both the 1C22/3D6 oAβ assay and the 71A1/3D6 assay showed a strong and significant binding preference for the non-reduced S26C Aβ40 dimer (confirmed by SDS-PAGE in FIG. 6F) over its reduced monomeric counterpart (FIGS. 6G-H). This result suggests that both 1C22 and 71A1 antibodies preferentially bind to Aβ with higher order structures, while binding monomeric Aβ more than 100-fold less. Moreover, the 71A1/3D6 assay showed much greater sensitivity toward the S26C Aβ40 dimer than did the 1C22/3D6 assay: 8 μg/mL S26C Aβ40 dimer in the 71A1/3D6 assay had similar signal as 1.5 ng/mL S26C Aβ40 dimer in the 1C22/3D6 assay (FIG. 6G vs. 6H; note difference in units on the abscissas), which may help explain the difference observed between 71A1 and 1C22 in terms of their respective binding preferences toward AD brain derived oAβ versus oAβ in CSF (compare FIGS. 3C and 5A).

Having assessed the sensitivity and specificity of the 71A1/3D6 assay on synthetic oligomeric vs. monomeric Aβ, we proceeded to test whether the 71A1/3D6 assay can readily and accurately measure oAβ from natural sources, such as human brain soaking extracts. We serially diluted a human brain soaking extract (tissue:soaking buffer=1:5 w/v) to 1-, 2-, 4- and 20-thousand-fold and measured the diluted samples with the 71A1/3D6 assay (FIG. 6I). We found that the signals from the diluted brain soaking extract samples were perfectly dilutable, with the average % recovery at 20,000-fold being 93.3% (FIG. 6I, bottom panel). This dilution linearity test of the 71A1/3D6 assay demonstrated its capacity to measure oAβ from natural biological materials over a wide concentration range.

### Example 4.5 the 71A1/3D6 Aβ Say Recognizes High Molecular Weight Aβ Oligomers in Brain

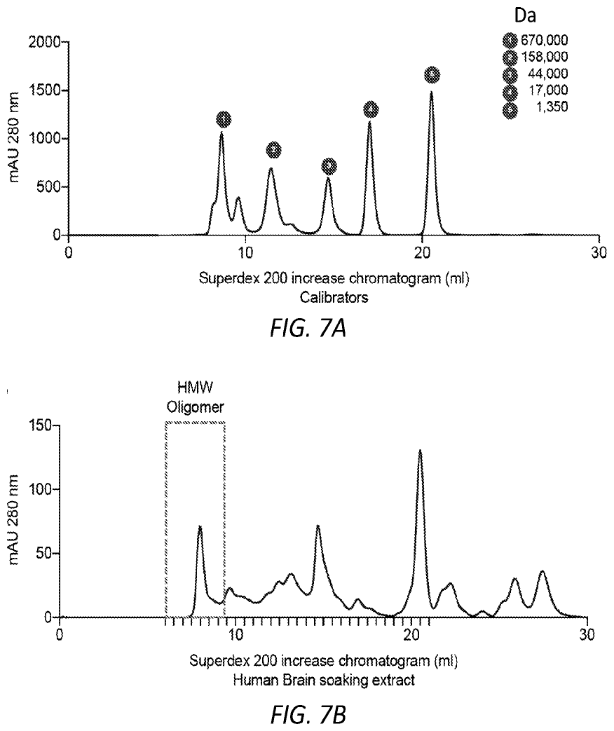

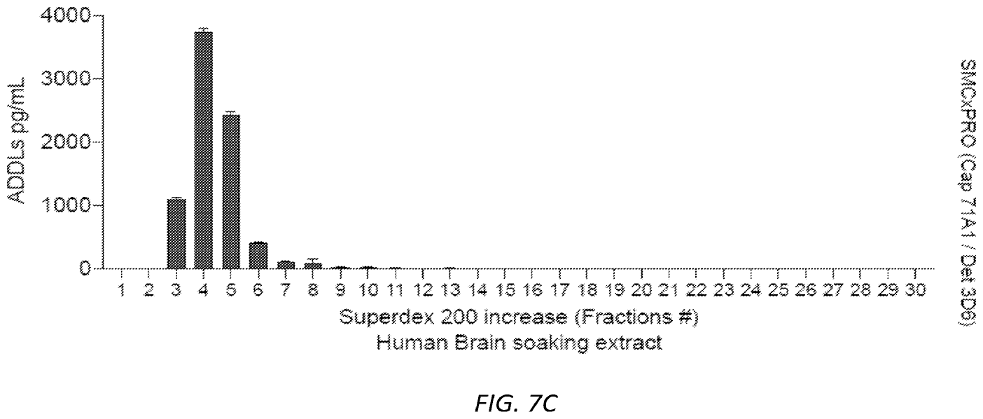

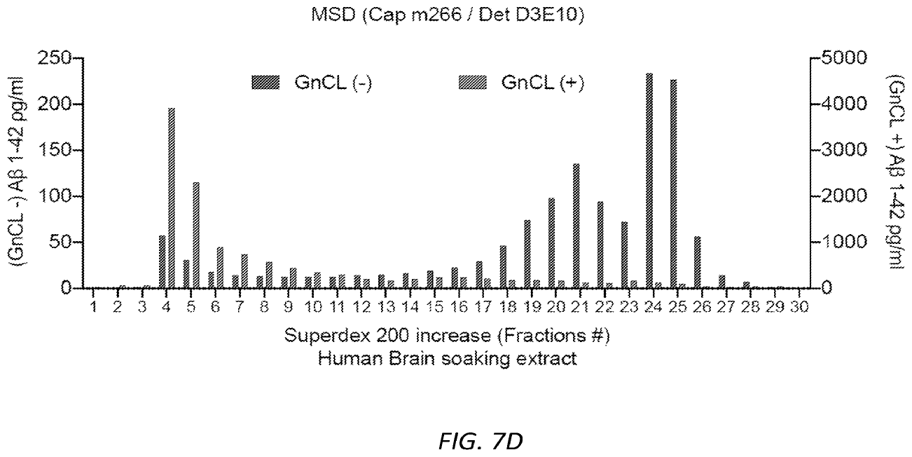

Next, we explored what kind of natural oAβ species are detected by the 71A1/3D6 immunoassay. Using non-denaturing size exclusion chromatography (SEC), we fractionated human AD brain soaking extracts on a Superdex 200 Increase high resolution column, as calibrated by molecular weight (MW) standards (FIG. 7A). We performed the 71A1/3D6 assay on 30 SEC fractions starting from the void volume of the column (UV280 chromatogram of the fractions in FIG. 7B). The 71A1/3D6 signal (measured in pg/mL units of the ADDL calibrator) peaked at fractions #3-6, which are within the void volume range, indicating that the estimated analyte size is at least 670 kDa (FIG. 7C). To confirm that this 71A1/3D6 signal in fractions #3-6 indeed represents Aβ, we used 8M GnCl to denature the 30 fractions and then measured them with a well-established monomeric Aβ42 immunoassay (capture: m266, detector: D3E10, on the MSD platform) [11, 50]. We found that the GnCl-released monomeric Aβ species peaked at the same fractions #3-6 as the 71A1/3D6 assay (compare FIG. 7C to FIG. 7D [GnCl (+)=lighter grey bars, right side in each pair bars]). With the same Aβ monomer assay, we found that the natively monomeric species (FIG. 7D: GnCl (−)=darker grey bars, left side in each pair) primarily eluted into fractions #19-26. Together, the data suggest that the abundant 71A1/3D6 signals observed in fractions #3-6 of AD brain soaking extracts represent higher order, high MW soluble Aβ oligomers which can be denatured by GnCl into Aβ monomers, and the assay detects monomers very poorly, similar to the pattern that the 1C22/3D6 oAβ assay detected previously [30, 53].

### Example 4.5 the 71A1/3D6 Aβ Say Quantifies Low Molecular Weight Aβ Oligomers in Human CSF

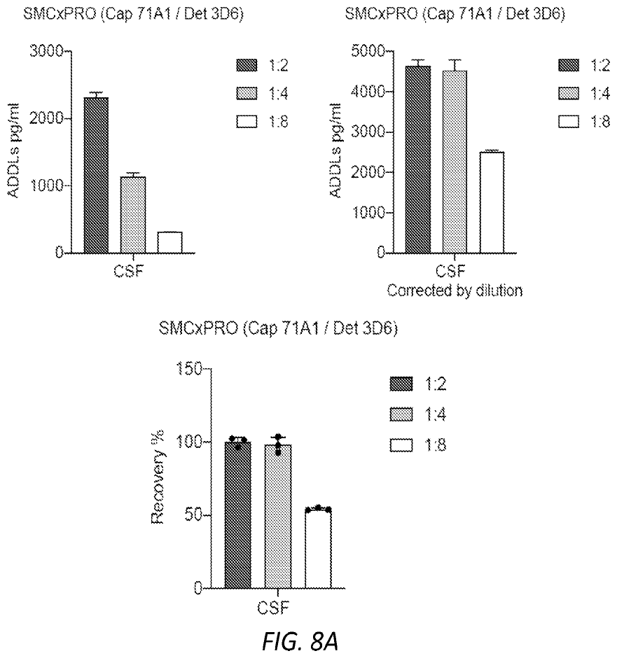

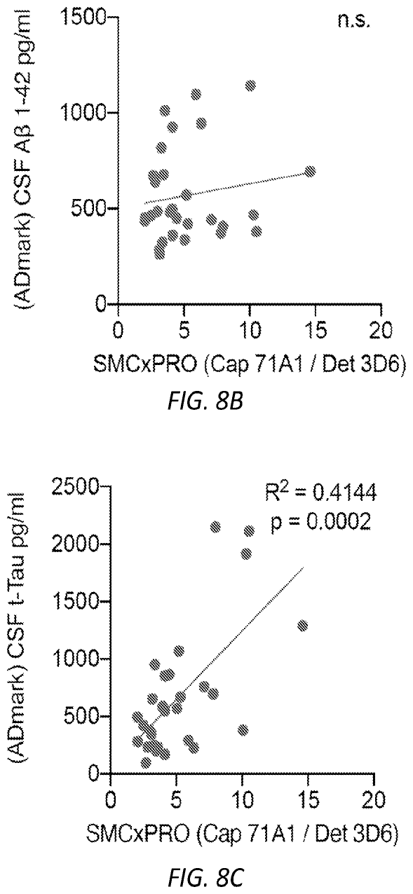

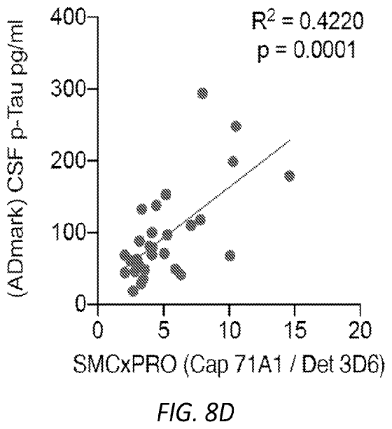

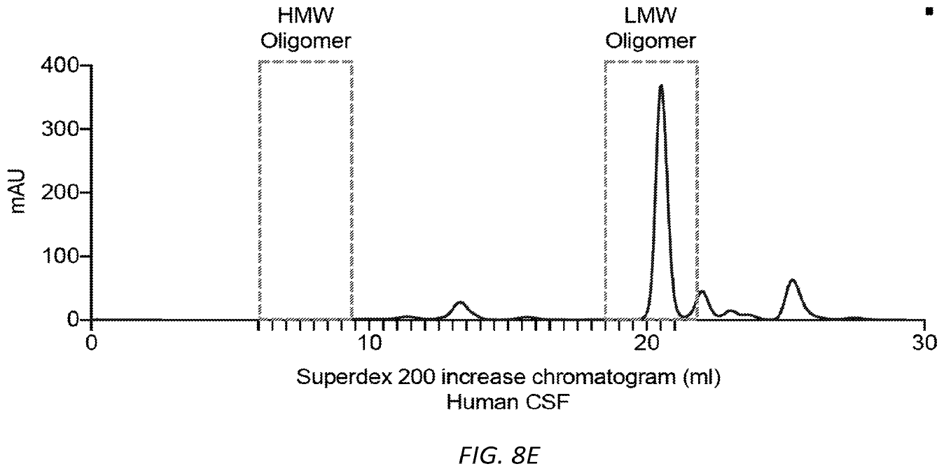

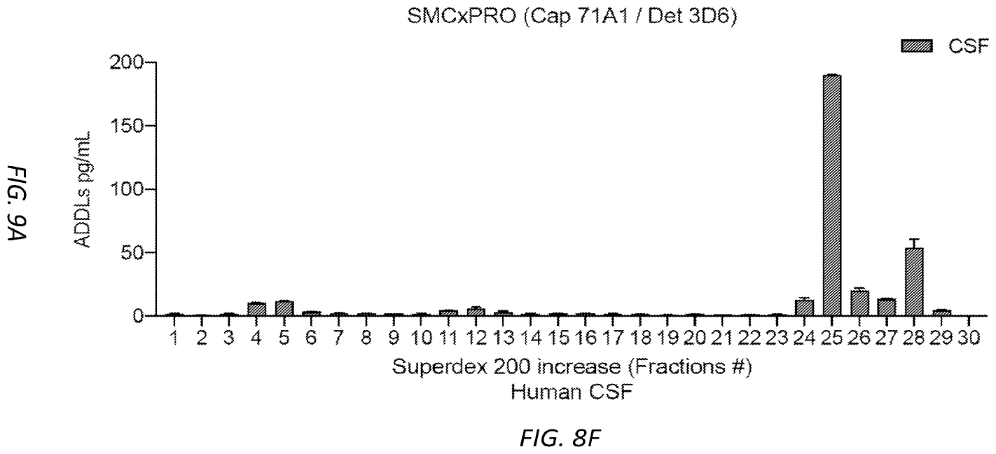

To validate the 71A1/3D6 assay on human CSF we performed dilution linearity testing, similar to the steps used on brain soaking extracts (FIG. 8A). We observed an average recovery of 98.94% for 1:2 and 1:4 dilutions, indicating that the measurements were accurate and stable in that range, but the dilution recovery was suboptimal at 1:8 dilution (FIG. 8A) for unknown reasons. We proceeded to measure CSF from a cohort of 36 subjects with varied neurological diagnoses collected in our Memory Disorders Clinic (Brigham and Women's Hospital) using the 71A1/3D6 assay (Table 1: patient demographic and diagnostic information). All 36 samples were quantified by the 71A1/3D6 assay, with the average level being 5.47 ng/mL and a range of 1.75 to 20.38 ng/mL (FIG. 8B-D). We asked whether these CSF 71A1/3D6 levels correlated with other biomarkers of AD pathology in CSF, as measured by the widely used commercial ADmark clinical assay (Athena Diagnostics). As ADmark data was available for 29 out of 36 samples, the correlations were performed on these 29 samples. There was no statistically significant correlation between 71A1/3D6 oAβ signals and ADmark Aβ1-42 monomer levels in these CSFs (FIG. 8B), but there were strongly significant correlations between 71A1/3D6 signals and both the ADmark total tau (R²=0.41, p=0.0002) and phosho-tau (pT181) (R²=0.42, p=0.0001; Pearson correlation coefficients) (FIG. 8C-D). Similar to our analysis of the brain soaking extract SEC fractions (FIG. 7A-D), we performed the 71A1/3D6 assay on 30 SEC fractions of human CSF starting from the void volume of the column (UV280 chromatogram shown in FIG. 8E). The 71A1/3D6 signal (measured in pg/mL units of the ADDL calibrator) had two peaks: a smaller one at #3-6 and a larger one at #24-28 (FIG. 8F), which was a different distribution than the brain soaking extract (FIG. 7C). These data together demonstrate that the 71A1/3D6 assay can quantify the low molecular weight Aβ oligomers found in human CSF in addition to the high molecular weight oligomers found in the brain soaking extract.

### Example 4. 6 the 71A1/3D6 Assay Quantifies Aβ Oligomers in Human Plasma

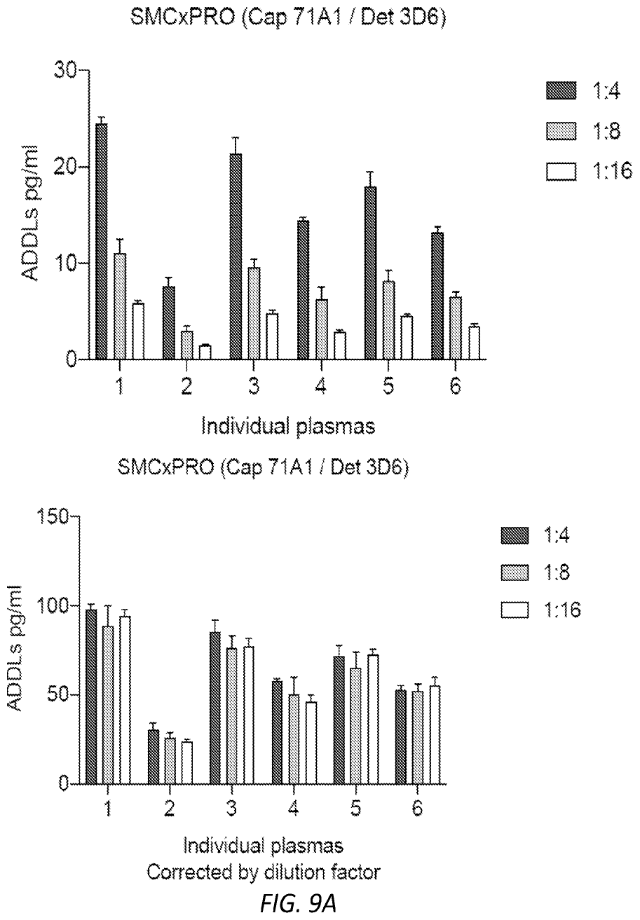

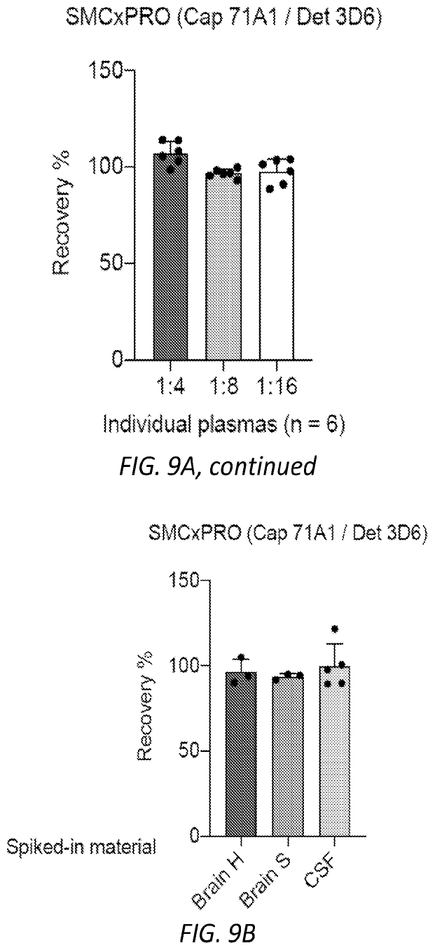

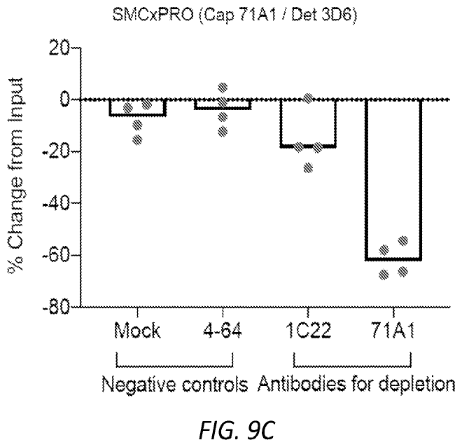

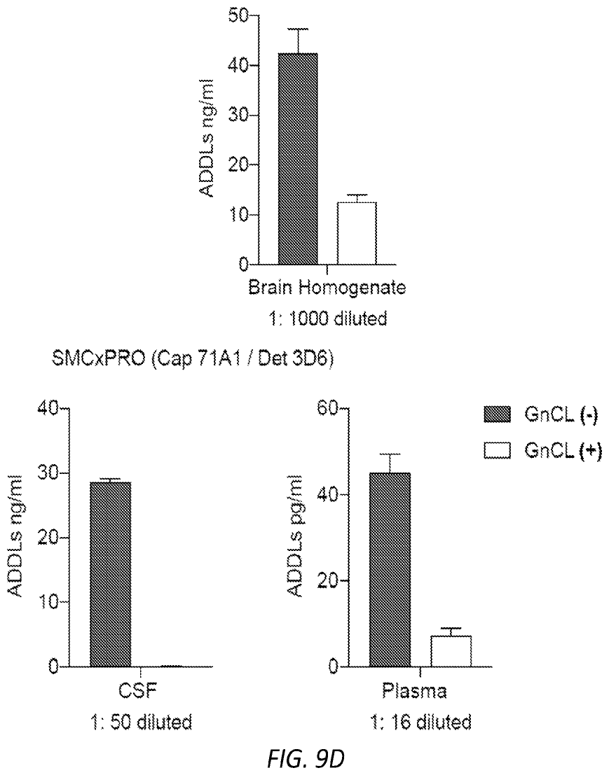

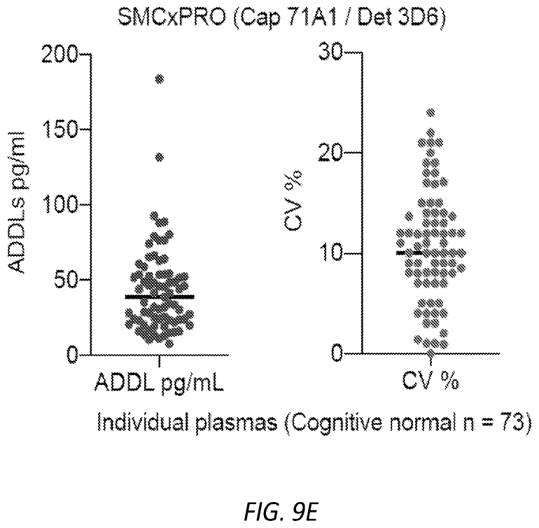

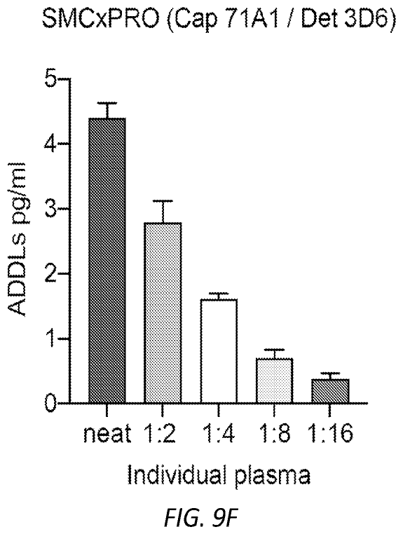

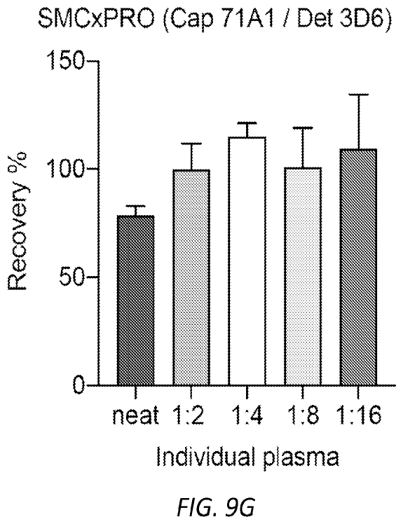

CSF is far less attractive for biomarker development because its collection is viewed as too invasive by many people and is relatively cumbersome and expensive. Moreover, serial lumbar puncture measurements are very rarely done. In contrast, blood collection is routinely performed, minimally invasive and inexpensive. We therefore sought to establish the accuracy of the 71A1/3D6 assay in plasma, a highly complex matrix posing technical challenges for accurate quantification by immunoassay. We tested for the degree of matrix interference in the plasma via 1) plasma dilution and recovery; 2) analyte spike-and-recovery; and 3) immunodepletion. Relative to a 4-fold dilution, the 71A1/3D6 assay showed near 100% dilution recovery (mean of 96.6%) at up to 16-fold dilution of each of six individual plasmas (from the Mayo Clinic Alzheimer's Disease Research Center) (FIG. 9A). We also observed a matrix effect in plasma on this assay only if using neat plasma (without dilution) (FIGS. 9F-G). Therefore, we chose to use the 1:8 dilution for the following experiments. We conducted analyte spike-and-recovery experiments on 8-fold diluted plasma samples using three different sources of natural oAβ from AD subjects, brain homogenate, brain soaking extract and CSF, which gave excellent mean recovery values of 97.5%, 92.9%, and 99.7%, respectively (FIG. 9B). Then, we attempted immunodepletion experiments on 8-fold diluted individual plasmas using biotinylated antibodies and streptavidin-coated plates. 71A1 immunodepleted an average of 61.5% of the 71A1/3D6 signal whereas 1C22 immuno-depleted only 15.6% of the 71A1/3D6 signal (FIG. 9C), demonstrating the specificity of the 71A1/3D6 assay for oAβ detection in plasma. Further, we examined the specificity of the 71A1/3D6 assay toward Aβ oligomers vs. monomers in brain homogenate, CSF and plasma. We measured these samples with or without 8M GnCl oligomer-denaturing treatment and found that GnCl decreased the 71A1/3D6 signal from brain homogenate by >70% and from plasma by >84%, while abolishing the signal from CSF (>99%) (FIG. 9D). All the samples were extensively diluted before assaying to eliminate the negative effects of GnCl on the immunoassay (final concentration of GnCl <0.25 M).

In summary, data from plasma dilution-recovery and spike-and-recovery experiments with three different natural sources of oAβ from AD subjects all demonstrated no significant matrix interference in the plasma 71A1/3D6 assay. Further, the immunodepletion experiments highlight the ability of 71A1 to bind its target in individual human plasmas. We therefore performed the 71A1/3D6 immunoassay on 8-fold diluted plasma samples on a cohort of 73 cognitively normal individuals (Mayo Clinic Alzheimer's Disease Research Center). The mean dilution-adjusted concentration based on the synthetic oAβ (ADDL) standard curve was 43.34±29.09 pg/mL (FIG. 9E, left). The CVs of these quadruplicate plasma samples were ≤20% except for five subjects (FIG. 9E, right), demonstrating the consistency of the plasma assay.

### Example 5. An Oligomer-Directed Aβ Antibody Decreases Synaptotoxic Oligomeric Aβ in Brain and Improves Behavioral Abnormality in Humanized APP Knock-In Mice

There is strong evidence that soluble oligomers of amyloid beta protein (oAβ) help initiate the pathogenic cascade of Alzheimer's disease (AD), which suggests therapeutic strategies targeting oAβ over monomeric or fibrillar Aβ. A new antibody, 71A1, is ˜100-fold more sensitive for oAβ than synthetic monomers. The material that 71A1 specifically immunoprecipitates from AD soluble brain extracts impairs synaptic function as much as does the full extract. In accord, pre-incubating brain extracts with 71A1 neutralizes its synaptotoxicity. 71A1 has potentially unique activities against disease-relevant oAβ, making it a novel candidate for treating AD.

### Methods

15 mg/kg 71A1 or anti-KLH (negative control) was administered i.p. to 45 humanized APP knock-in mice (APPNLGF/NLGF) weekly from 8 to 20 weeks of age. Cognition was assessed by spontaneous alternation (Y-maze). Brains were harvested at age 21-wk for biochemical, electrophysiological and immunohistochemical analyses. Diffusible (“soaking”) extracts of the brains were prepared to measure monomeric and oligomeric Aβ using homebrew ultra-sensitive assays. Aliquots of the same soaking extracts were used to treat wild-type mouse hippocampal slices and measure long-term potentiation (LTP) to assess synaptotoxicity in the APP^{NLGF/NLGF} brains post-treatment.

### Results

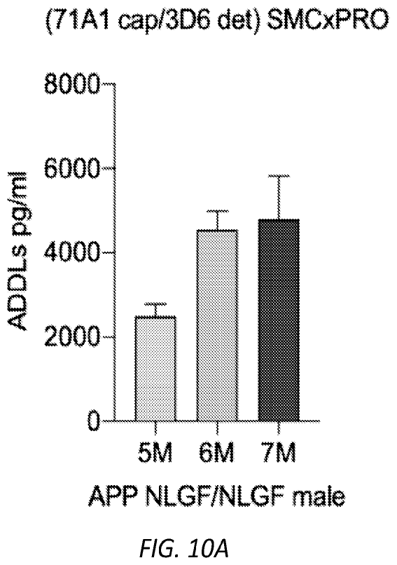

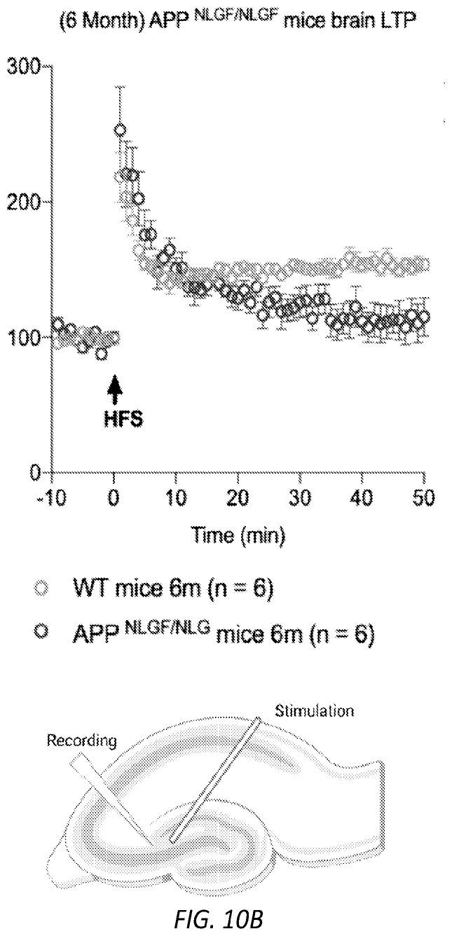

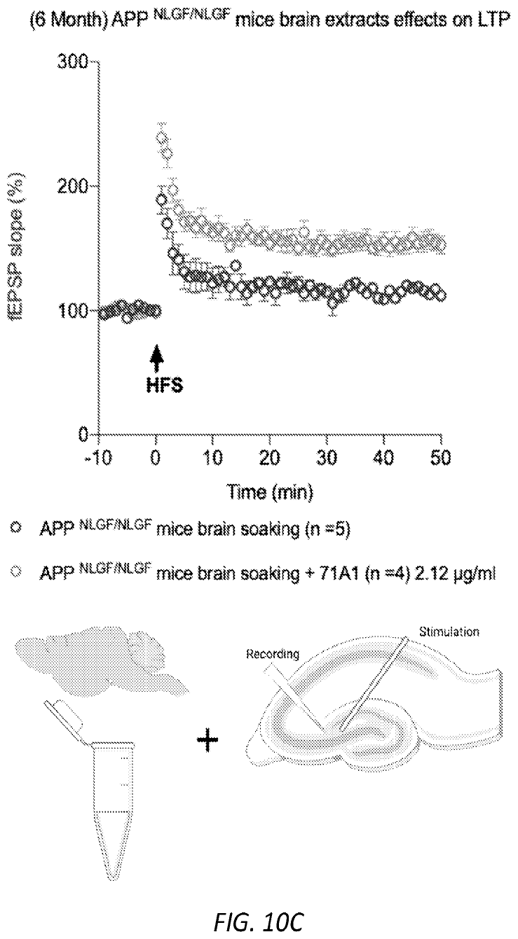

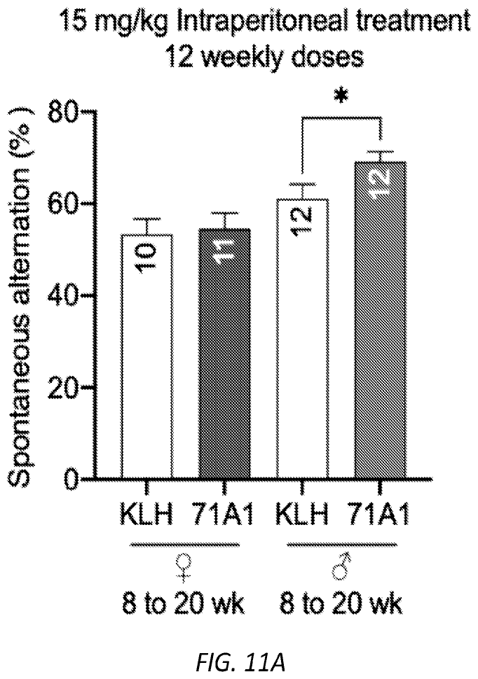

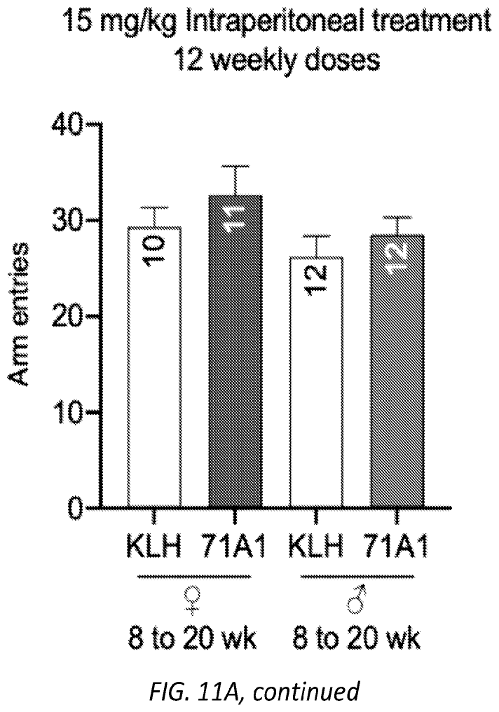

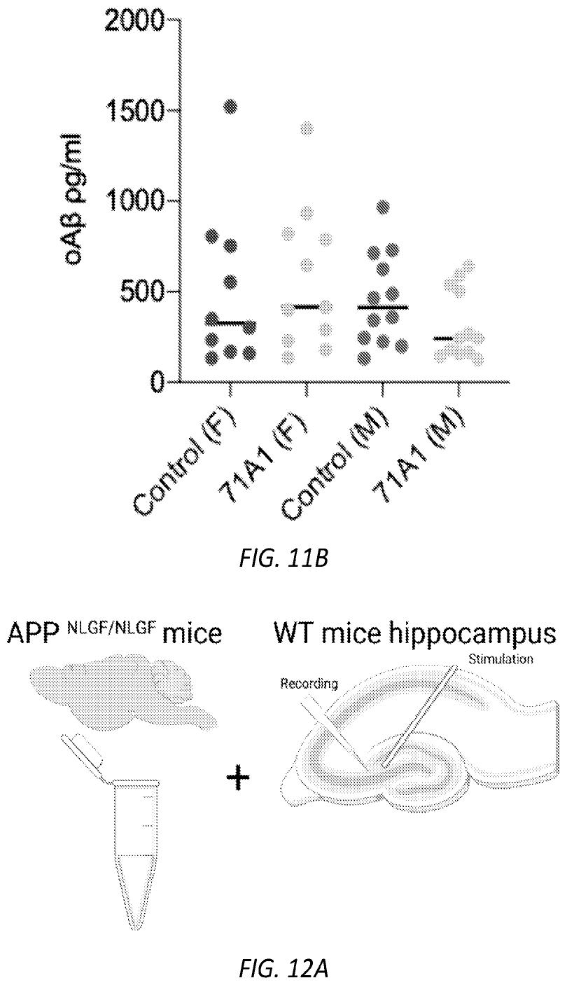

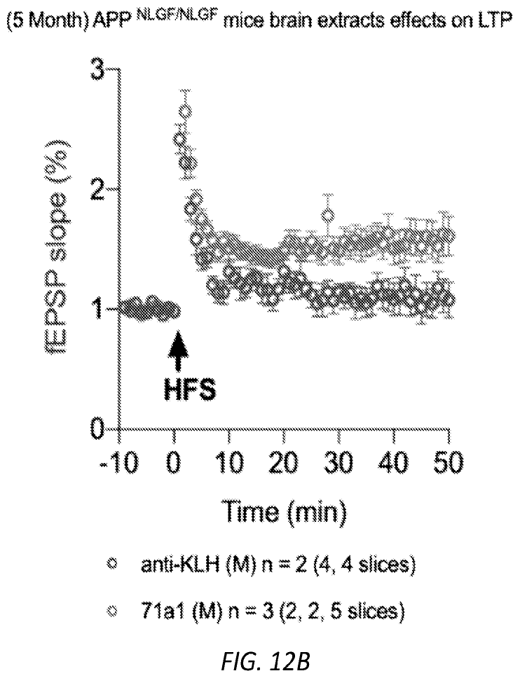

oAβ from brains of 6M old APP^{NLGF/NLGF} impaired the LTP of wild-type mice, which is rescued by treatment with 71A1 (see FIGS. 10A-C). As shown in FIG. 11A, 71A1 treatment significantly improved Y-maze performance of male mice but not female compared to control antibody treatment in. At that time point, 71A1 treatment decreased oAβ amount in brain of male mice (a trend without statistical significance, FIG. 11B). In addition, 71A1 treatment reduced the synaptotoxicity of APP NLGF male mouse brain, reflected by failure to inhibit LTP of wild-type mice hippocampus (see FIGS. 12A-B).

These results demonstrate 71A1 is an oligomer-preferring monoclonal antibody as a therapeutic candidate for AD, decreasing brain oAβ and oAβ-induced synaptotoxicity. We believe this provides the first proof-of-concept mouse trial using an antibody that has been carefully characterized as oAβ-preferring. We observed 1) a correlation between oAβ concentration and its synaptotoxicity; and 2) that 71A1 ameliorated aspects of cognitive impairment in APPNLGF/NLGF mice by targeting oAβ.

### Example 6. Identification of Peptide Mimetics with Binding to 71A1 Variable Regions

A New England Biolabs (NEB) Ph.D.-12™ Phage Display Peptide Library Kit (instructions attached) was used to isolate amino acid residue peptide ligands (referred to herein as peptide mimetics) that bound to immobilized Mab 71A1. The New England Biolabs Ph.D.-12™ Phage Display Peptide Library Kit is a combinatorial library of random, 12 amino acid peptides (12-mers) fused to a minor coat protein (pIII) of the M13 bacteriophage. The peptide was expressed as a fusion with a coat protein that produces a display of the fused protein on the surface of the bacteriophage. The library consists of 2.7×109 electroporated sequences, amplified once to yield approximately 55 copies of each sequence in 10 μl of the supplied phage.

Immobilized 71A1 antibody was used to isolate the 12 amino acid peptides, shown in FIG. 13, that bound to the variable region. Thermo Scientific™ AminoLink Plus Coupling Resin was used to immobilize Mab 71A1. Negative control particles were made using monoclonal antibody 3C7 with specificity for an irrelevant peptide sequence. The Thermo Scientific™ AminoLink Plus Coupling Resin Protocol is provided that was used to produce the 71A1 particles panned with the NEB library. Briefly, 2 mL aminolink particle slurry was washed twice with 10 mL, 0.1 M sodium bicarbonate buffer pH 10 in a 15 mL conical tube by centrifugation for 10 minutes at 2000×g. 10 mgs of 71A1 antibody in this binding buffer was then added to the particles transferred to a 2 mL eppendorph tube and rotated at room temperature for 2 hours. The slurry was then added to a Millipore, Ultrafree-MC centrifugal filter unit and the resin washed 3× in 2 mL, 0.1M sodium phosphate, 0.15M NaCl, pH 7.2 binding buffer. Spun particles were then mixed with 2 mL pH 7.2 buffer and 40 μl of 50 mM Sodium Cyanoborohydride Solution and rotated for 4 hours. The aminoLink gel contains aldehyde functional groups that spontaneously react with primary amines on the antibody. The Schiff base bonds that form are then reduced to stable secondary amine bonds in the presence of the mild reducing agent, sodium cyanoborohydride. Particles were then washed twice with 2 mL 1M Tris HCL, pH 7.4, resuspended in 2 mL of this same buffer and 40 μl Sodium cyanoborohydride to block unbound sites on the particles, and rotated for 30 minutes. Particles washed 5 times with 1M sodium chloride were suspended in 1 mL PBS.

The library (10 μl) was first mixed with the isotype matched, variable region irrelevant, immobilized 3C7 antibody particles (200 μl added to a Milipore, Ultrafree-MC Centrifugal filter unit) to remove non-specifically bound bacteriophage. The library was removed from these control particles by spinning a spun at 2000×g, and then mixed with immobilized 71A1 antibody (200 μl) onto a new Milipore, Ultrafree-MC Centrifugal filter unit and unbound phage subsequently washed away by centrifugation at 2000×g. The particles were subsequently washed with 1 mL PBS and the procedure was repeated several times. Specifically bound phage were eluted from the immobilized antibody using 100 μl, 0.1 M Glycine-HCl, pH 2.0. The bacteriophage were amplified by infection and growth of liquid culture E. coli bacteria as described in the protocol supplied with the kit and included here. Isolated, amplified bacteriophage were then isolated from the bacterial supernatant using PEG/NaCl (20% (w/v) polyethylene glycol-8000, 2.5 M NaCl) precipitation. The isolated phage were amplified and taken through additional binding/amplification cycles to enrich the pool of phage for binding sequences to the antibody variable region. After 3-4 rounds, individual clones were characterized by DNA sequencing (AIBioTech—American International Biotechnology Services, Richmond, VA), and subsequent amino acid predictions (FIG. 13) made from the isolated DNA sequences.

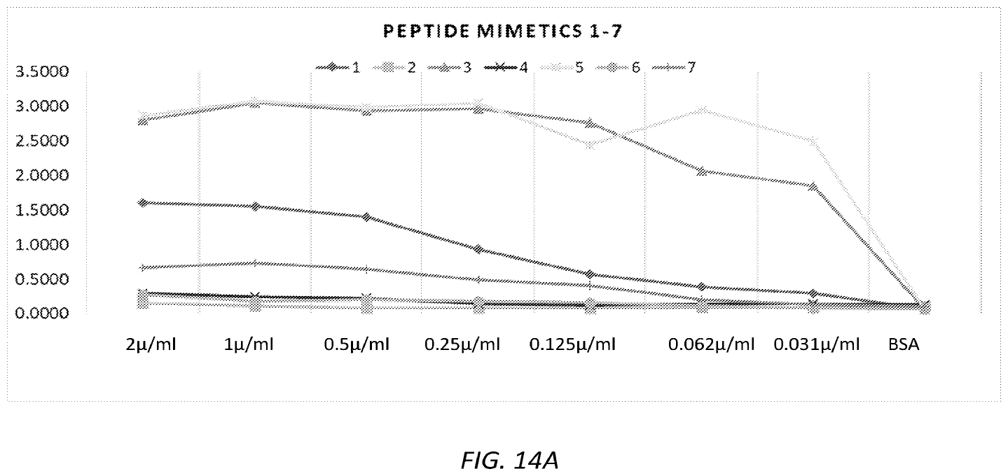

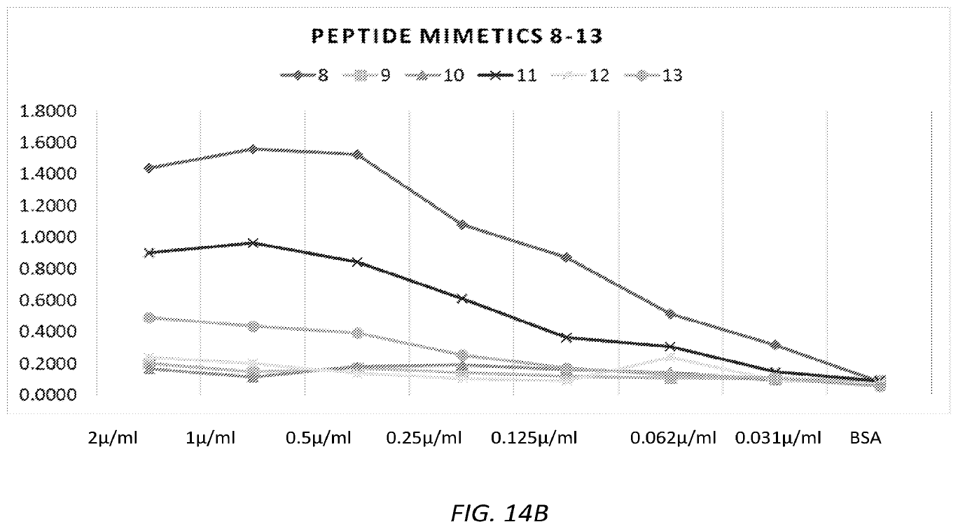

A standard, indirect enzyme linked immunosorbent assay (ELISA) with purified monoclonal antibody 71A1 was used to assay for binding to peptide mimetics 1-13 (sequences in FIG. 13) conjugated to maleimide-activated micro titer plates at 10 μg/mL in 0.1M Sodium bicarbonate buffer pH10 and blocked with PBS and 2% BSA. After one hour incubation, the wells were washed and then 100 ul of a secondary, Horseradish peroxidase (HRPO)-conjugated goat anti-mouse antibody was added and the plates, incubated for an additional hour before addition of TMB (3,3′,5,5′-Tetramethylbenzidine) substrate. Absorbance was measured in a spectrophotometer at 650 nm. The results, shown in FIGS. 14A-B, demonstrate that most, but not all, of the peptide mimetics isolated by phage display bind to Monoclonal Antibody 71A1; in this assay, peptides 1, 3, 5, 7, 8, 11, and 13 showed the most binding, with peptides 5, 8, and 7 being the best.

### REFERENCES

1. Lambert M P, Barlow A K, Chromy B A, Edwards C, Freed R, Liosatos M et al. Diffusible, nonfibrillar ligands derived from Abeta1-42 are potent central nervous system neurotoxins. Proc Natl Acad Sci USA 1998; 95: 6448-6453.
2. Walsh D M, Klyubin I, Fadeeva J V, Cullen W K, Anwyl R, Wolfe M S et al. Naturally secreted oligomers of amyloid beta protein potently inhibit hippocampal long-term potentiation in vivo. Nature 2002; 416: 535-539.
3. Lacor P N, Buniel M C, Furlow P W, Clemente A S, Velasco P T, Wood M et al. Aβ oligomer-induced aberrations in synapse composition, shape, and density provide a molecular basis for loss of connectivity in Alzheimer's disease. Journal of Neuroscience 2007; 27: 796-807.
4. Koffie R M, Meyer-Luehmann M, Hashimoto T, Adams K W, Mielke M L, Garcia-Alloza M et al. Oligomeric amyloid beta associates with postsynaptic densities and correlates with excitatory synapse loss near senile plaques. Proc Natl Acad Sci USA 2009; 106: 4012-4017.
5. Sondag C M, Dhawan G, Combs C K. Beta amyloid oligomers and fibrils stimulate differential activation of primary microglia. Journal of neuroinflammation 2009; 6: 1-13.
6. Dhawan G, Floden A M, Combs C K. Amyloid-β oligomers stimulate microglia through a tyrosine kinase dependent mechanism. Neurobiology of aging 2012; 33: 2247-2261.
7. Xu H, Rajsombath M M, Weikop P, Selkoe D J. Enriched environment enhances β-adrenergic signaling to prevent microglia inflammation by amyloid-β. EMBO molecular medicine 2018; 10: e8931.
8. Lue L F, Kuo Y M, Roher A E, Brachova L, Shen Y, Sue L et al. Soluble amyloid beta peptide concentration as a predictor of synaptic change in Alzheimer's disease. Am J Pathol 1999; 155: 853-862.
9. McLean C A, Cherny R A, Fraser F W, Fuller S J, Smith M J, Beyreuther K et al. Soluble pool of Abeta amyloid as a determinant of severity of neurodegeneration in Alzheimer's disease. Ann Neurol 1999; 46: 860-866.
10. Zott B, Simon M M, Hong W, Unger F, Chen-Engerer H J, Frosch M P et al. A vicious cycle of β amyloid-dependent neuronal hyperactivation. Science 2019; 365: 559-565.
11. Hong W, Wang Z, Liu W, O'Malley T T, Jin M, Willem M et al. Diffusible, highly bioactive oligomers represent a critical minority of soluble Aβ in Alzheimer's disease brain. Acta Neuropathol 2018; 136: 19-40.
12. Li S, Hong S, Shepardson N E, Walsh D M, Shankar G M, Selkoe D. Soluble oligomers of amyloid Beta protein facilitate hippocampal long-term depression by disrupting neuronal glutamate uptake. Neuron 2009; 62: 788-801.
13. Li S, Jin M, Liu L, Dang Y, Ostaszewski B L, Selkoe D J. Decoding the synaptic dysfunction of bioactive human A D brain soluble Aβ to inspire novel therapeutic avenues for Alzheimer's disease. Acta Neuropathol Commun 2018; 6: 121.
14. Shankar G M, Li S, Mehta T H, Garcia-Munoz A, Shepardson N E, Smith I et al. Amyloid-beta protein dimers isolated directly from Alzheimer's brains impair synaptic plasticity and memory. Nat Med 2008; 14: 837-842.
15. Esparza T J, Zhao H, Cirrito J R, Cairns N J, Bateman R J, Holtzman D M et al. Amyloid-β oligomerization in Alzheimer dementia versus high-pathology controls. Ann Neurol 2013; 73: 104-119.
16. Wong C W, Quaranta V, Glenner G G. Neuritic plaques and cerebrovascular amyloid in Alzheimer disease are antigenically related. Proc Natl Acad Sci USA 1985; 82: 8729-8732.
17. Selkoe D J, Abraham C R, Podlisny M B, Duffy L K. Isolation of low-molecular-weight proteins from amyloid plaque fibers in Alzheimer's disease. J Neurochem 1986; 46: 1820-1834.
18. Bard F, Cannon C, Barbour R, Burke R L, Games D, Grajeda H et al. Peripherally administered antibodies against amyloid beta-peptide enter the central nervous system and reduce pathology in a mouse model of Alzheimer disease. Nat Med 2000; 6: 916-919.
19. Lambert M P, Viola K L, Chromy B A, Chang L, Morgan T E, Yu J et al. Vaccination with soluble Aβ oligomers generates toxicity-neutralizing antibodies. Journal of neurochemistry 2001; 79: 595-605.
20. O'Nuallain B, Wetzel R. Conformational Abs recognizing a generic amyloid fibril epitope. Proc Natl Acad Sci USA 2002; 99: 1485-1490.
21. Lambert M P, Velasco P T, Chang L, Viola K L, Fernandez S, Lacor P N et al. Monoclonal antibodies that target pathological assemblies of Abeta. J Neurochem 2007; 100: 23-35.
22. O'Nuallain B, Klyubin I, Mc Donald J M, Foster J S, Welzel A, Barry A et al. A monoclonal antibody against synthetic Aβ dimer assemblies neutralizes brain-derived synaptic plasticity-disrupting Aβ. Journal of neurochemistry 2011; 119: 189-201.
23. Morgado I, Wieligmann K, Bereza M, Rönicke R, Meinhardt K, Annamalai K et al. Molecular basis of β-amyloid oligomer recognition with a conformational antibody fragment. Proceedings of the National Academy of Sciences 2012; 109: 12503-12508.
24. Savage M J, Kalinina J, Wolfe A, Tugusheva K, Korn R, Cash-Mason T et al. A sensitive aβ oligomer assay discriminates Alzheimer's and aged control cerebrospinal fluid. Journal of Neuroscience 2014; 34: 2884-2897.
25. Phay M, Welzel A T, Williams A D, McWilliams-Koeppen H P, Blinder V, O'Malley T T et al. IgG Conformer's Binding to Amyloidogenic Aggregates. PLoS One 2015; 10: e0137344.
26. Mably A J, Liu W, Mc Donald J M, Dodart J C, Bard F, Lemere C A et al. Anti-Aβ antibodies incapable of reducing cerebral Aβ oligomers fail to attenuate spatial reference memory deficits in J20 mice. Neurobiol Dis 2015; 82: 372-384.
27. Murakami K, Tokuda M, Suzuki T, Irie Y, Hanaki M, Izuo N et al. Monoclonal antibody with conformational specificity for a toxic conformer of amyloid β42 and its application toward the Alzheimer's disease diagnosis. Sci Rep 2016; 6: 29038.
28. Ochiishi T, Itakura A, Liu L, Akatsu H, Kohno H, Nishimura M et al. Immunohistochemical detection of the delayed formation of nonfibrillar large amyloid-β aggregates. Genes Cells 2016; 21: 200-211.
29. Gibbs E, Silverman J M, Zhao B, Peng X, Wang J, Wellington C L et al. A rationally designed humanized antibody selective for amyloid beta oligomers in Alzheimer's disease. Scientific reports 2019; 9: 1-14.
30. Yang T, T O'Malley T, Kanmert D, Jerecic J, Zieske L R, Zetterberg H et al. A highly sensitive novel immunoassay specifically detects low levels of soluble Aβ oligomers in human cerebrospinal fluid. Alzheimer's research & therapy 2015; 7:1-16.
31. Yang T, Dang Y, Ostaszewski B, Mengel D, Steffen V, Rabe C et al. Target engagement in an Alzheimer trial: Crenezumab lowers amyloid β oligomers in cerebrospinal fluid. Annals of neurology 2019; 86: 215-224.
32. Liu L, Stern A, Dang Y, Ostaszewski B, Chhatwal J P, Selkoe D J. Predicting development of A D clinical symptoms and their progression through a collection of novel plasma Aβ immunoassays: Biomarkers (non-neuroimaging): Blood based biomarkers. Alzheimer's & Dementia 2020; 16: e043670.
33. Nakamura A, Kaneko N, Villemagne V L, Kato T, Doecke J, Doré V et al. High performance plasma amyloid-β biomarkers for Alzheimer's disease. Nature 2018; 554: 249-254.
34. Schindler S E, Bollinger J G, Ovod V, Mawuenyega K G, Li Y, Gordon B A et al. High-precision plasma β-amyloid 42/40 predicts current and future brain amyloidosis. Neurology 2019; 93: e1647-e1659.
35. Chen Z, Mengel D, Keshavan A, Rissman R A, Billinton A, Perkinton M et al. Learnings about the complexity of extracellular tau aid development of a blood-based screen for Alzheimer's disease. Alzheimer's & Dementia 2019; 15: 487-496.
36. Chhatwal J P, Schultz A P, Dang Y, Ostaszewski B, Liu L, Yang H-S et al. Plasma N-terminal tau fragment levels predict future cognitive decline and neurodegeneration in healthy elderly individuals. Nature Communications 2020; 11: 6024.
37. Mielke M M, Hagen C E, Xu J, Chai X, Vemuri P, Lowe V J et al. Plasma phospho-tau181 increases with Alzheimer's disease clinical severity and is associated with tau- and amyloid-positron emission tomography. Alzheimers Dement 2018; 14: 989-997.
38. Karikari T K, Pascoal T A, Ashton N J, Janelidze S, Benedet A L, Rodriguez J L et al. Blood phosphorylated tau 181 as a biomarker for Alzheimer's disease: a diagnostic performance and prediction modelling study using data from four prospective cohorts. The Lancet Neurology 2020; 19: 422-433.
39. Janelidze S, Stomrud E, Smith R, Palmqvist S, Mattsson N, Airey D C et al. Cerebrospinal fluid p-tau217 performs better than p-tau181 as a biomarker of Alzheimer's disease. Nat Commun 2020; 11: 1683.
40. Barthelemy N R, Bateman R J, Hirtz C, Marin P, Becher F, Sato C et al. Cerebrospinal fluid phospho-tau T217 outperforms T181 as a biomarker for the differential diagnosis of Alzheimer's disease and PET amyloid-positive patient identification. Alzheimers Res Ther 2020; 12: 26.
41. Thijssen E H, La Joie R, Wolf A, Strom A, Wang P, Iaccarino L et al. Diagnostic value of plasma phosphorylated tau181 in Alzheimer's disease and frontotemporal lobar degeneration. Nat Med 2020; 26: 387-397.
42. Palmqvist S, Janelidze S, Quiroz Y T, Zetterberg H, Lopera F, Stomrud E et al. Discriminative accuracy of plasma phospho-tau217 for Alzheimer disease vs other neurodegenerative disorders. Jama 2020; 324: 772-781.
43. Janelidze S, Mattsson N, Palmqvist S, Smith R, Beach T G, Serrano G E et al. Plasma P-tau181 in Alzheimer's disease: relationship to other biomarkers, differential diagnosis, neuropathology and longitudinal progression to Alzheimer's dementia. Nat Med 2020; 26: 379-386.
44. van Helmond Z, Heesom K, Love S. Characterisation of two antibodies to oligomeric Abeta and their use in ELISAs on human brain tissue homogenates. J Neurosci Methods 2009; 176: 206-212.
45. O'Nuallain B, Freir D B, Nicoll A J, Risse E, Ferguson N, Herron C E et al. Amyloid beta-protein dimers rapidly form stable synaptotoxic protofibrils. J Neurosci 2010; 30: 14411-14419.
46. Roberts R O, Geda Y E, Knopman D S, Cha R H, Pankratz V S, Boeve B F et al. The Mayo Clinic Study of Aging: design and sampling, participation, baseline measures and sample characteristics. Neuroepidemiology 2008; 30: 58-69.
47. Petersen R C, Roberts R O, Knopman D S, Geda Y E, Cha R H, Pankratz V S et al. Prevalence of mild cognitive impairment is higher in men: The Mayo Clinic Study of Aging. Neurology 2010; 75: 889-897.
48. St Sauver J L, Grossardt B R, Yawn B P, Melton III L J, Pankratz J J, Brue S M et al. Data resource profile: the Rochester Epidemiology Project (REP) medical records-linkage system. International journal of epidemiology 2012; 41: 1614-1624.
49. Brinkmalm G, Hong W, Wang Z, Liu W, O'Malley T T, Sun X et al. Identification of neurotoxic cross-linked amyloid-β dimers in the Alzheimer's brain. Brain 2019; 142: 1441-1457.
50. Liu L, Ding L, Rovere M, Wolfe M S, Selkoe D J. A cellular complex of BACE1 and γ-secretase sequentially generates Aβ from its full-length precursor. Journal of Cell Biology 2019; 218: 644-663.
51. Liu L, Lauro B M, Wolfe M S, Selkoe D J. Hydrophilic loop 1 of Presenilin-1 and the APP GxxxG transmembrane motif regulate γ-secretase function in generating Alzheimer-causing Aβ peptides. Journal of Biological Chemistry 2021; 100393.
52. Jin M, O'Nuallain B, Hong W, Boyd J, Lagomarsino V N, O'Malley T T et al. An in vitro paradigm to assess potential anti-Aβ antibodies for Alzheimer's disease. Nature communications 2018; 9: 1-13.
53. Yang T, Li S, Xu H, Walsh D M, Selkoe D J. Large soluble oligomers of amyloid β-protein from Alzheimer brain are far less neuroactive than the smaller oligomers to which they dissociate. Journal of Neuroscience 2017; 37: 152-163.
54. Li S, Jin M, Koeglsperger T, Shepardson N E, Shankar G M, Selkoe D J. Soluble Aβ oligomers inhibit long-term potentiation through a mechanism involving excessive activation of extrasynaptic NR2B-containing NMDA receptors. Journal of Neuroscience 2011; 31: 6627-6638.
55. Wang M J, Yi S, Han J-y, Park S Y, Jang J-W, Chun I K et al. Oligomeric forms of amyloid-β protein in plasma as a potential blood-based biomarker for Alzheimer's disease. Alzheimer's research & therapy 2017; 9: 1-10.
56. Meng X, Li T, Wang X, Lv X, Sun Z, Zhang J et al. Association between increased levels of amyloid-β oligomers in plasma and episodic memory loss in Alzheimer's disease. Alzheimer's research & therapy 2019; 11: 1-10.
57. Youn Y C, Lee B S, Kim G J, Ryu J S, Lim K, Lee R et al. Blood amyloid-β oligomerization as a biomarker of Alzheimer's disease: A blinded validation study. Journal of Alzheimer's Disease 2020; 1-7.
58. Aksenova, M. V., Aksenov, M. Y., Butterfield, D. A., & Carney, J. M. (1996) alpha-1-antichymotrypsin interaction with A beta (1-40) inhibits fibril formation but does not affect the peptide toxicity. Neurosci. Lett. 211, 45-48.
59. Anderson, C. C., Carroll, J. M., Gallucci, S., Ridge, J. P., Cheever, A. W., & Matzinger, P. (2001a) Testing time-, ignorance-, and danger-based models of tolerance. Journal of Immunology (Baltimore, Md.: 1950) 166, 3663-3671.
60. Anderson, C. C., & Matzinger, P. (2001b) Immunity or tolerance: opposite outcomes of microchimerism from skin grafts. Nature Medicine 7, 80-87.
61. Anderton, B. H., Callahan, L., Coleman, P., Davies, P., Flood, D., Jicha, G. A., Ohm, T., & Weaver, C. (1998) Dendritic changes in Alzheimer's disease and factors that may underlie these changes. Prog. Neurobiol. 55, 595-609.
62. Antzutkin, O. N., Balbach, J. J., Leapman, R. D., Rizzo, N. W., Reed, J., & Tycko, R. (2000) Multiple quantum solid-state NMR indicates a parallel, not antiparallel, organization of beta-sheets in Alzheimer's beta-amyloid fibrils. Proceedings of the National Academy of Sciences of the United States of America 97, 13045-13050.
63. Brusic, V., Rudy, G., & Harrison, L. C. (1998) MHCPEP, a database of MHC-binding peptides: update 1997. Nucleic Acids Research (Online) 26, 368-371.
64. Chapman, P. F., White, G. L., Jones, M. W., Cooper-Blacketer, D., Marshall, V. J., Irizarry, M., Younkin, L., Good, M. A., Bliss, T. V., Hyman, B. T., Younkin, S. G., & Hsiao, K. K. (1999) Impaired synaptic plasticity and learning in aged amyloid precursor protein transgenic mice. Nat Neurosci 2, 271-276.
65. Cummings, J. L., Vinters, H. V., Cole, G. M., & Khachaturian, Z. S. (1998) Alzheimer's disease: etiologies, pathophysiology, cognitive reserve, and treatment opportunities. Neurology 51, S2-17; discussion S65-17.
66. den_Boer, A. T., Diehl, L., van_Mierlo, G. J., van_der_Voort, E. I., Fransen, M. F., Krimpenfort, P., Melief, C. J., Offringa, R., & Toes, R. E. (2001) Longevity of antigen presentation and activation status of APC are decisive factors in the balance between CTL immunity versus tolerance. Journal of Immunology (Baltimore, Md.: 1950) 167, 2522-2528.
67. Esiri, M., Hyman, B., Beyreuther, K., & Masters, C. (1997) Aging and dementia. In: Greenfield's Neuropathology (Graham D, Lantos P, eds), pp 153-233. London: Arnold.
68. Giulian, D., Haverkamp, L. J., Yu, J. H., Karshin, W., Tom, D., Li, J., Kirkpatrick, J., Kuo, L. M., & Roher, A. E. (1996) Specific domains of beta-amyloid from Alzheimer plaque elicit neuron killing in human microglia. J. Neurosci. 16, 6021-6037.
69. Gomez-Isla, T., Price, J. L., McKeel, D. W., Morris, J. C., Growdon, J. H., & Hyman, B. T. (1996) Profound loss of layer II entorhinal cortex neurons occurs in very mild Alzheimer's disease. The Journal of Neuroscience: the Official Journal of the Society For Neuroscience 16, 4491-4500.
70. Green, D. R. (1998) Apoptotic pathways: the roads to ruin. Cell 94, 695-698.
71. Haass, C., Schlossmacher, M., Hung, A. Y., Vigo-Pelfrey, C., Mellon, A., Ostaszewski, B., Lieberburg, I., Koo, E. H., Schenk, D., Teplow, D., & Selkoe, D. (1992) Amyloid b-peptide is produced by cultured cells during normal metabolism. Nature 359, 322-325.
72. Harper, J. D., Lieber, C. M., & Lansbury P T, J. r. (1997a) Atomic force microscopic imaging of seeded fibril formation and fibril branching by the Alzheimer's disease amyloid-beta protein. Chem. Biol. 4, 951-959.
73. Harper, J. D., Wong, S. S., Lieber, C. M., & Lansbury Jr, P. T. (1997b) Observation of metastable Ab amyloid protofibrils by atomic force microscopy. Chemistry & Biology 4, 119-125.
74. Hartley, D., Walsh, D. M., Ye, C. P., Diehl, T., Vasquez, S., Vassilev, P. M., Teplow, D. B., & Selkoe, D. J. (1999) Protofibrillar intermediates of amyloid b-protein induce acute electrophysiological changes and progressive neurotoxicity in cortical neurons. J. Neurosci. 19, 8876-8884.
75. Hartley, D. M., Kurth, M. C., Bjerkness, L., Weiss, J. H., & Choi, D. W. (1993) Glutamate receptor-induced ⁴⁵Ca²⁺ accumulation in cortical cell culture correlates with subsequent neuronal degeneration. J. Neurosci. 13, 1993-2000.
76. Holcomb, L., Gordon, M. N., McGowan, E., Yu, X., Benkovic, S., Jantzen, P., Wright, K., Saad, I., Mueller, R., Morgan, D., Sanders, S., Zehr, C., O'Campo, K., Hardy, J., Prada, C. M., Eckman, C., Younkin, S., Hsiao, K., & Duff, K. (1998) Accelerated Alzheimer-type phenotype in transgenic mice carrying both mutant amyloid precursor protein and presenilin 1 transgenes. Nat. Med. 4, 97-100.
77. Honeyman, M. C., Brusic, V., Stone, N. L., & Harrison, L. C. (1998) Neural network-based prediction of candidate T-cell epitopes. Nature Biotechnology 16, 966-969.
78. Horvath, A., Olive, C., Wong, A., Clair, T., Yarwood, P., Good, M., & Toth, I. (2002) Lipoamino acid-based adjuvant carrier system: enhanced immunogenicity of group a streptococcal peptide epitopes. Journal of Medicinal Chemistry 45, 1387-1390.
79. Hsia, A. Y., Masliah, E., McConlogue, L., Yu, G. Q., Tatsuno, G., Hu, K., Kholodenko, D., Malenka, R. C., Nicoll, R. A., & Mucke, L. (1999) Plaque-independent disruption of neural circuits in Alzheimer's disease mouse models. Proc Natl Acad Sci USA 96, 3228-3233.
80. Huang, W., Nardelli, B., & Tam, J. P. (1994) Lipophilic multiple antigen peptide system for peptide immunogen and synthetic vaccine. Molecular Immunology 31, 1191-1199.
81. Huppertz, B., Frank, H. G., & Kaufmann, P. (1999) The apoptosis cascade-morphological and immunohistochemical methods for its visualization. Anatomy And Embryology 200, 1-18.
82. Ivins, K. J., Bui, E. T., & Cotman, C. W. (1998) Beta-amyloid induces local neurite degeneration in cultured hippocampal neurons: evidence for neuritic apoptosis. Neurobiology Of Disease 5, 365-378.
83. Kim, S. H., Wang, R., Gordon, D. J., Bass, J., Steiner, D. F., Lynn, D. G., Thinakaran, G., Meredith, S. C., & Sisodia, S. S. (1999) Furin mediates enhanced production of fribrillogenic ABri peptides in familial British dementia. Nature Neurosci. 2, 984-988.
84. Klinman, D. M. (1998) Therapeutic applications of CpG-containing oligodeoxynucleotides. Antisense & Nucleic Acid Drug Development 8, 181-184.
85. Krieg, A. M., Love_Homan, L., Yi, A. K., & Harty, J. T. (1998) CpG DNA induces sustained IL-12 expression in vivo and resistance to Listeria monocytogenes challenge. Journal of Immunology (Baltimore, Md.: 1950) 161, 2428-2434.
86. Lambert, M. P., Barlow, A. K., Chromy, B. A., Edwards, C., Freed, R., Liosatos, M., Morgan, T. E., Rozovsky, I., Trommer, B., Viola, K. L., Wals, P., Zhang, C., Finch, C. E., Kraff, G. A., & Klein, W. L. (1998) Diffusible, nonfribrillar ligands derived from Ab₁₋₄₂ are potent central nervous system neurotoxins. Proc. Natl. Acad. Sci. USA 95, 6448-6453.
87. Lansbury, P. T., Costa, P. R., Griffiths, J. M., Simon, E. J., Auger, M., Halverson, K. J., Kocisko, D. A., Hendsch, Z. S., Ashburn, T. T., & Spencer, R. G. (1995) Structural model for the beta-amyloid fibril based on interstrand alignment of an antiparallel-sheet comprising a C-terminal peptide. Nature Structural Biology 2, 990-998.
88. Lansbury, P. T., & Kosik, K. S. (2000) Neurodegeneration: new clues on inclusions. Chemistry and Biology 7, R9-R12.
89. Larsson, A., Balow, R. M., Lindahl, T. L., & Forsberg, P. O. (1993) Chicken antibodies: taking advantage of evolution—a review. Poultry Science 72, 1807-1812.
90. Lillehoj, H. S., Sasai, K., & Matsuda, H. (1994) Development and characterization of chicken-chicken B cell hybridomas secreting monoclonal antibodies that detect sporozoite and merozoite antigens of Eimeria. Poultry Science 73, 1685-1693.
91. Lorenzo, A., & Yankner, B. (1994) b-amyloid neurotoxicity requires fibril formation and is inhibited by Congo red. Proc. Natl. Acad. Sci. USA 91, 12243-12247.
92. Lue, L. F., Kuo, Y. M., Roher, A. E., Brachova, L., Shen, Y., Sue, L., Beach, T., Kurth, J. H., Rydel, R. E., & Rogers, J. (1999) Soluble amyloid beta peptide concentration as a predictor of synaptic change in Alzheimer's disease. Amer. J. Path. 155, 853-862.
93. MacGregor, I. (2001) Prion protein and developments in its detection. Transfusion Medicine (Oxford, England) 11, 3-14.
94. Malenka, R. C., & Nicoll, R. A. (1999) Long-term potentiation—a decade of progress? Science 285, 1870-1874.
95. Matsushita, K., Horiuchi, H., Furusawa, S., Horiuchi, M., Shinagawa, M., & Matsuda, H. (1998) Chicken monoclonal antibodies against synthetic bovine prion protein peptide. The Journal of Veterinary Medical Science/the Japanese Society of Veterinary Science 60, 777-779.
96. Mattson, M. P., Barger, S. W., Cheng, B., Lieberburg, I., Smith-Swintosky, V. L., & Rydel, R. E. (1993a) Beta-amyloid precursor protein metabolites and loss of neuronal Ca²⁺ homeostasis in Alzheimer's disease. Trends in Neuroscience 16, 409-414.
97. Mattson, M. P., Keller, J. N., & Begley, J. G. (1998) Evidence for synaptic apoptosis. Experiment. Neurol. 153, 35-48.
98. Mattson, M. P., LaFerla, F. M., Chan, S. L., Leissring, M. A., Shepel, P. N., & Geiger, J. D. (2000) Calcium signaling in the E R: its role in neuronal plasticity and neurodegenerative disorders. Trends in Neurosciences 23, 222-229.
99. Mattson, M. P., Tomaselli, K. J., & Rydel, R. E. (1993b) Calcium-destabilizing and neurodegenerative effects of aggregated beta-amyloid peptide are attenuated by basic FGF. Brain Res. 621, 35-49.
100. Matzinger, P. Essay 1: the Danger model in its historical context. Scandinavian Journal of Immunology 54, 4-9.
101. Matzinger, P. Introduction to the series. Danger model of immunity. Scandinavian Journal of Immunology 54, 2-3.
102. Oda, T., Wals, P., Osterburg, H. H., Johnson, S. A., Pasinetti, G. M., Morgan, T. E., Rozovsky, I., Stine, W. B., Snyder, S. W., Holzman, T. F., & et, a. (1995) Clusterin (apoJ) alters the aggregation of amyloid beta-peptide (A beta 1-42) and forms slowly sedimenting A beta complexes that cause oxidative stress. Exp. Neurol. 136, 22-31.
103. Olive, C., Batzloff, M. R., Horvath, A., Wong, A., Clair, T., Yarwood, P., Toth, I., & Good, M. F. (2002) A lipid core peptide construct containing a conserved region determinant of the group A streptococcal M protein elicits heterologous opsonic antibodies. Infection and Immunity 70, 2734-2738.
104. Pike, C. J., Burdick, D., Walencewicz, A. J., Glabe, C. G., & Cotman, C. W. (1993) Neurodegeneration induced by ß-amyloid peptides in vitro: the role of peptide assembly state. J. Neurosci. 13, 1676-1687.
105. Pitschke, M., Prior, R., Haupt, M., & Riesner, D. (1998) Detection of single amyloid B-protein aggregates in the cerebrospinal fluid of Alzheimer's patients by fluorescence correlation spectroscopy. Nat. Med. 4, 832-834.
106. Podlisny, M. B., Ostaszewski, B., Haass, C. H., & Selkoe, D. J. (1995) Effects of Congo red on Ab production, stability and aggregation in CHO cells. Soc Neurosci. 21, 1281.
107. Podlisny, M. B., Walsh, D. M., Amarante, P., Ostaszewski, B. L., Stimson, E. R., Maggio, J. E., Teplow, D. B., & Selkoe, D. J. (1998) Oligomerization of endogenous and synthetic amyloid b-protein at nanomolar levels in cell culture and stabilization of monomer by Congo red. Biochem. 37, 3602-3611.
108. Rankin, R., Pontarollo, R., Ioannou, X., Krieg, A. M., Hecker, R., Babiuk, L. A., & van_Drunen_Littel_van_den_Hurk, S. (2001) CpG motif identification for veterinary and laboratory species demonstrates that sequence recognition is highly conserved. Antisense & Nucleic Acid Drug Development 11, 333-340.
109. Riek, R., Hornemann, S., Wider, G., Billeter, M., Glockshuber, R., & Wuthrich, K. (1996) NMR structure of the mouse prion protein domain PrP(121-321). Nature 382, 180-182.
110. Roher, A. E., Chaney, M. O., Kuo, Y.-M., Webster, S. D., Stine, W. B., Haverkamp, L. J., Woods, A. S., Cotter, R. J., Tuohy, J. M., Krafft, G. A., Bonnell, B. S., & Emmerling, M. R. (1996) Morphology and toxicity of Ab-(1-42) dimer derived from neuritic and vascular amyloid deposits of Alzheimer's disease. J. Biol. Chem. 271, 20631-20635.
111. Saragovi, H. U., Greene, M. I., Chrusciel, R. A., & Kahn, M. (1992) Loops and secondary structure mimetics: development and applications in basic science and rational drug design. 10, 773-778.
112. Shoji, M., Golde, T. E., Ghiso, J., Cheung, T. T., Estus, S., Shaffer, L. M., Cai, X., McKay, D. M., Tintner, R., Frangione, B., & Younkin, S. G. (1992) Production of the Alzheimer amyloid b protein by normal proteolytic processing. Science 258, 126-129.
113. Sze, C. I., Troncoso, J. C., Kawas, C., Mouton, P., Price, D. L., & Martin, L. J. (1997) Loss of the presynaptic vesicle protein synaptophysin in hippocampus correlates with cognitive decline in Alzheimer disease. J. Neuropath. Exp. Neurol. 56, 933-944.
114. Takeshita, S., Takeshita, F., Haddad, D. E., Ishii, K. J., & Klinman, D. M. (2000) CpG oligodeoxynucleotides induce murine macrophages to up-regulate chemokine mRNA expression. Cellular Immunology 206, 101-106.
115. Teplow, D. B. (1998) Structural and kinetic features of amyloid beta-protein fibrillogenesis. Amyloid 5, 121-142.
116. Terry, R. D. (1963) The fine structure of neurofibrillary tangles in Alzheimer's disease. J. Neuropathol. Exp. Neurol 22, 629-642.
117. Van Engeland, M., Nieland, L. J., Ramaekers, F. C., Schutte, B., & Reutelingsperger, C. P. (1998) Annexin V-affinity assay: a review on an apoptosis detection system based on phosphatidylserine exposure. Cytometry 31, 1-9.
118. Walsh, D. M., Hartley, D. M., Kusumoto, Y., Fezoui, Y., Condron, M. M., Lomakin, A., Benedek, G. B., Selkoe, D. J., & Teplow, D. B. (1999) Amyloid beta-protein fibrillogenesis. Structure and biological activity of protofibrillar intermediates. J. Biol. Chem. 274, 25945-25952.
119. Walsh, D. M., Lomakin, A., Benedek, G. B., Maggio, J. E., Condron, M. M., & Teplow, D. B. (1997) Amyloid b-protein fibrillogenesis: Detection of a protofibrillar intermediate. J. Biol. Chem. 272, 22364-22374.
120. Walsh, D. M., Tseng, B. P., Rydel, R. E., Podlisny, M. B., & Selkoe, D. J. (2000) Detection of intracellular oligomers of amyloid ß-protein in cells derived from human brain. Biochemistry 39, 10831-10839.
121. Wernette, C. M., Smith, B. F., Barksdale, Z. L., Hecker, R., & Baker, H. J. (2002) CpG oligodeoxynucleotides stimulate canine and feline immune cell proliferation. Veterinary Immunology and Immunopathology 84, 223-236.
122. Xia, W., Zhang, J., Kholodenko, D., Citron, M, Podlisny, M. B., Teplow, D. B., Haass, C., Seubert, P., Koo, E. H., & Selkoe, D. J. (1997) Enhanced production and oligomerization of the 42-residue amyloid b-protein by Chinese hamster ovary cells stably expressing mutant presenilins. J. Biol. Chem. 272, 7977-7982.
123. Yamamoto, S., Yamamoto, T., Kataoka, T., Kuramoto, E., Yano, O., & Tokunaga, T. (1992) Unique palindromic sequences in synthetic oligonucleotides are required to induce IFN [correction of INF] and augment IFN-mediated [correction of INF] natural killer activity. Journal of Immunology (Baltimore, Md.: 1950) 148, 4072-4076.
124. Yang, F., Sun, X., Beech, W., Teter, B., Wu, S., Sigel, J., Vinters, H. V., Frautschy, S. A., & Cole, G. M. (1998) Antibody to caspase-cleaved actin detects apoptosis in differentiated neuroblastoma and plaque-associated neurons and microglia in Alzheimer's disease [see comments]. American Journal Of Pathology 152, 379-389.
125. Ye, C., Walsh, D., Selkoe, D., & Hartley, D. (2000) Protofibrillar amyloid ß-protein induce acute electrophysiological changes in cortical neurons: NMDA vs. non-NMDA activation. J Neurosci. 1999 Oct. 15; 19(20): 8876-8884.
126. Zinkernagel, R. M., & Hengartner, H. (2001) Regulation of the immune response by antigen. Science 293, 251-253.
127. “2021 Alzheimer's disease facts and figures.” Alzheimers Dement. 2021 March; 17(3):327-406.
128. Cummings, J., Aisen, P. S., DuBois, B. et al. Drug development in Alzheimer's disease: the path to 2025. Alz Res Therapy 8, 39 (2016).
129. Tónnies, E, & Trushina, E. ‘Oxidative Stress, Synaptic Dysfunction, and Alzheimer's Disease’. J Alzheimers Dis. 2017; 57(4):1105-1121.
130. Tu, S., Okamoto, S i., Lipton, S. A. et al. Oligomeric Aβ-induced synaptic dysfunction in Alzheimer's disease. Mol Neurodegeneration 9, 48 (2014).
131. Braak H, Braak E. Evolution of the neuropathology of Alzheimer's disease. Acta Neurol Scand Suppl. 1996; 165:3-12.
132. Perl D P. Neuropathology of Alzheimer's disease. Mt Sinai J Med. 2010 January-February; 77(1):32-42.
133. Serrano-Pozo, A., Frosch, M. P., Masliah, E., & Hyman, B. T. (2011). Neuropathological alterations in Alzheimer disease. Cold Spring Harbor perspectives in medicine, 1(1), a006189. doi.org/10.1101/cshperspect.a006189
134. Zheng W H, Bastianetto S, Mennicken F, Ma W, Kar S. Amyloid beta peptide induces tau phosphorylation and loss of cholinergic neurons in rat primary septal cultures. Neuroscience. 2002; 115(1):201-11.
135. Wu H Y, Kuo P C, Wang Y T, Lin H T, Roe A D, Wang B Y, Han C L, Hyman B T, Chen Y J, Tai H C. β-Amyloid Induces Pathology-Related Patterns of Tau Hyperphosphorylation at Synaptic Terminals. J Neuropathol Exp Neurol. 2018 Sep. 1; 77(9):814-826.
136. Viola, K. L., & Klein, W. L. (2015). Amyloid β oligomers in Alzheimer's disease pathogenesis, treatment, and diagnosis. Acta neuropathologica, 129(2), 183-206.
137. Bloom G S. Amyloid-β and tau: the trigger and bullet in Alzheimer disease pathogenesis. JAMA Neurol. 2014 April; 71(4):505-8.
138. Hong, W., Wang, Z., Liu, W., O'Malley, T. T., Jin, M., Willem, M., Haass, C., Frosch, M. P., & Walsh, D. M. (2018). Diffusible, highly bioactive oligomers represent a critical minority of soluble Aβ in Alzheimer's disease brain. Acta neuropathologica, 136(1), 19-40.
139. Cline E N, Bicca M A, Viola K L, Klein W L. The Amyloid-β Oligomer Hypothesis: Beginning of the Third Decade. J Alzheimers Dis. 2018; 64(s1):5567-5610.
140. Cleary J P, Walsh D M, Hofmeister J J, Shankar G M, Kuskowski M A, Selkoe D J, Ashe K H. Natural oligomers of the amyloid-beta protein specifically disrupt cognitive function. Nat Neurosci. 2005 January; 8(1):79-84.
141. Shankar G M, Li S, Mehta T H, Garcia-Munoz A, Shepardson N E, Smith I, Brett F M, Farrell M A, Rowan M J, Lemere C A, Regan C M, Walsh D M, Sabatini B L, Selkoe D J. Amyloid-beta protein dimers isolated directly from Alzheimer's brains impair synaptic plasticity and memory. Nat Med. 2008 August; 14(8):837-42.
142. O'Malley, T. T., Witbold, W. M., 3rd, Linse, S., & Walsh, D. M. (2016). The Aggregation Paths and Products of Aβ42 Dimers Are Distinct from Those of the Aβ42 Monomer. Biochemistry, 55(44), 6150-6161.
143. Li S, Jin M, Liu L, Dang Y, Ostaszewski B L, Selkoe D J. Decoding the synaptic dysfunction of bioactive human A D brain soluble Aβ to inspire novel therapeutic avenues for Alzheimer's disease. Acta Neuropathol Commun. 2018 Nov. 8; 6(1):121.
144. Morgan D. Immunotherapy for Alzheimer's disease. J Intern Med. 2011 January; 269(1):54-63.
145. Arndt, J. W., Qian, F., Smith, B. A. et al. Structural and kinetic basis for the selectivity of aducanumab for aggregated forms of amyloid-β. Sci Rep 8, 6412 (2018).
146. Sevigny, J., Chiao, P., Bussière, T. et al. The antibody aducanumab reduces Aβ plaques in Alzheimer's disease. Nature 537, 50-56 (2016).
147. Knopman D S, Jones D T, Greicius M D. Failure to demonstrate efficacy of aducanumab: An analysis of the EMERGE and ENGAGE trials as reported by Biogen, December 2019. Alzheimers Dement. 2021 April; 17(4):696-701.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A synthetic peptide immunogen for stimulating development of antibodies, wherein the synthetic peptide immunogen is specific to conformational epitopes present in an aggregated form of the amyloid β protein (Aβ) comprising oligomers of amyloid beta protein (oAβ), but not the monomeric form.

2. The synthetic peptide immunogen of claim 1, comprising the isolated amyloid beta sequence GYEVHHQKLV (SEQ ID NO:1), or a sequence YEXHH (SEQ ID NO:41), wherein X is a hydrophobic amino acid (V, I, or L).

3. The synthetic peptide immunogen of claim 1, comprising the peptide sequence CGKCGYEVHHQKLVPNVLKQHHVEYGCK (SEQ ID NO:2) or CGKCGYEVHHQKLVPNGYEVHHQKLVCK (SEQ ID NO:3), or a peptide sequence of one of SEQ ID NOs:5-17, as shown in Table A.

4. The synthetic peptide of claim 3, wherein the peptide further comprises a carrier protein linked to N terminus, optionally wherein the carrier protein is linked by maleimide.

5. The synthetic peptide of claim 4, wherein the carrier protein comprises Keyhole Limpet Hemocyanin (KLH), bovine serum albumin (BSA), or ovalbumin (OVA).

6. The synthetic peptide of claim 4, wherein the carrier protein comprises a peptide having the sequence KSSKSKKKFISEAIIHHLHSRHPGK (SEQ ID NO::4), optionally with (i) a Pam3Cys (N-α-Palmitoyl-S-2,3-bis(palmitoyloxy)-(2RS)-propyl-L-cysteine, Palmitoyl-Cys((RS)-2,3) attached to the second lysine (K4), wherein the carrier protein is conjugated to the N terminus via maleimide.

7. A synthetic peptide having the sequence KSSKSKKKFISEAIIHHLHSRHPGK (SEQ ID NO:4), optionally with (i) a Pam3Cys (N-α-Palmitoyl-S-2,3-bis(palmitoyloxy)-(2RS)-propyl-L-cysteine, Palmitoyl-Cys((RS)-2,3) attached to the second lysine (K4).

8. A method for detecting oAβ in a sample comprising one or more biological fluids, preferably a sample from a human subject, the method comprising:
contacting the sample with capture antibodies bound to a surface, wherein the capture antibodies comprise 71A1 or 1G5 antibodies;
contacting the sample with a labeled detection antibody comprising a detectable label, wherein the detection antibody binds to the capture antibody-oAβ complexes, under conditions sufficient for the detection antibody to bind to the capture antibody-oAβ complexes; and
detecting and optionally quantifying the detectable label, wherein the amount of detectable label is proportional to the amount of oAβ in the sample, thereby detecting oAβ in the sample.

9. The method of claim 8, wherein the one or more biological fluids comprise blood or plasma.

10. The method of claim 8, wherein the surface is the surface of a plate or a bead.

11. The method of claim 10, wherein the plate is a multiwell plate or a test strip.

12. The method of claim 10, wherein the bead is selected from magnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, or TEFLON® beads.

13. The method of claim 8, wherein the capture antibody is conjugated to a biotin moiety, the surface is coated with streptavidin, and the antibody is attached to the surface via biotin-streptavidin binding.

14. The method of claim 8, wherein the detection antibody binds to Aβ at a different epitope from the capture antibody.

15. The method of claim 8, wherein the capture antibody comprises an affinity tag, and the detection antibody binds to the affinity tag.

16. The method of claim 8, wherein the detectable label is a radioisotope, a chemiluminescent substance, a fluorescent substance, a metal complex, a bioluminescent substance such as a luciferase, or a nucleic acid.

17. The method of any of claim 8, wherein detecting and optionally quantifying the concentration of oAβ in the sample comprises using a radio immunoassay method (RIA) when the detection antibody is labeled with a radioisotope, a chemiluminescent immunoassay method (CIA) when the detection antibody is labeled with a chemiluminescent substance, a fluorescent immunoassay method (FIA) when the detection antibody is labeled with a fluorescent substance, an electro-chemiluminescent immunoassay method (ECLIA) when the detection antibody is labeled with a metal complex, a bioluminescent immunoassay method (BLIA) when the detection antibody is labeled with a bioluminescent substance such as a luciferase, an immuno-PCR comprising amplifying a nucleic acid that labeled an antibody by PCR and detecting the nucleic acid, when the detection antibody is labeled with a nucleic acid, a turbidimetric immunoassay method (TAI) detecting turbidity occurred by forming an immunocomplex, a latex agglutination turbidimetric assay (LA) comprising detecting latex aggregated by formation of complexes, or an immunochromatography assay utilizing a reaction on a cellulose membrane.

18. A kit comprising:
a surface, optionally a plate or beads;
a capture antibody selected from 71A1 or 1G5, optionally linked to the surface;
a detection antibody; and
a standard comprising a known concentration of oAβ.

19. The kit of claim 18, wherein the surface is the surface of a plate or a bead.

20. The kit of claim 19, wherein the plate is a multiwell plate or a test strip.

21. The kit of claim 19, wherein the bead is selected from magnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, or TEFLON® beads.

22. The kit of claim 18, wherein the capture antibody is conjugated to a biotin moiety, the surface is coated with streptavidin, and the antibody is attached to the surface via biotin-streptavidin binding.

23. The kit of claim 18, wherein the detection antibody binds to Aβ at a different epitope from the capture antibody.

24. The kit of claim 18, wherein the capture antibody comprises an affinity tag, and the detection antibody binds to the affinity tag.

25. The kit of claim 18, wherein the detectable label is a radioisotope, a chemiluminescent substance, a fluorescent substance, a metal complex, a bioluminescent substance such as a luciferase, or a nucleic acid.

26. A method for treating a neurological disorder associated with accumulation of oAβ in a subject, the method comprising administering a therapeutically effective amount of 71A1 antibody, or an antigen-binding fragment thereof, or 1G5 antibody, or an antigen-binding fragment thereof to the subject.

27. The method of claim 26, wherein the disorder is cerebral amyloid angiopathy (CAA) and Alzheimer's disease (AD).

28. The method of claim 26, wherein the antibody is administered to the brain of the subject.

29. The method of claim 26, wherein the antibody is a monoclonal antibody, a humanized antibody, or a chimeric antibody.
